SEQUENCE LISTING (24 sequences)

SEQ ID NO: 1 - protein, 6 aa, Artificial Sequence
  Synthetic Polypeptide
  MISC_FEATURE: LC CDR1
QDVNTA

SEQ ID NO: 2 - protein, 3 aa, Artificial Sequence
  Synthetic Polypeptide
  MISC_FEATURE: LC CDR2
SAS

SEQ ID NO: 3 - protein, 9 aa, Artificial Sequence
  Synthetic Polypeptide
  MISC_FEATURE: LC CDR3
QQHYTTPPT

SEQ ID NO: 4 - protein, 8 aa, Artificial Sequence
  Synthetic Polypeptide
  MISC_FEATURE: HC CDR1
GFNIKDTY

SEQ ID NO: 5 - protein, 8 aa, Artificial Sequence
  Synthetic Polypeptide
  MISC_FEATURE: HC CDR2
IYPTNGYT

SEQ ID NO: 6 - protein, 13 aa, Artificial Sequence
  Synthetic Polypeptide
  MISC_FEATURE: HC CDR3
SRWGGDGFYAMDY

SEQ ID NO: 7 - protein, 107 aa, Artificial Sequence [sequence omitted]

SEQ ID NO: 8 - protein, 120 aa, Artificial Sequence [sequence omitted]

SEQ ID NO: 9-10 - skipped (2 entries)

SEQ ID NO: 11 - protein, 10 aa, Artificial Sequence
  Synthetic Polypeptide
  MISC_FEATURE: LC CDR1
RASSSVSYIH

SEQ ID NO: 12 - protein, 7 aa, Artificial Sequence
  Synthetic Polypeptide
  MISC_FEATURE: LC CDR2
ATSNLAS

SEQ ID NO: 13 - protein, 9 aa, Artificial Sequence
  Synthetic Polypeptide
  MISC_FEATURE: LC CDR3
QQWTSNPPT

SEQ ID NO: 14 - protein, 5 aa, Artificial Sequence
  Synthetic Polypeptide
  MISC_FEATURE: HC CDR1
SYNMH

SEQ ID NO: 15 - protein, 17 aa, Artificial Sequence
  Synthetic Polypeptide
  MISC_FEATURE: HC CDR2
AIYPGNGDTSYNQKFKG

SEQ ID NO: 16 - protein, 12 aa, Artificial Sequence
  Synthetic Polypeptide
  MISC_FEATURE: HC CDR3
STYYGGDWYFNV

SEQ ID NO: 17 - protein, 106 aa, Artificial Sequence [sequence omitted]

SEQ ID NO: 18 - protein, 121 aa, Artificial Sequence [sequence omitted]

SEQ ID NO: 19-20 - skipped (2 entries)

SEQ ID NO: 21 - protein, 214 aa, Artificial Sequence
  Synthetic Polypeptide
  MISC_FEATURE: VL-IgGKappa
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 450 aa, Artificial Sequence
  Synthetic Polypeptide
  MISC_FEATURE: VH-IgG1
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 213 aa, Artificial Sequence
  Synthetic Polypeptide
  MISC_FEATURE: VL-IgGKappa
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 451 aa, Artificial Sequence
  Synthetic Polypeptide
  MISC_FEATURE: VH-IgG1
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2021/0155710 A1
(43) Pub. Date: May 27, 2021

(54) MODULATING ANTIBODY DEPENDENT CELLULAR PHAGOCYTOSIS

(71) Applicant: Amgen Inc., Thousand Oaks, CA (US)

(72) Inventors: Scott KUHNS, Newbury Park, CA (US); Junyan SHU, Newbury Park, CA (US); Qingchun ZHANG, Oak Park, CA (US)

(21) Appl. No.: 16/972,298

(22) PCT Filed: Jun. 5, 2019
(86) PCT No.: PCT/US2019/035643

§ 371 Date: Dec. 4, 2020

Related U.S. Application Data
(60) Provisional application No. 62/680,934, filed on Jun. 5, 2018.

(51) Int. Cl. C07K 16/32; C07K 16/28

(52) U.S. Cl. CPC C07K 16/32; C07K 16/2887; C07K 2317/41; C07K 2317/72; C07K 2317/24; C07K 2317/71; C07K 2317/73

(57) ABSTRACT

Provided herein are methods of modulating Antibody Dependent Cellular Phagocytosis (ADCP) activity of an antibody composition. In exemplary embodiments, the method comprises modulating the amount of (a) galactosylated glycans of the antibody, (b) afucosylated glycans of the antibody, (c) high mannose glycans of the antibody, or (d) a combination thereof, to modulate ADCP activity of the antibody, as further described herein.

### FIELD OF THE INVENTION

The present invention relates generally to modulating Antibody Dependent Cellular Phagocytosis (ADCP) effector function of IgG1 antibodies, including trastuzumab and rituximab.

### INCORPORATION BY REFERENCE OF MATERIAL SUBMITTED ELECTRONICALLY

Incorporated by reference in its entirety is a computer-readable nucleotide/amino acid sequence listing submitted concurrently herewith in computer readable format (CRF) and identified as follows: 20 kilobytes ASCII (Text) file named A-2236-US-PSP_SeqlistingFinal.txt; created on Jun. 5, 2018.

### BACKGROUND

Throughout the development and manufacturing process of therapeutic monoclonal antibodies, it is essential to appropriately design and monitor the key critical quality attributes that will result in the desired safety and efficacy of the product. A critical quality attribute (CQA) has been defined as “a physical, chemical, biological, or microbiological property or characteristic that should be within an appropriate limit, range, or distribution to ensure the desired product quality”. Many important therapeutic monoclonal antibodies are of the IgG1 subclass and consequently poses the potential for Fc mediated effector function activities such as antibody-dependent cell-mediated cytotoxicity (ADCC), antibody-dependent cellular phagocytosis (ADCP) or complement-dependent cytotoxicity (CDC). See, e.g., Jiang, et al. Advances in the assessment and control of the effector functions of therapeutic antibodies. Nature reviews Drug discovery 2011; 10:101-11. Over the past approximately 15 years there has been a great deal of research into the quality attributes that impact effector function potential. It has become well established that the specific glycan structures associated with the conserved bi-antennary glycan in the Fc-CH2 domain can strongly influence the interaction with the FcγRs that mediate ADCC and ADCP and with C1q binding, the initial binding event leading to CDC (see Reusch D, Tejada M L. Fc glycans of therapeutic antibodies as critical quality attributes. Glycobiology 2015; 25:1325-34). For example, it has been demonstrated through structural, functional and binding studies that core fucose has a very significant impact on FcγRIIIa binding affinity, leading to substantial changes in ADCC activity (see Okazaki A, et al. Fucose depletion from human IgG1 oligosaccharide enhances binding enthalpy and association rate between IgG1 and FcgammaRIIIa. Journal of molecular biology 2004; 336:1239-49; Ferrara C, et al. Unique carbohydrate-carbohydrate interactions are required for high affinity binding between FcgammaRIII and antibodies lacking core fucose. Proceedings of the National Academy of Sciences of the United States of America 2011; 108:12669-74). More recently it has also been shown that high mannose and β-galactose levels can also play a role in modulating ADCC activity, though to a much more modest and less predictable extent than core fucose (Thomann M, et al. Fc-galactosylation modulates antibody-dependent cellular cytotoxicity of therapeutic antibodies. Molecular immunology 2016; 73:69-75). Similarly, CDC has been shown to be influenced by galactose via an impact to C1q binding (Hodoniczky J, et al. Control of recombinant monoclonal antibody effector functions by Fc N-glycan remodeling in vitro. Biotechnology progress 2005; 21:1644-52). Many of these observations have been enabled by a glycoengineering process wherein an engineered host cell line that makes afucosylated or a bisecting bi-antennary glycan structure on recombinant monoclonal antibodies are employed to generate test materials (Shinkawa T, et al. The absence of fucose but not the presence of galactose or bisecting N-acetylglucosamine of human IgG1 complex-type oligosaccharides shows the critical role of enhancing antibody-dependent cellular cytotoxicity. The Journal of biological chemistry 2003; 278:3466-73; Umana P, et al. Engineered glycoforms of an antineuroblastoma IgG1 with optimized antibody-dependent cellular cytotoxic activity. Nature biotechnology 1999; 17:176-80). More recently the use of glycan modification enzymes and affinity enrichment chromatography columns have been employed to create a sharply focused distribution of specific sub-structures that can be characterized in effector function assays. This approach has been deployed in the study of ADCC to good effect (see, e.g., Thomann M, et al. Molecular immunology 2016; 73:69-75). The monitoring and control of glycan species that affect effector function activity is also central to the development of biosimilars and follow-on biologics, representing key attributes and associated activities around which tight similarity criteria must be applied. A recent example where an originator product showed substantial drift in effector function related attributes has had an impact on efforts to develop a biosimilar candidate (see Kim S, et al. Drifts in ADCC-related quality attributes of Herceptin(R): Impact on development of a trastuzumab biosimilar. mAbs 2017; 9:704-14).

The contribution of ADCP in therapeutic antibody efficacy is not well understood, nor are the quality attributes that influence ADCP activity. The critical quality attributes that are the most impactful and predictive of ADCP activity, and therefore most suitable to monitor during IgG1 antibody manufacturing, are not well established.

### SUMMARY

The present disclosure is directed at elucidating the impact of various glycans (including, e.g., β-galactose, core-fucose and/or high mannose) on ADCP activity of IgG1 antibodies, including trastuzumab or rituximab. In some embodiments, the present disclosure provides methods of modulating (i.e. increasing or decreasing) ADCP activity of IgG1 antibodies (including methods of increasing or decreasing ADCP activity of trastuzumab or rituximab) by increasing or decreasing glycan species (including, e.g., enriching, increasing, removing and/or remodeling β-galactose, core-fucose and/or high mannose species). The present disclosure also provides anti-IgG1 antibodies having increased or decreased ADCP activity, and methods of matching the ADCP activity of a second IgG1 antibody to the ADCP activity of a reference antibody by modulating (i.e. increasing or decreasing) ADCP activity of the second IgG1 antibody (including trastuzumab or rituximab) by increasing or decreasing glycan species (including, e.g., enriching, increasing, removing and/or remodeling β-galactose, core-fucose and/or high mannose species). Cell culture methods useful in modulating (i.e. increasing or decreasing) ADCP activity of IgG1 antibodies (including increasing or decreasing ADCP activity of trastuzumab or rituximab) by increasing or decreasing glycan species (including, e.g., enriching, increasing, removing and/or remodeling β-galactose, core-fucose and/or high mannose species) are also disclosed.

In one aspect, the disclosure provides a method of modulating Antibody Dependent Cellular Phagocytosis (ADCP) activity of trastuzumab comprising increasing or decreasing the amount of terminal β-galactose in the antibody; or increasing or decreasing the amount of G1, G1a, G1b and/or G2 galactosylated species of the antibody.

In another aspect, the disclosure provides a method of increasing ADCP activity of trastuzumab comprising increasing the amount of terminal β-galactose in the antibody; or increasing the amount of G1, G1a, G1b and/or G2 galactosylated species of the antibody. In exemplary aspects, an increase of about 1 percent of β-galactose increases ADCP activity by about 2.5, about 2.8, about 2.88 or about 3 percent.

In an additional aspect, the disclosure provides a method of decreasing ADCP activity of trastuzumab comprising decreasing the amount of terminal β-galactose in the antibody or decreasing the amount of G1, G1a, G1b and/or G2 galactosylated species of the antibody. In certain instances, a decrease of about 1 percent of β-galactose decreases ADCP activity by about 2.5, about 2.8, about 2.88 or about 3 percent.

In a further aspect, the disclosure provides a method of matching the ADCP activity of a reference trastuzumab antibody comprising: (1) determining the ADCP activity of a reference trastuzumab antibody; (2) determining the ADCP activity of a second antibody having the same antibody sequence as the reference trastuzumab antibody; and (3) changing the ADCP activity of the second antibody by increasing or decreasing the amount of terminal β-galactose in the second antibody or increasing or decreasing the amount of G1, G1a, G1b and/or G2 galactosylated species of the second antibody, wherein the ADCP activity of the second antibody after increasing or decreasing the amount of terminal β-galactose or increasing or decreasing the amount of G1, G1a, G1b and/or G2 galactosylated species is the same as the reference antibody or within about 10%, about 15%, about 20%, about 25%, about 30% or about 35% of the reference antibody or within the range of about 1% to about 35% of the reference antibody. In exemplary instances, the ADCP activity of the second antibody is increased by increasing the amount of terminal β-galactose in the second antibody or increasing the amount of G1, G1a, G1b and/or G2 galactosylated species of the second antibody, wherein the ADCP activity of the second antibody after increasing ADCP activity is the same as the reference antibody or within about 10%, about 15%, about 20%, about 25%, about 30% or about 35% of the reference antibody or within the range of about 1% to about 35% of the reference antibody. Optionally, the ADCP activity of the second antibody is decreased by decreasing the amount of terminal β-galactose in the second antibody or decreasing the amount of G1, G1a, G1b and/or G2 galactosylated species of the second antibody, wherein the ADCP activity of the second antibody after decreasing ADCP activity is the same as the reference antibody or within about 10%, about 15%, about 20%, about 25%, about 30% or about 35% of the reference antibody or within the range of about 1% to about 35% of the reference antibody. In some embodiments, step 1 (“determining the ADCP activity of a reference trastuzumab antibody”) occurs before, after or at the same time as step 2 (“determining the ADCP activity of a second antibody having the same antibody sequence as the reference trastuzumab antibody”) and/or step 3 (“changing the ADCP activity of the second antibody . . . ”). In other embodiments, step 2 occurs before, after or at the same time as step 1 and/or step 3.

The disclosure also provides a method of engineering a specific target ADCP activity of a trastuzumab antibody comprising: (1) determining the ADCP activity of a trastuzumab antibody; (2) determining a target ADCP activity; and (3) changing the ADCP activity of the antibody by increasing or decreasing the amount of terminal β-galactose in the antibody or increasing or decreasing the amount of G1, G1a, G1b and/or G2 galactosylated species of the antibody, wherein the ADCP activity of the antibody after increasing or decreasing the amount of terminal β-galactose or increasing or decreasing the amount of G1, G1a, G1b and/or G2 galactosylated species is the same as the target ADCP activity or within about 10%, about 15%, about 20%, about 25%, about 30% or about 35% of the target ADCP activity or within about 1% to about 35% of the target ADCP activity. In some embodiments, step 1 (“determining the ADCP activity of a trastuzumab antibody”) occurs before, after or at the same time as step 2 (“determining a target ADCP activity”) and/or step 3 (“changing the ADCP activity of the antibody . . . ”). In some embodiments, step 2 (“determining a target ADCP activity”) occurs before, after or at the same time as step 1 (“determining the ADCP activity of a trastuzumab antibody”) and/or step 3 (“changing the ADCP activity of the antibody . . . ”).

The disclosure additionally provides a method of modulating ADCP activity of trastuzumab or rituximab comprising increasing or decreasing the amount of core fucose in the antibody; or increasing or decreasing the amount of afucosylated species of the antibody. The disclosure further provides a method of increasing ADCP activity of trastuzumab comprising decreasing the amount of core fucose in the antibody; or increasing the amount of afucosylated species of the antibody. Optionally, a decrease of about 1 percent of core fucose increases ADCP activity of the trastuzumab antibody by about 0.5, about 0.56, about 0.6 or about 1 percent.

The disclosure furthermore provides a method of increasing ADCP activity of rituximab comprising increasing the amount of core fucose in the antibody or decreasing the amount of afucosylated species of the antibody. In some instances, an increase of about 1 percent of core fucose increases ADCP activity of rituximab by about 0.5, about 0.7, about 0.75, about 0.8 or about 1 percent.

Provided by the disclosure is a method of decreasing ADCP activity of trastuzumab comprising increasing the amount of core fucose in the antibody or decreasing the amount of afucosylated species of the antibody. In certain aspects, an increase of about 1 percent of core fucose decreases ADCP activity of the trastuzumab antibody by about 0.5, about 0.56, about 0.6 or about 1 percent.

Also provided by the disclosure is a method of decreasing ADCP activity of rituximab comprising decreasing the amount of core fucose in the antibody or increasing the amount of afucosylated species of the antibody. Optionally, a decrease of about 1 percent of core fucose decreases ADCP activity of rituximab by about 0.5, about 0.7, about 0.75, about 0.8 or about 1 percent.

A method of matching ADCP activity of an IgG1 antibody is also provided by the present disclosure where the method comprises: (1) determining the ADCP activity of a reference IgG1 antibody; (2) determining the ADCP activity of a second IgG1 antibody having the same sequence as the reference IgG1 antibody; and (3) changing the ADCP activity of the second IgG1 antibody by increasing or decreasing the amount of core fucose in the second antibody or increasing or decreasing the amount of afucosylated species of the second antibody, wherein the ADCP activity of the second antibody after increasing or decreasing the amount of core fucose or increasing or decreasing the amount of afucosylated species is the same as the reference antibody or within about 10%, about 15%, about 20%, about 25%, about 30% or about 35% of the reference antibody or within the range of about 1% to about 35% of the reference antibody. In certain instances, the reference and second IgG1 antibodies are trastuzumab and (a) the ADCP activity of the second antibody is increased by decreasing the amount of core fucose or increasing the amount of afucosylated species of the second antibody; or (b) the ADCP activity of the second trastuzumab antibody is decreased by increasing the amount of core fucose or decreasing the amount of afucosylated species of the second antibody. Alternatively, the reference and second IgG1 antibodies are rituximab and (a) the ADCP activity of the second rituximab antibody is increased by increasing the amount of core fucose or decreasing the amount of afucosylated species of the second antibody; or (b) the ADCP activity of the second rituximab antibody is decreased by decreasing the amount of core fucose or increasing the amount of afucosylated species of the second antibody. In some embodiments, step 1 (“determining the ADCP activity of a reference IgG1 antibody”) occurs before, after or at the same time as step 2 (“determining the ADCP activity of a second IgG1 antibody having the same sequence as the reference IgG1 antibody”) and/or step 3 (“changing the ADCP activity of the second IgG1 antibody . . . ”). In some embodiments, step 2 occurs before, after or at the same time as step 1 and/or step 3.

A method of engineering a specific target ADCP activity of a trastuzumab antibody is also provided by the disclosure where the method comprises: (1) determining the ADCP activity of a trastuzumab antibody; (2) determining a target ADCP activity; and (3) changing the ADCP activity of the trastuzumab antibody by increasing or decreasing the amount of core fucose in the second antibody or increasing or decreasing the amount of afucosylated species of the second antibody, wherein the ADCP activity of the antibody after increasing or decreasing the amount of core fucose or increasing or decreasing the amount of afucosylated species is the same as the target ADCP activity or within about 10%, about 15%, about 20%, about 25%, about 30% or about 35% of the target ADCP activity or within the range of about 1% to about 35% of the target ADCP activity. In some embodiments, step 1 (“determining the ADCP activity of a trastuzumab antibody”) occurs before, after or at the same time as step 2 (“determining a target ADCP activity”) and/or step 3 (“changing the ADCP activity of the trastuzumab antibody . . . ”). In some embodiments, step 2 (“determining a target ADCP activity”) occurs before, after or at the same time as step 1 (“determining the ADCP activity of a trastuzumab antibody”) and/or step 3 (“changing the ADCP activity of the trastuzumab antibody . . . ”).

The disclosure additionally provides a method of engineering a specific target ADCP activity of a rituximab antibody comprising: (1) determining the ADCP activity of a rituximab antibody; (2) determining a target ADCP activity; and (3) changing the ADCP activity of the rituximab antibody by increasing or decreasing the amount of core fucose in the second antibody or increasing or decreasing the amount of afucosylated species of the second antibody, wherein the ADCP activity of the antibody after increasing or decreasing the amount of core fucose or increasing or decreasing the amount of afucosylated species is the same as the target ADCP activity or within about 10%, about 15%, about 20%, about 25%, about 30% or about 35% of the target ADCP activity or within the range of about 1% to about 35% of the target ADCP activity. In some embodiments, step 1 (“determining the ADCP activity of a rituximab antibody”) occurs before, after or at the same time as step 2 (“determining a target ADCP activity”) and/or step 3 (“changing the ADCP activity of the rituximab antibody . . . ”). In some embodiments, step 2 (“determining a target ADCP activity”) occurs before, after or at the same time as step 1 (“determining the ADCP activity of a rituximab antibody”) and/or step 3 (“changing the ADCP activity of the rituximab antibody . . . ”).

The disclosure also provides a method of modulating ADCP activity of an IgG1 antibody (such as trastuzumab or rituximab) comprising increasing or decreasing the amount of high mannose in the antibody or increasing or decreasing the amount of M5 high mannose species of the antibody.

A method of increasing ADCP activity of an IgG1 antibody (such as trastuzumab or rituximab) is also provided by the disclosure where the method comprises decreasing the amount of high mannose in the antibody or decreasing the amount of M5 high mannose species of the antibody. In some embodiments, a decrease of about 1 percent of high mannose increases ADCP activity by about 1, about 1.2, about 1.31, about 1.5, about 1.7, about 2, about 2.11 or about 2.5 percent.

The disclosure provides a method of decreasing ADCP activity of an IgG1 antibody (such as trastuzumab or rituximab) comprising increasing the amount of high mannose in the antibody or increasing the amount of M5 high mannose species of the antibody. In some embodiments, an increase of about 1 percent of high mannose decreases ADCP activity by about 1, about 1.2, about 1.31, about 1.5, about 1.7, about 2, about 2.11 or about 2.5 percent.

Also provided is a method of matching ADCP activity of an IgG1 antibody (such as trastuzumab or rituximab) comprising: (1) determining the ADCP activity of a reference IgG1 antibody; (2) determining the ADCP activity of a second IgG1 antibody having the same sequence as the reference IgG1 antibody; and (3) changing the ADCP activity of the second IgG1 antibody by increasing or decreasing the amount of high mannose in the second antibody or increasing or decreasing the amount of M5 high mannose species of the second antibody, wherein the ADCP activity of the second antibody after increasing or decreasing the amount of high mannose or the amount of M5 high mannose species is the same as the reference antibody or within about 10%, about 15%, about 20%, about 25%, about 30% or about 35% of the reference antibody or within the range of about 1% to about 35% of the reference antibody. In some aspects, the ADCP activity of the second IgG1 antibody is increased by decreasing the amount of high mannose or decreasing the amount of M5 high mannose species in the second antibody. In exemplary instances, the ADCP activity of the second IgG1 antibody is decreased by increasing the amount of high mannose or increasing the amount of M5 high mannose species in the second antibody. In some embodiments, step 1 (“determining the ADCP activity of a reference IgG1 antibody”) occurs before, after or at the same time as step 2 (“determining the ADCP activity of a second IgG1 antibody . . . ”) and/or step 3 (“changing the ADCP activity of the second IgG1 antibody . . . ”). In some embodiments, step 2 occurs before, after or at the same time as step 1 and/or step 3.

In another aspect, the disclosure provides a method of engineering a specific target ADCP activity of an IgG1 antibody (such as trastuzumab or rituximab) comprising: (1) determining the ADCP activity of a IgG1 antibody; (2) determining a target ADCP activity; and (3) changing the ADCP activity of the IgG1 antibody by increasing or decreasing the amount of high mannose in the second antibody or increasing or decreasing the amount of M5 high mannose species in the second antibody, wherein the ADCP activity of the antibody after increasing or decreasing the amount of high mannose or the amount of M5 high mannose species is the same as the target ADCP activity or within about 10%, about 15%, about 20%, about 25%, about 30% or about 35% of the target ADCP activity or within the range of about 1% to about 35% of the target ADCP activity. In some embodiments, step 1 (“determining the ADCP activity of a IgG1 antibody”) occurs before, after or at the same time as step 2 (“determining a target ADCP activity”) and/or step 3 (“changing the ADCP activity of the IgG1 antibody . . . ”). In some embodiments, step 2 (“determining a target ADCP activity”) occurs before, after or at the same time as step 1 (“determining the ADCP activity of a IgG1 antibody”) and/or step 3 (“changing the ADCP activity of the IgG1 antibody . . . ”).

Also provided by the disclosure is a method of modulating ADCP activity of an IgG1 antibody (such as trastuzumab or rituximab) comprising increasing or decreasing: (1) the amount of terminal β-galactose and/or the amount of G1, G1a, G1b and/or G2 galactosylated species in the antibody; (2) the amount of core fucose and/or the amount of afucosylated species in the antibody; and/or (3) the amount of high mannose or the amount of M5 high mannose species in the antibody. Additionally provided is a method of increasing ADCP activity of an IgG1 antibody (such as trastuzumab or rituximab) comprising: (1) increasing the amount of terminal β-galactose or the amount of G1, G1a, G1b and/or G2 galactosylated species in the antibody; (2) decreasing the amount of core fucose or increasing the amount of afucosylated species in the antibody; and/or (3) decreasing the amount high mannose or decreasing the amount of M5 high mannose species in the antibody. In other exemplary embodiments, the disclosure provides a method of decreasing ADCP activity of an IgG1 antibody (such as trastuzumab or rituximab) comprising: (1) decreasing the amount of terminal β-galactose or the amount of G1, G1a, G1b and/or G2 galactosylated species in the antibody; (2) increasing the amount of core fucose or decreasing the amount of afucosylated species in the antibody; and/or (3) increasing the amount high mannose or the amount of M5 high mannose species in the antibody.

In exemplary instances of the methods of the disclosure, (1) the amount of terminal β-galactose or the amount of G1, G1a, G1b and/or G2 galactosylated species; (2) the amount of core fucose or the amount of afucosylated species; and/or (3) the amount of high mannose or the amount of M5 high mannose species is increased or decreased by culturing cells expressing the antibody in cell culture media or under cell culture conditions that modulates the amount of one or more of these glycans (e.g., terminal β-galactose; G1, G1a, G1b and/or G2 galactosylated species; core fucose; afucosylated species; high mannose and/or M5 high mannose species). In some embodiments, the amount of these glycans (terminal β-galactose; G1, G1a, G1b and/or G2 galactosylated species; core fucose; afucosylated species; high mannose and/or M5 high mannose species) is increased or decreased using chemical means or an enzyme, such as EndoS; Endo-S2; Endo-D; Endo-M; endoLL; α-fucosidase; β-(1-4)-Galactosidase; Endo-H; β-1,4-galactosyltransferase; and/or PNGase F. In certain aspects, the amount of these glycans is modulated by incubating the antibody with β-1,4-galactosyltransferase for about 10 minutes, for about 20 minutes, for about 30 minutes, for about 1 hour, for about 2 hours, for about 4 hours, for about 9 hours, or for a period of time falling with the range of about 10 minutes to about 9 hours.

The disclosure also provides an antibody composition produced by any one of the methods of the disclosure. In certain aspects, the antibody has increased or decreased ADCP activity compared to a reference IgG1 antibody having the same antibody sequence as the IgG1 antibody having increased or decreased ADCP activity. In some instances, the antibody has about 30%, about 35%, about 40%, about 45%, about 50%, about 55%, about 60%, about 65%, about 70%, about 75%, about 80%, about 85%, about 90%, about 95%, about 100%, about 125%, about 150%, about 175% or about 200%, or about 1-fold, about 2-fold, about 3-fold, about 4-fold, about 5-fold, about 6-fold, about 7-fold, about 8-fold, about 9-fold, or about 10-fold increased ADCP activity compared to the reference antibody, or about 0.5-fold to about 8-fold increased ADCP activity compared to the reference antibody. Optionally, the antibody has about 30%, about 35%, about 40%, about 45%, about 50%, about 55%, about 60%, about 65%, about 70%, about 75%, about 80%, about 85%, about 90%, about 95%, about 100%, about 125%, about 150%, about 175% or about 200% or about 1-fold, about 2-fold, about 3-fold, about 4-fold, about 5-fold, about 6-fold, about 7-fold, about 8-fold, about 9-fold, or about 10-fold decreased ADCP activity compared to the reference antibody, or about 0.5-fold to about 8-fold decreased ADCP activity compared to the reference antibody. In exemplary aspects, the IgG1 antibody is trastuzumab or rituximab comprising any one of the sequences recited in Tables 1 or 2.

The disclosure further provides pharmaceutical compositions comprising any one of the antibody compositions described herein.

### BRIEF DESCRIPTION OF THE DRAWINGS

FIG. 1A is an illustration of the three types of N-glycans (oligomannose, complex and hybrid) and commonly used symbols for such saccharides.

FIG. 1B is a cartoon illustrating the major N-Linked glycans found in human IgGs at the N-glycosylation site asparagine (Asn) 297 with a representative attachment of an oligosaccharide structure. These glycans are commonly composed of a core heptasaccharide and outer arms constructed by variable addition of fucose, N-acetylglucosamine (GlcNAc), galactose, sialic acid (SA), and bisecting N-GlcNAc.

FIG. 1C is a diagram of the salvage pathway and the de novo pathway of fucose metabolism. In the salvage pathway, free L-fucose is converted to GDP-fucose, while in the de novo pathway, GDP-fucose is synthesized via three reactions catalyzed by GMD and FX. GDP-fucose is then transported from the cytosol to the Golgi lumen by GDP-Fuc Transferase and transferred to acceptor oligosaccharides and proteins. The other reaction product, GDP, is converted by a luminal nucleotide diphosphatase to guanosine 5-monophosphate (GMP) and inorganic phosphate (Pi). The former is exported to the cytosol (via an antiport system that is coupled with the transport of GDP-fucose), whereas the latter is postulated to leave the Golgi lumen via the Golgi anion channel, GOLAC. See, e.g., Nordeen et al. 2000; Hirschberg et al. 2001.

FIG. 2 is a dose-response curve (smooth fit) for three different IgG1 mAbs targeting HER2 (trastuzumab), TNFα, or CD20 (rituximab) cell surface antigens in an ADCP reporter gene assay showing that the tested antibodies displayed a range of activities in this ADCP assay. The anti-TNFα antibody showed virtually no response, while the anti-CD20 antibody showed very robust response and the anti-HER2 antibody showed an intermediate response.

FIGS. 3A-D are graphs showing linearity and range assessment for the anti-CD20 antibody rituximab (FIGS. 3A-B) and for anti-HER2 antibody trastuzumab (FIGS. 3C-D). A linear regression line (with the equations shown) was fit for a plot of the known vs. measured ADCP activity for each antibody (FIGS. 3A & 3C), with representative dose-response curves provided for the various activity levels within the correlated line graphs (FIGS. 3B & 3D). This data demonstrated the quantitative nature and range of the ADCP reporter gene assay, and its suitability for the assessment of quality attributes that impact ADCP activity.

FIG. 4 is a summary of the structures of the three major glycan species evaluated in the ADCP reporter gene assay, including afucosylated species (i.e. species lacking core-fucose, including G0 or G1), high mannose species (including M5 species) or terminal β-galactose species (i.e. terminal beta-galactose, including G1F or G2F) species.

FIG. 5 is a representative ADCP dose response curve for anti-HER2 antibodies (trastuzumab) having widely ranging β-galactose levels showing that higher levels of β-galactose levels generally result in higher ADCP activity.

FIG. 6 is a line graph showing the correlation between ADCP activity and β-galactose levels for the anti-HER2 antibody trastuzumab. The relative impact of β-galactose on ADCP activity of trastuzumab was calculated as 2.88.

FIG. 7 is a bar graph reporting the relative ADCP activity for an anti-CD20 antibody (rituximab) for two wide ranging β-galactose levels (0% and 81% β-galactose).

FIG. 8 is a line graph reporting the correlation between FcγRIIa binding and β-galactose levels for the anti-HER2 antibody trastuzumab as measured by a FcγRIIa binding assay using an AlphaLISA format.

FIGS. 9A-B are bar graphs reporting the relative target biding activity for select β-galactose samples as shown, normalized to the activity of the lowest β-galactose levels for CD20/rituximab (FIG. 9A) and HER2/trastuzumab (FIG. 9B) antibodies. These results demonstrate that the observed differences in relative ADCP activity is due to differences in FcγRIIa binding and not to inadvertent changes in cell surface target binding.

FIGS. 10A-B are line graphs reporting the correlation between ADCP activity and afucosylation for the anti-HER2 antibody trastuzumab (FIG. 10A) and the anti-CD20 antibody rituximab (FIG. 10B). This data shows a linear increase in ADCP activity when increasing afucosylation of trastuzumab with a response coefficient of 0.56. Rituximab showed a modest linear decrease in ADCP activity when increasing afucosylation.

FIGS. 11A-B are line graphs reporting the correlation of β-galactose and ADCP activity (FIG. 11A) or the correlation of β-galactose and FcγRIIa binding (FIG. 11B) for trastuzumab with both fixed high mannose containing samples and samples that contain a range of high mannose. This data suggests that increasing levels of high mannose decrease ADCP activity and FcγRIIa binding of trastuzumab.

FIGS. 12A-B are line graphs reporting the correlation of high mannose and ADCP activity (FIG. 12A) or the correlation of high mannose and FcγRIIa binding (FIG. 12B) for the rituximab anti-CD20 antibody. This data suggests that increasing levels of high mannose decreases ADCP activity and FcγRIIa binding of rituximab.

### DETAILED DESCRIPTION

In order that the present disclosure can be more readily understood, certain terms are first defined. Additional definitions are set forth throughout the detailed description.

As used herein, the terms “a,” “an,” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,” and permit the presence of one or more features or components) unless otherwise noted. The terms “a” (or “an”), as well as the terms “one or more,” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” as used in connection with a numerical value throughout the specification and the claims denotes an interval of accuracy, familiar and acceptable to a person skilled in the art. In general, such interval of accuracy is ±10%.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure. Generally, nomenclatures used in connection with, and techniques of, cell and tissue culture, molecular biology, immunology, protein glycosylation, antibody production and antibody purification, described herein are those well-known and commonly used in the art. Standard techniques may be used for recombinant DNA, oligonucleotide synthesis, tissue culture and transformation, protein purification, antibody generation, etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manuel, 3rd ed., Cold Spring Harbor Laboratory Press, cold Spring Harbor, N.Y., which is incorporated herein by reference for any purpose.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole.

### Post-Translational GLYCOSYLATION

Many secreted proteins undergo post-translational glycosylation, a process by which sugar moieties (e.g., glycans, saccharides) are covalently attached to specific amino acids of a protein. In eukaryotic cells, two types of glycosylation reactions occur: (1) N-linked glycosylation, in which glycans are attached to the asparagine of the recognition sequence Asn-X-Thr/Ser, where “X” is any amino acid except proline, and (2) O-linked glycosylation in which glycans are attached to serine or threonine. Regardless of the glycosylation type (N-linked or O-linked), microheterogeneity of protein glycoforms exists due to the large range of glycan structures associated with each site (O or N).

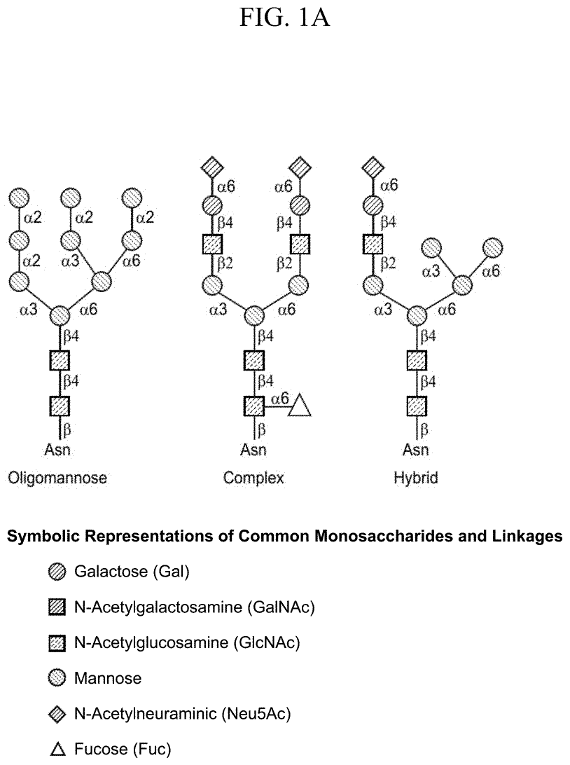

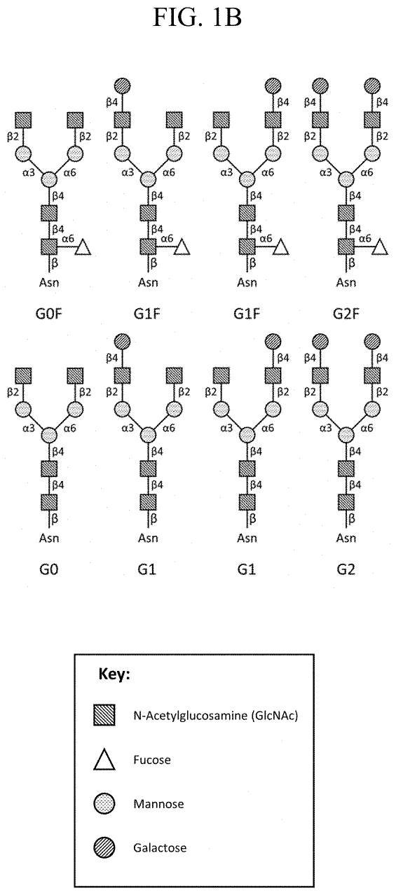

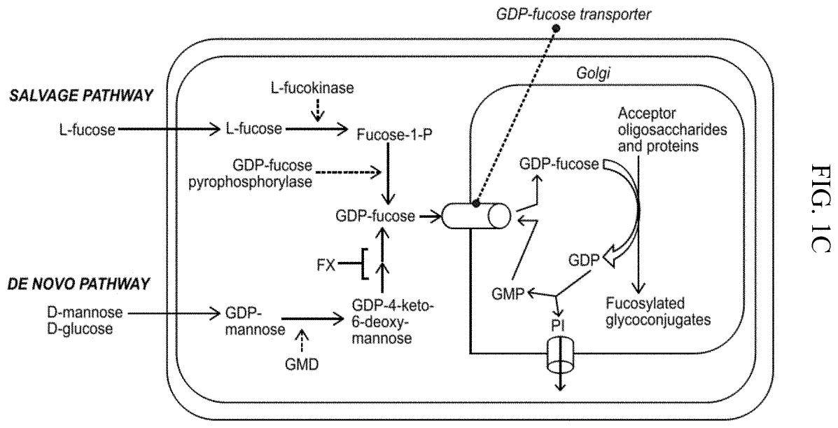

All N-glycans have a common core sugar sequence: Manα1-6(Manα1-3)Manβ1-4GlcNAcβ1-4GlcNAcβ1-Asn-X-Ser/Thr (Man₃GlcNAc₂Asn) and are categorized into one of three types: (A) a high mannose (HM) or oligomannose (OM) type, which consists of two N-acetylglucosamine (GalNAc) moieties and a large number (e.g., 5, 6, 7, 8 or 9) of mannose (Man) residues (B) a complex type, which comprises more than two GlcNAc moieties and any number of other sugar types or (C) a hybrid type, which comprises a Man residue on one side of the branch and GlcNAc at the base of a complex branch. FIG. 1A (based on Stanley et al., Chapter 8: N-Glycans, Essentials of Glycobiology, 2^{nd} ed., Cold Spring Harbor Laboratory Press; 2009) shows the three types of N-glycans.

N-linked glycans typically comprise one or more monosaccharides of galactose (Gal), N-acetylgalactosamine (GalNAc), N-acetylglucoasamine (GlcNAc), mannose (Man), N0Acetylneuraminic acid (Neu5Ac), fucose (Fuc). The commonly used symbols for such saccharides are shown in FIG. 1A.

N-linked glycosylation begins in the endoplasmic reticulum (ER), where a complex set of reactions result in the attachment of a core glycan structure made essentially of two GlcNAc residues and three Man residues. The glycan complex formed in the ER is modified by action of enzymes in the Golgi apparatus. If the saccharide is relatively inaccessible to the enzymes, it typically stays in the original HM form. If enzymes can access the saccharide, then many of the Man residues are cleaved off and the saccharide is further modified, resulting in the complex type N-glycans structure. For example, mannosidase-1 located in the cis-Golgi, can cleave or hydrolyze a HM glycan, while fucosyltransferase FUT-8, located in the medial-Golgi, fucosylates the glycan (Hanrue Imai-Nishiya (2007), BMC Biotechnology, 7:84).

Accordingly, the sugar composition and the structural configuration of a glycan structure varies, depending on the glycosylation machinery in the ER and the Golgi apparatus, the accessibility of the machinery enzymes to the glycan structure, the order of action of each enzyme and the stage at which the protein is released from the glycosylation machinery, among other factors.

Controlling the glycan structure is important in recombinant production of therapeutic monoclonal antibodies, as the glycan structure attached to the Fc domain influences the interaction with the FcγRs that mediate ADCC and ADCP and with C1q binding, the initial binding event leading to CDC.

The present disclosure identifies the impact of various glycans (including, e.g., (3-galactose, core-fucose and/or high mannose) on ADCP activity of IgG1 antibodies, including trastuzumab or rituximab. Accordingly, the present disclosure provides a method of modulating ADCP activity of an antibody, or a composition comprising the same (an antibody composition). In exemplary embodiments, the method comprises modulating the amount of (a) galactosylated glycans of the antibody; (b) afucosylated glycans of the antibody; (c) high mannose glycans of the antibody; or (d) a combination thereof. Without being bound to a particular theory, it is believed that the methods of the disclosure provide a means for tailor-made compositions comprising specific amounts of particular glycoforms of a given antibody, which exhibit targeted levels of ADCP activity.

The term “Antibody Dependent Cellular Phagocytosis” or “ADCP” refers to the mechanism by which antibody-opsonized target cells activate the FcγRs on the surface of cell of the immune system (including, e.g., macrophages and/or neutrophils) to induce phagocytosis, resulting in the internalization and degradation of the target cell through acidification of the phagosome. ADCP generally requires a two-step mechanism—(1) binding of the antibody to an antigen or target on a cell and (2) binding of the antibody to receptors (Fc Receptors) on a macrophage or other phagocytic cells to induce internalization, degradation and/or destruction of the antibody-bound cell. See, e.g., Gül N and Egmond M. Antibody-Dependent Phagocytosis of Tumor Cells by Macrophages: A Potent Effector Mechanism of Monoclonal Antibody Therapy of Cancer. Cancer Res Dec. 1, 2015 (75) (23) 5008-5013.

The term “FcγR” or “Fc-gamma receptor” is a protein belonging to the IgG superfamily involved in inducing phagocytosis of opsonized cells or microbes. See, e.g., Fridman W H. Fc receptors and immunoglobulin binding factors. FASEB Journal. 5 (12): 2684-90 (1991). Members of the Fc-gamma receptor family include: FcγRI (CD64), FcγRIIA (CD32), FcγRIIB (CD32), FcγRIIIA (CD16a), and FcγRIIIB (CD16b). The sequences of FcγRI, FcγRIIA, FcγRIIB, FcγRIIIA, and FcγRIIIB can be found in many sequence databases, for example, at the Uniprot database (www.uniprot.org) under accession numbers P12314 (FCGR1_HUMAN), P12318 (FCG2A_HUMAN), P31994 (FCG2B_HUMAN), P08637 (FCG3A_HUMAN), and P08637 (FCG3A_HUMAN), respectively.

The term “ADCP activity” refers to the extent to which phagocytosis, resulting in the internalization and degradation of a target cell through acidification of the phagosome, is activated or stimulated upon the two-step mechanism of (1) binding of the antibody to an antigen or target on a cell and (2) binding of the antibody to receptors (Fc Receptors) on a macrophage or other phagocytic cells to induce internalization, degradation and/or destruction of the antibody-bound cell. The phrase “ADCP activity of an antibody” refers to the ability of an antibody to induce ADCP.

Methods of measuring or determining the ADCP activity of an antibody, including commercially available assays and kits, are well-known in the art, as described, e.g., in Richards, J. O. et al. Optimization of antibody binding to FcγRIIa enhances macrophage phagocytosis of tumor cells. Mol. Cancer Ther. 2008; 7, 2517-27; Ackerman et. al, A robust, high-throughput assay to determine the phagocytic activity of clinical antibody samples. Journal of Immunological Methods 366 (2011) 8-19; Chung S, et al. Quantitative evaluation of fucose reducing effects in a humanized antibody on Fcgamma receptor binding and antibody-dependent cell-mediated cytotoxicity activities. mAbs 2012; 4:326-40; Herbrand U. Antibody-dependent cellular phagocytosis: the mechanism of action that gets no respect—A discussion about improving bioassay reproducibility. BioProcess J, 2016; 15(1): 26-9; ADCP Bioassays (Promega, catalog number G9901 or G9991); and ADCP Bioassay (BPS Bioscience, catalog number 60540 or 60541), all herein incorporated by reference for all purposes. The term “ADCP Assay” or “FcγR reporter gene assay” refers to an assay, kit or method useful to determine the ADCP activity of an antibody.

Exemplary methods of measuring or determining the ADCP activity of an antibody in the methods described herein include the ADCP assay described in the Examples or the ADCP Reporter Assay commercially available from Promega (Catalog No. G9901 or G9991). In some embodiments, ADCP activity is measured or determined using reporter gene assay, e.g., a FcγR reporter gene assay containing one or more of the following: a Jurkat cell expressing a FcγR receptor (including, e.g., FcγRIIa), a nuclear factor of activated T cell (NFAT)-response element and Luciferase or other reporter gene or reporter product. In exemplary aspects, ADCP activity is measured using a reporter gene assay comprising cells, e.g., Jurkat T cells, expressing FcγRIIa on the cell surface and a reporter gene linked to NFAT activity.

### Modulating ADCP Activity

The term “modulate” or “modulating” means to change by increasing or decreasing. Thus, the term “modulating” as used in a phrase such as “modulating Antibody Dependent Cellular Phagocytosis activity” herein is intended to include increasing Antibody Dependent Cellular Phagocytosis activity or decreasing Antibody Dependent Cellular Phagocytosis activity. Also, the term “modulating” as used in a phrase such as “modulating the amount of galactosylated glycans, afucosylated glycans, high mannose glycans, or a combination thereof” is intended to include increasing the amount of said glycans or decreasing the amount of said glycans.

Accordingly, in exemplary embodiments, the presently disclosed method represents a method of increasing ADCP activity of an antibody or a composition comprising the same. In exemplary aspects, the methods of the present disclosure increase the ADCP activity of the antibody, or composition comprising the same, to any degree or level relative to a control or a reference antibody. In exemplary instances, the increase provided by the methods of the disclosure is at least or about a 1% to about a 100% increase (e.g., at least or about a 1% increase, at least or about a 2% increase, at least or about a 3% increase, at least or about a 4% increase, at least or about a 5% increase, at least or about a 6% increase, at least or about a 7% increase, at least or about a 8% increase, at least or about a 9% increase, at least or about a 9.5% increase, at least or about a 9.8% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, at least or about a 100% increase) relative to a control or a reference antibody. In exemplary embodiments, the increase provided by the methods of the disclosure is over 100%, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to a control or a reference antibody. In exemplary embodiments, the level of ADCP activity of the antibody or composition comprising the same increases by at least about 1.5-fold, relative to a control or a reference antibody. In exemplary embodiments, the level of ADCP activity of the antibody or composition comprising the same increases by at least about 2-fold, relative to a control or a reference antibody. In exemplary embodiments, the level of ADCP activity of the antibody or composition comprising the same increases by at least about 3-fold, relative to a control or a reference antibody. In exemplary embodiments, the level of ADCP activity of the antibody or composition comprising the same increases by at least about 4-fold or about 5-fold, relative to a control or a reference antibody. In exemplary embodiments, the level of ADCP activity of the antibody or composition comprising the same increases by at least about 6-fold, about 7-fold, about 8-fold, about 9-fold, or about 10-fold, relative to a control or a reference antibody. In exemplary embodiments, the level of ADCP activity of the antibody or composition comprising the same increases by an amount falling within the range of about 0.5-fold to about 8-fold, relative to a control or a reference antibody.

In alternative embodiments, the presently disclosed method represents a method of decreasing ADCP activity of an antibody or a composition comprising the same. In some aspects, the methods of the disclosure decrease the level of ADCP activity of the antibody, or composition comprising the same, to any degree or level relative to a control or a reference antibody. For example, the decrease provided by the methods of the disclosure is at least or about a 1% to about a 100% decrease (e.g., at least or about a 1% decrease, at least or about a 2% decrease, at least or about a 3% decrease, at least or about a 4% decrease, at least or about a 5% decrease, at least or about a 6% decrease, at least or about a 7% decrease, at least or about a 8% decrease, at least or about a 9% decrease, at least or about a 9.5% decrease, at least or about a 9.8% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, at least or about a 100% decrease) relative to the level of a control or a reference antibody. In exemplary embodiments, the decrease provided by the methods of the disclosure is over about 100%, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to the level of a control or a reference antibody. In exemplary embodiments, the level of ADCP activity of the antibody, or composition comprising the same decreases by at least or about 1.5-fold, relative to a control or a reference antibody. In exemplary embodiments, the level of ADCP activity of the antibody, or composition comprising the same decreases by at least about 2-fold, relative to a control or a reference antibody. In exemplary embodiments, the level of ADCP activity of the antibody, or composition comprising the same decreases by at least about 3-fold, relative to a control or a reference antibody. In exemplary embodiments, the level of ADCP activity of the antibody, or composition comprising the same decreases by at least about 4-fold or by at least about 5-fold, relative to a control or a reference antibody. In exemplary embodiments, the level of ADCP activity of the antibody or composition comprising the same decreases by at least about 6-fold, about 7-fold, about 8-fold, about 9-fold, or about 10-fold, relative to a control or a reference antibody. In exemplary embodiments, the level of ADCP activity of the antibody or composition comprising the same decreases by an amount falling within the range of about 0.5-fold to about 8-fold, relative to a control or a reference antibody.

### Glycans

In exemplary embodiments, the methods disclosed herein comprises modulating the amount of glycans on an antibody including: (a) galactosylated glycans; (b) afucosylated glycans; (c) high mannose glycans; or (d) a combination thereof to increase or decrease ADCP activity of the antibody. In exemplary aspects, the methods disclosed herein comprises modulating the amount of glycans attached to the Fc domain of an antibody including: (a) galactosylated glycans; (b) afucosylated glycans; (c) high mannose glycans; or (d) a combination thereof to increase or decrease ADCP activity of the antibody. In additional exemplary aspects, the methods disclosed herein comprises modulating the amount of glycans attached to Asn-297 of the Fc domain at of an antibody including: (a) galactosylated glycans; (b) afucosylated glycans; (c) high mannose glycans; or (d) a combination thereof to increase or decrease ADCP activity of the antibody.

In exemplary aspects, the methods provided by the present disclosure relate to modulation of an antibody composition, e.g., an IgG1 antibody composition (including, e.g., trastuzumab or rituximab), wherein steps are taken to achieve a desired or predetermined or pre-selected level of glycoforms of the IgG1 antibody to achieve a desired or predetermined or pre-selected level of ADCP activity. In exemplary embodiments, the method comprises modulating (increasing or decreasing) the amount of (a) galactosylated glycans; (b) afucosylated glycans; (c) high mannose glycans; or (d) a combination thereof of the IgG1 antibody to modulate (increase or decrease) the ADCP activity induced or stimulated by the antibody composition. In exemplary embodiments, the method comprises modulating (increasing or decreasing) the amount of glycoforms, e.g., (a) galactosylated glycoforms; (b) afucosylated glycoforms; (c) high mannose glycoforms; or (d) a combination thereof, to modulate (increase or decrease) the ADCP activity induced or stimulated by the antibody composition. Without being bound to a particular theory, it is believed that the methods of the disclosure provide a means for engineering antibody compositions having a desired, predetermined or target ADCP activity by modulating (i.e. increasing or decreasing) the amounts of particular glycoforms of a given antibody. Accordingly, in some embodiments, the methods disclosed herein comprise modulating the amount or percentage of (a) galactosylated glycans; (b) afucosylated glycans; (c) high mannose glycans; or (d) a combination thereof within an antibody composition to achieve a composition having a desired, predetermined or target ADCP activity and/or an increased or decreased ADCP activity.

In alternative aspects, the methods disclosed herein comprise modulating the amount of terminal β-galactose, core fucose, or high mannose, or a combination thereof, attached to a particular IgG1 molecule (including, e.g., trastuzumab or rituximab). For example, the method may comprise increasing the amount of terminal β-galactose on trastuzumab (by, e.g., effectively changing the glycan from a G0 species to a G1 or G2 species or from a G1 species to a G2 species) to increase ADCP activity of the trastuzumab antibody. Also, for example, the method may comprise decreasing the amount of terminal β-galactose on trastuzumab (by, e.g., effectively changing the glycan from a G2 species to a G1 or G0 species or from a G1 species to a G0 species) to decrease ADCP activity of the trastuzumab antibody. In other exemplary aspects, the method may comprise decreasing the amount of core fucose or increasing the amount of afucosylated species in a trastuzumab antibody to increase ADCP activity. In other exemplary aspects, the method may comprise increasing the amount of core fucose or decreasing the amount of afucosylated species in a rituximab antibody to increase ADCP activity of the antibody. In other exemplary aspects, the method may comprise increasing the amount of core fucose or decreasing the amount of afucosylated species in a trastuzumab antibody to decrease ADCP activity. In other exemplary aspects, the method may comprise decreasing the amount of core fucose or increasing the amount of afucosylated species in a rituximab antibody to decrease ADCP activity of the antibody. In further exemplary aspects, the method may comprise increasing the amount of high mannose or M5 high mannose on an IgG1 antibody (such as trastuzumab or rituximab) to decrease ADCP activity of the antibody or decreasing the amount of mannose or M5 high mannose on an IgG1 antibody (such as trastuzumab or rituximab) to increase ADCP activity of the antibody.

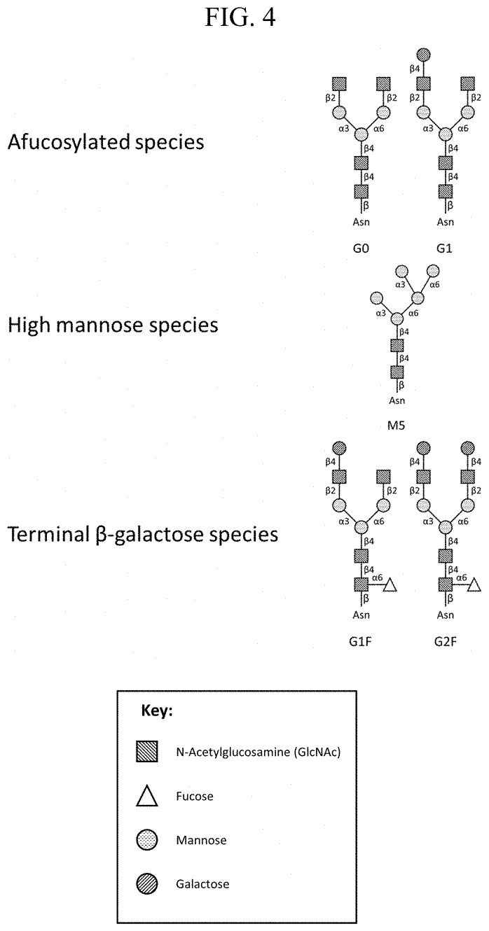

The term “glycan”, “glycans”, “glycoform” or “glycoforms” refers to oligomers of monosaccharide species that are connected by various glycosidic bonds. Examples of monosaccharides commonly found in mammalian N-linked glycans include hexose (Hex), glucose (Glc), galactose (Gal), mannose (Man) and N-acetylglucosamine (GlcNAc). The major N-glycan species found on recombinant IgG1 antibodies include fucose, galactose, mannose, sialic acid and GlcNAc, as depicted in FIG. 1B. The glycan oligosaccharide structures are linked to the N-glycosylation site at Asn-297 and are generally composed of a core heptasaccharide with outer arms constructed by variable addition of fucose, N-acetylglucosamine (GlcNAc), galactose, sialic acid (SA), and bisecting N-GlcNAc. Each of the potential oligosaccharide structures may be abbreviated as follows: G0, G1, or G2 referring to the core GlcNAc and mannose oligosaccharide structure having zero, one or two terminal galactose molecules, respectively. Within G1, two additional structures, abbreviated G1a and G1b, may be present with G1a or G1b referring to whether the terminal galactose group is attached to either the 6-arm or the 3-arm of the core structure. See FIG. 1B. When fucosylated (i.e. a fucose group is attached to the core glycan structure) the G0, G1 (G1a/G1b) or G2 forms may be abbreviated G0F, G1F (G1aF/G1bF) or G2F. When sialic acid is present, these abbreviations contain a “S” such that, for example, G2FS2 refers to a glycan having two galactose, a fucose and two sialic acid groups. Additional glycans linked to IgG1 antibodies may also exist including high mannose (HM) structures, which are formed by the incorporation of additional mannose groups, including the high mannose species “M5” as shown in FIG. 4. As used herein, the term “glycan” or “glycans” refers to any of the oligomers of monosaccharide species described herein or any other oligomers of monosaccharaide species linked to an antibody or an IgG1 antibody.

The “terminal β-galactose, “galactosylated glycans” or “G1, G1a, G1b and/or G2 galactosylated species” refers to a glycan comprising one or two galactose molecules linked to an IgG1 antibody at the N-glycosylation site (Asn-297) through the N-acetylglucoseamine moieties that attach to the core mannose structure. Exemplary glycans comprising “terminal β-galactose” “galactosylated glycans” or “G1, G1a, G1b and/or G2 galactosylated species” are depicted in FIG. 1B. In some embodiments, the G1, G1a, G1b and/or G2 galactosylated species may or may not contain core fucose.

The term “core fucose” or “fucosylated species” refers to a glycan comprising a fucose molecule (alpha 1-6) linked to an IgG1 antibody at the N-glycosylation site (Asn-297) through the N-acetylglucoseamine moieties that attach to the core mannose structure. Exemplary glycans comprising “core fucose” or “fucosylated species” are depicted in FIG. 1B. In some embodiments, antibodies containing core fucose and/or a fucosylated species may or may not contain other glycans including terminal β-galactose and/or high mannose.

The term “afucosylated”, “afucosylated glycans” or “afucosylation” refers to the removal or lack of a core fucose on an antibody. Exemplary afucosylated antibody species are depicted in FIG. 1B. In some embodiments, antibodies lacking core fucose may or may not contain other glycans including terminal β-galactose and/or high mannose. Afucosylated glycoforms include, but are not limited to, A1G0, A1G1a, A2G0, A2G1a, A2G1b, A2G2, and A1G1M5. See, e.g., Reusch and Tejada, Glycobiology 25(12): 1325-1334 (2015).

The term “high mannose”, “high mannose glycans” or “HM” refers to a glycan comprising more than 3 mannose molecules linked to an IgG1 antibody at the N-glycosylation site (Asn-297). Exemplary high mannose antibodies are depicted in FIG. 4, including the “M5 high mannose antibody species” which contains two additional mannose molecules. High mannose glycans encompass glycans comprising 5, 6, 7, 8, or 9 mannose residues, abbreviated as Man5, Man6, Man7, Man8, and Man9, respectively

The term “amount” when referring the amount of a glycan (including, e.g., (1) the amount of terminal β-galactose, (2) the amount of G1, G1a, G1b and/or G2 galactosylated species, (3) the amount of core fucose, (4) the amount of afucosylated species, (5) the amount of high mannose, and/or (6) the amount of M5 high mannose species) refers to a relative amount or percentage of a particular glycan compared to the total amount of glycans. For example, the amount of (1) terminal β-galactose, (2) G1, G1a, G1b and/or G2 galactosylated species, (3) core fucose, (4) afucosylated species, (5) high mannose, and/or (6) M5 high mannose species is denoted as a percentage calculated as the amount of terminal β-galactose, G1, G1a, G1b and/or G2 galactosylated species, core fucose, afucosylated species, high mannose, or M5 high mannose species divided by the total amount of all glycans. Methods for measuring and determining the amount or relative percentage of a glycan (including, e.g., terminal β-galactose, G1, G1a, G1b and/or G2 galactosylated species, core fucose, afucosylated species, high mannose, and/or M5 high mannose species) are well known in the art and include Hydrophilic Interaction Liquid Chromatography (HILIC)) as described in the Examples. See also, Pace et al., Characterizing the Effect of Multiple Fc Glycan Attributes on the Effector Functions and FccRIlla Receptor Binding Activity of an IgG1 Antibody, Biotechnol. Prog., 2016, Vol. 32, No. 5 pages 1181-1192; and Shah, B. et al. LC-MS/MS Peptide Mapping with Automated Data Processing for Routine Profiling of N-Glycans in Immunoglobulins J. Am. Soc. Mass Spectrom. (2014) 25: 999, herein each incorporated by reference for all purposes. In some embodiments, amount can be determined or calculated as mole percent incorporation.

### Modulating Amounts of Glycans

“Modulating”, as used herein, means to change by decreasing or increasing, and accordingly, in exemplary aspects, the methods disclosed herein comprise increasing and/or decreasing the amount of glycans of the antibody. In exemplary aspects, the methods of the present disclosure comprise increasing the glycans (e.g., galactosylated glycans, G1, G1a, G1b and/or G2 galactosylated species, afucosylated glycans, core fucose, high mannose glycans, M5 high mannose species, or a combination thereof) of the antibody, to any degree or level relative to a control or a reference antibody. In exemplary instances, the method comprises increasing the glycans by at least or about 1% to about 100% (e.g., at least or about 1%, at least or about 2%, at least or about 3%, at least or about 4%, at least or about 5%, at least or about 6%, at least or about 7%, at least or about 8%, at least or about 9%, at least or about 9.5%, at least or about 9.8%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 100%) relative to a control or reference antibody. In exemplary embodiments, the method comprises increasing the glycans by 100% or more, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to a control or a reference antibody. In exemplary embodiments, the method comprises increasing the glycans by at least about 1.5-fold, relative to a control or a reference antibody. In exemplary embodiments, the method comprises increasing the glycans by at least about 2-fold, relative to a control or a reference antibody. In exemplary embodiments, the method comprises increasing the glycans by at least about 3-fold, relative to a control or a reference antibody. In exemplary embodiments, the method comprises increasing the glycans by at least about 4-fold or about 5-fold, relative to a control or a reference antibody. In exemplary embodiments, the method comprises increasing the glycans by at least about 6-fold, about 7-fold, about 8-fold, about 9-fold, or about 10-fold, relative to a control or a reference antibody. In exemplary embodiments, the method comprises increasing the glycans by at least about by an amount falling within the range of about 0.5-fold to about 8-fold, relative to a control or a reference antibody.

In exemplary aspects, the methods of the present disclosure comprise decreasing the glycans (e.g., galactosylated glycans, G1, G1a, G1b and/or G2 galactosylated species, afucosylated glycans, core fucose, high mannose glycans, M5 high mannose species, or a combination thereof) of the antibody, to any degree or level relative to a control or a reference antibody. In exemplary instances, the method comprises decreasing the glycans by at least or about 1% to about 100% (e.g., at least or about 1%, at least or about 2%, at least or about 3%, at least or about 4%, at least or about 5%, at least or about 6%, at least or about 7%, at least or about 8%, at least or about 9%, at least or about 9.5%, at least or about 9.8%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 100%) relative to a control or a reference antibody. In exemplary embodiments, the method comprises decreasing the glycans by 100% or more, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to a control or a reference antibody. In exemplary embodiments, the method comprises decreasing the glycans by at least about 1.5-fold, relative to a control or a reference antibody. In exemplary embodiments, the method comprises decreasing the glycans by at least about 2-fold, relative to a control or a reference antibody. In exemplary embodiments, the method comprises decreasing the glycans by at least about 3-fold, relative to a control or a reference antibody. In exemplary embodiments, the method comprises decreasing the glycans by at least about 4-fold or about 5-fold, relative to a control or a reference antibody. In exemplary embodiments, the method comprises decreasing the glycans by at least about 6-fold, about 7-fold, about 8-fold, about 9-fold, or about 10-fold, relative to a control or a reference antibody. In exemplary embodiments, the method comprises decreasing the glycans by at least about by an amount falling within the range of about 0.5-fold to about 8-fold, relative to a control or a reference antibody.

In exemplary aspects, the methods of the present disclosure comprise modulating (i.e. increasing or decreasing) the amount of the glycans (e.g., galactosylated glycans, G1, G1a, G1b and/or G2 galactosylated species, afucosylated glycans, core fucose, high mannose glycans, M5 high mannose species, or a combination thereof) of the antibody to a total amount of at least or about 0.5%, at least or about 1%, at least or about 2%, at least or about 3%, at least or about 5%, at least or about 7%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97% or at least or about 98% or increased or decreased to a total amount in the range of at least or about 0.5% to 98%.

In exemplary embodiments of the methods of the present disclosure, the method comprises modulating the amount of galactosylated glycans, including, e.g., terminal (3-galactose or G1, G1a, G1b and/or G2 galactosylated species, of the antibody to modulate ADCP activity of the antibody. In exemplary aspects, the method comprises increasing the amount of galactosylated glycans, including, e.g., terminal β-galactose or G1, G1a, G1b and/or G2 galactosylated species, of the antibody to increase ADCP activity of the antibody. In exemplary instances, the method comprises increasing the amount of galactosylated glycans including, e.g., the amount of terminal β-galactose or amount of G1, G1a, G1b and/or G2 galactosylated species of trastuzumab by about 1 percent to increase the ADCP activity of the trastuzumab antibody by about 2.5, about 2.8, about 2.88 or about 3 percent. In some instances, the method comprises increasing the amount of galactosylated glycans including, e.g., the amount of terminal β-galactose or amount of G1, G1a, G1b and/or G2 galactosylated species of trastuzumab by about 1 percent to increase the ADCP activity of the trastuzumab antibody by about 2.88 percent.

In exemplary aspects, the method comprises decreasing the amount of galactosylated glycans including, e.g., terminal β-galactose or G1, G1a, G1b and/or G2 galactosylated species, of the antibody to decrease ADCP activity of the antibody. In exemplary instances, the method comprises decreasing the amount of galactosylated glycans including, e.g., the amount of terminal β-galactose or amount of G1, G1a, G1b and/or G2 galactosylated species of trastuzumab by about 1 percent to decrease the ADCP activity of the trastuzumab antibody by about 2.5, about 2.8, about 2.88 or about 3 percent. In some instances, the method comprises decreasing the amount of galactosylated glycans including, e.g., the amount of terminal β-galactose or amount of G1, G1a, G1b and/or G2 galactosylated species of trastuzumab by about 1 percent to decrease the ADCP activity of the trastuzumab antibody by about 2.88 percent.

In exemplary embodiments of the methods of the present disclosure, the method comprises modulating the amount of afucosylated glycans or the amount of core fucose of the antibody to modulate ADCP activity of the antibody. In exemplary aspects, the method comprises increasing ADCP activity of an antibody by decreasing the amount of afucosylated glycans or increasing the amount of core fucose of the antibody. In exemplary aspects, the method comprises decreasing the amount of afucosylated glycans of a rituximab antibody or increasing the amount of core fucose of a rituximab antibody by about 1 percent to increase ADCP activity of the rituximab antibody by about 0.5, about 0.7, about 0.75, about 0.8 or about 1 percent. In some aspects, the method comprises decreasing the amount of afucosylated glycans of a rituximab antibody or increasing the amount of core fucose of a rituximab antibody by about 1 percent to increase ADCP activity of the rituximab antibody by about 0.75 percent.

In other embodiments of the methods of the present disclosure, the method comprises increasing ADCP activity of an antibody by increasing the amount of afucosylated glycans or decreasing the amount of core fucose of the antibody. In exemplary aspects, the method comprises increasing the amount of afucosylated glycans of a trastuzumab antibody or decreasing the amount of core fucose of a trastuzumab antibody by about 1 percent to increase ADCP activity of the trastuzumab antibody by about 0.5, about 0.56, about 0.6 or about 1 percent. In some aspects, the method comprises increasing the amount of afucosylated glycans of a trastuzumab antibody or decreasing the amount of core fucose of a trastuzumab antibody by about 1 percent to increase ADCP activity of the trastuzumab antibody by about 0.56 percent.

In other exemplary aspects, the method comprises decreasing ADCP activity of an antibody by decreasing the amount of afucosylated glycans or increasing the amount of core fucose of the antibody. In exemplary aspects, the method comprises decreasing the amount of afucosylated glycans of a trastuzumab antibody or increasing the amount of core fucose of a trastuzumab antibody by about 1 percent to decrease ADCP activity of the trastuzumab antibody by about 0.5, about 0.56, about 0.6 or about 1 percent. In some aspects, the method comprises decreasing the amount of afucosylated glycans of a trastuzumab antibody or increasing the amount of core fucose of a trastuzumab antibody by about 1 percent to decrease ADCP activity of the trastuzumab antibody by about 0.56 percent.

In other embodiments of the methods of the present disclosure, the method comprises decreasing ADCP activity of an antibody by increasing the amount of afucosylated glycans or decreasing the amount of core fucose of the antibody. In exemplary aspects, the method comprises increasing the amount of afucosylated glycans of a rituximab antibody or decreasing the amount of core fucose of a rituximab antibody by about 1 percent to decrease ADCP activity of the rituximab antibody by about 0.5, about 0.7, about 0.75, about 0.8 or about 1 percent. In some aspects, the method comprises increasing the amount of afucosylated glycans of a rituximab antibody or decreasing the amount of core fucose of a rituximab antibody by about 1 percent to decrease ADCP activity of the rituximab antibody by about 0.75 percent.

In exemplary instances, the afucosylated glycans modulated (increased or decreased) on the trastuzumab or rituximab antibody include one or more of the afucosylated glycans selected from the group consisting of: A1G0, A1G1, A2G0, A2G1a, A2G1b, A2G2, and A1G1M5.

In exemplary embodiments of the methods of the present disclosure, the method comprises modulating the amount of high mannose glycans, including, e.g., M5 high mannose species, of the antibody to modulate ADCP activity of the antibody, e.g., an IgG1 antibody. In some instances, the IgG1 antibody is an anti-HER2 antibody or an anti-CD20 antibody. In exemplary aspects, the method comprises increasing the amount of high mannose glycans of an anti-HER2 antibody to decrease the ADCP activity of the antibody composition; or decreasing the amount of high mannose glycans of an anti-HER2 antibody to increase the ADCP activity of the antibody composition. In exemplary instances, the method comprises increasing the amount of high mannose glycans of the anti-CD20 antibody to decrease the ADCP activity of the antibody composition; or decreasing the amount of high mannose glycans of the anti-CD20 antibody to increase the ADCP activity of the antibody composition. In exemplary aspects, the high mannose glycans increased or decreased to modulate ADCP activity in the IgG1 antibody (such as trastuzumab or rituximab) includes one or more of the high mannose species selected from the group consisting of: HM5, HM6, HM7, HM8, and HM9.

In exemplary aspects, the method comprises increasing the amount of high mannose glycans, including, e.g., M5 high mannose species, of the antibody to decrease ADCP activity of the antibody. In exemplary instances, the method comprises increasing the amount of high mannose glycans, including, e.g., M5 high mannose species, of the IgG1 antibody (including, e.g., trastuzumab or rituximab) by about 1 percent to decrease the ADCP activity of the IgG1 antibody (including, e.g., trastuzumab or rituximab) by about 1%, about 1.2%, about 1.31%, about 1.5%, about 1.7%, about 2%, about 2.11%, about 2.5%, or in the range of about 1% to about 2.5%. In some instances, the method comprises increasing the amount of high mannose glycans, including, e.g., M5 high mannose species, of the IgG1 antibody (including, e.g., trastuzumab or rituximab) by about 1 percent to decrease the ADCP activity of the IgG1 antibody (including, e.g., trastuzumab or rituximab) by about 1.31% or about 2.11%.

In exemplary aspects, the method comprises decreasing the amount of high mannose glycans, including, e.g., M5 high mannose species, of the antibody to increase ADCP activity of the antibody. In exemplary instances, the method comprises decreasing the amount of high mannose glycans, including, e.g., M5 high mannose species, of the IgG1 antibody (including, e.g., trastuzumab or rituximab) by about 1 percent to increase the ADCP activity of the IgG1 antibody (including, e.g., trastuzumab or rituximab) by about 1%, about 1.2%, about 1.31%, about 1.5%, about 1.7%, about 2%, about 2.11%, about 2.5%. or in the range of about 1% to about 2.5%. In some instances, the method comprises decreasing the amount of high mannose glycans, including, e.g., M5 high mannose species, of the IgG1 antibody (including, e.g., trastuzumab or rituximab) by about 1 percent to increase the ADCP activity of the IgG1 antibody (including, e.g., trastuzumab or rituximab) by about 1.31% or about 2.11%.

In exemplary embodiments of the methods of the present disclosure, the method comprises modulating the amount of a combination of galactosylated glycans (including, e.g., terminal β-galactose or G1, G1a, G1b and/or G2 galactosylated species), afucosylated glycans or core fucose, and/or high mannose glycans (including, e.g., M5 high mannose species) to modulate ADCP activity of an IgG1 antibody, including, e.g., trastuzumab or rituximab. In exemplary embodiments of the methods of the present disclosure, the method comprises modulating ADCP activity of an IgG1 antibody (including, e.g., trastuzumab or rituximab) by increasing or decreasing: (1) the amount of terminal β-galactose in the antibody and/or the amount of G1, G1a, G1b and/or G2 galactosylated species of the antibody; (2) the amount of core fucose in the antibody and/or the amount of afucosylated species of the antibody; and/or (3) the amount of high mannose in the antibody or the amount of M5 high mannose species of the antibody.

In some embodiments of the methods of the present disclosure, the method comprises increasing ADCP activity of an IgG1 antibody (including, e.g., trastuzumab or rituximab) by: (1) increasing the amount of terminal β-galactose in the antibody or increasing the amount of G1, G1a, G1b and/or G2 galactosylated species of the antibody; (2) decreasing the amount of core fucose in the antibody or increasing the amount of afucosylated species of the antibody; and/or (3) decreasing the amount high mannose in the antibody or decreasing the amount of M5 high mannose species of the antibody.

In some other embodiments of the methods of the present disclosure, the method comprises decreasing ADCP activity of an IgG1 antibody (including, e.g., trastuzumab or rituximab) comprising: (1) decreasing the amount of terminal β-galactose in the antibody or decreasing the amount of G1, G1a, G1b and/or G2 galactosylated species of the antibody; (2) increasing the amount of core fucose in the antibody or decreasing the amount of afucosylated species of the antibody; and/or (3) increasing the amount high mannose in the antibody or increasing the amount of M5 high mannose species of the antibody.

### Methods of Engineering ADCP Activity of an Antibody

The methods provided herein also include methods of matching the ADCP activity of a first, reference antibody and the ADCP activity of a second antibody by modulating the amount of glycans (e.g., galactosylated glycans, terminal β-galactose, G1, G1a, G1b and/or G2 galactosylated species, afucosylated glycans, core fucose, high mannose glycans, M5 high mannose species, or a combination thereof) in the second antibody to match the ADCP activity of the first, reference antibody. In exemplary instances, the method comprises changing the ADCP activity of a second antibody to match the ADCP activity of a first, reference antibody by modulating the amount of glycans (e.g., galactosylated glycans, terminal β-galactose, G1, G1a, G1b and/or G2 galactosylated species, afucosylated glycans, core fucose, high mannose glycans, M5 high mannose species, or a combination thereof) in the second antibody to match the ADCP activity of the first, reference antibody. In exemplary aspects, the methods comprise determining or measuring the ADCP activity of the second antibody and/or the reference antibody using the methods described herein. In exemplary aspects, determining or measuring the ADCP activity of a reference or second antibody occurs: (i) before modulating the amount of glycans in the antibody, (ii) after modulating the amount of glycans in the antibody; or (iii) before and after modulating the amount of glycans in the antibody.

For example, in some exemplary embodiments of the methods of the present disclosure, the method comprises matching the ADCP of a reference antibody by (1) determining the ADCP activity of a reference antibody or a reference IgG1 antibody; (2) determining the ADCP activity of a second antibody having the same antibody sequence as the reference antibody; and (3) changing the ADCP activity of the second antibody by increasing or decreasing the amount of glycans (e.g., galactosylated glycans, terminal β-galactose, G1, G1a, G1b and/or G2 galactosylated species, afucosylated glycans, core fucose, high mannose glycans, M5 high mannose species, or a combination thereof) of the second antibody, such that the ADCP activity of the second antibody after increasing or decreasing the amount of glycans is the same as the reference antibody or within about 10%, about 15%, about 20%, about 25%, about 30% or about 35% of the reference antibody or within the range of about 1% to about 35% of the reference antibody. In some instances, step 1 (“determining the ADCP activity of a reference antibody or a reference IgG1 antibody”) occurs before, after or at the same time as step 2 (“determining the ADCP activity of a second antibody . . . ”) and/or step 3 (“changing the ADCP activity of the second antibody . . . ”); while in other instances, step 2 occurs before, after or at the same time as step 1 and/or step 3.

In some embodiments, the method comprises matching the ADCP activity of a reference trastuzumab antibody by: (1) determining the ADCP activity of a reference trastuzumab antibody; (2) determining the ADCP activity of a second antibody having the same antibody sequence as the reference trastuzumab antibody; and (3) increasing the ADCP activity of the second antibody by increasing the amount of terminal β-galactose in the second antibody or increasing the amount of G1, G1a, G1b and/or G2 galactosylated species of the second antibody, wherein the ADCP activity of the second antibody after increasing terminal β-galactose or G1, G1a, G1b and/or G2 galactosylated species in the second antibody is the same as the reference antibody or within about 10%, about 15%, about 20%, about 25%, about 30% or about 35% of the reference antibody or within the range of about 1% to about 35% of the reference antibody. In some instances, step 1 (“determining the ADCP activity of a reference trastuzumab antibody”) occurs before, after or at the same time as step 2 (“determining the ADCP activity of a second antibody . . . ”) and/or step 3 (“increasing the ADCP activity of the second antibody . . . ”); while in other instances, step 2 occurs before, after or at the same time as step 1 and/or step 3.

In some other embodiments, the method comprises matching the ADCP activity of a reference trastuzumab antibody by: (1) determining the ADCP activity of a reference trastuzumab antibody; (2) determining the ADCP activity of a second antibody having the same antibody sequence as the reference trastuzumab antibody; and (3) decreasing the ADCP activity of the second antibody by decreasing the amount of terminal β-galactose in the second antibody or decreasing the amount of G1, G1a, G1b and/or G2 galactosylated species of the second antibody, wherein the ADCP activity of the second antibody after decreasing terminal β-galactose or G1, G1a, G1b and/or G2 galactosylated species in the second antibody is the same as the reference antibody or within about 10%, about 15%, about 20%, about 25%, about 30% or about 35% of the reference antibody or within the range of about 1% to about 35% of the reference antibody. In some instances, step 1 (“determining the ADCP activity of a reference trastuzumab antibody”) occurs before, after or at the same time as step 2 (“determining the ADCP activity of a second antibody . . . ”) and/or step 3 (“decreasing the ADCP activity of the second antibody . . . ”); while in other instances, step 2 occurs before, after or at the same time as step 1 and/or step 3.

In some embodiments, the method comprises matching the ADCP activity of a reference trastuzumab antibody by: (1) determining the ADCP activity of a reference trastuzumab antibody; (2) determining the ADCP activity of a second antibody having the same antibody sequence as the reference trastuzumab antibody; and (3) increasing the ADCP activity of the second antibody by decreasing the amount of core fucose in the second antibody or increasing the amount of afucosylated species of the second antibody, wherein the ADCP activity of the second antibody after decreasing core fucose or increasing afucosylated species is the same as the reference antibody or within about 10%, about 15%, about 20%, about 25%, about 30% or about 35% of the reference antibody or within the range of about 1% to about 35% of the reference antibody. In some instances, step 1 (“determining the ADCP activity of a reference trastuzumab antibody”) occurs before, after or at the same time as step 2 (“determining the ADCP activity of a second antibody . . . ”) and/or step 3 (“increasing the ADCP activity of the second antibody . . . ”); while in other instances, step 2 occurs before, after or at the same time as step 1 and/or step 3. In some embodiments, the method comprises matching the ADCP activity of a reference trastuzumab antibody by: (1) determining the ADCP activity of a reference trastuzumab antibody; (2) determining the ADCP activity of a second antibody having the same antibody sequence as the reference trastuzumab antibody; and (3) decreasing the ADCP activity of the second antibody by increasing the amount of core fucose in the second antibody or decreasing the amount of afucosylated species of the second antibody, wherein the ADCP activity of the second antibody after increasing core fucose or decreasing afucosylated species is the same as the reference antibody or within about 10%, about 15%, about 20%, about 25%, about 30% or about 35% of the reference antibody or within the range of about 1% to about 35% of the reference antibody. In some instances, step 1 (“determining the ADCP activity of a reference trastuzumab antibody”) occurs before, after or at the same time as step 2 (“determining the ADCP activity of a second antibody . . . ”) and/or step 3 (“decreasing the ADCP activity of the second antibody . . . ”); while in other instances, step 2 occurs before, after or at the same time as step 1 and/or step 3.

In some embodiments, the method comprises matching the ADCP activity of a reference rituximab antibody by: (1) determining the ADCP activity of a reference rituximab antibody; (2) determining the ADCP activity of a second antibody having the same antibody sequence as the reference rituximab antibody; and (3) increasing the ADCP activity of the second antibody by increasing the amount of core fucose in the second antibody or decreasing the amount of afucosylated species of the second antibody, wherein the ADCP activity of the second antibody after increasing core fucose or decreasing afucosylated species is the same as the reference antibody or within about 10%, about 15%, about 20%, about 25%, about 30% or about 35% of the reference antibody or within the range of about 1% to about 35% of the reference antibody. In some instances, step 1 (“determining the ADCP activity of a reference rituximab antibody”) occurs before, after or at the same time as step 2 (“determining the ADCP activity of a second antibody . . . ”) and/or step 3 (“increasing the ADCP activity of the second antibody . . . ”); while in other instances, step 2 occurs before, after or at the same time as step 1 and/or step 3.

In some embodiments, the method comprises matching the ADCP activity of a reference rituximab antibody by: (1) determining the ADCP activity of a reference rituximab antibody; (2) determining the ADCP activity of a second antibody having the same antibody sequence as the reference rituximab antibody; and (3) decreasing the ADCP activity of the second antibody by decreasing the amount of core fucose in the second antibody or increasing the amount of afucosylated species of the second antibody, wherein the ADCP activity of the second antibody after decreasing core fucose or increasing afucosylated species is the same as the reference antibody or within about 10%, about 15%, about 20%, about 25%, about 30% or about 35% of the reference antibody or within the range of about 1% to about 35% of the reference antibody. In some instances, step 1 (“determining the ADCP activity of a reference rituximab antibody”) occurs before, after or at the same time as step 2 (“determining the ADCP activity of a second antibody . . . ”) and/or step 3 (“decreasing the ADCP activity of the second antibody . . . ”); while in other instances, step 2 occurs before, after or at the same time as step 1 and/or step 3.

In some embodiments, the method comprises matching the ADCP activity of a reference IgG1 antibody (including, e.g., trastuzumab or rituximab) by: (1) determining the ADCP activity of a reference IgG1 antibody (including, e.g., trastuzumab or rituximab); (2) determining the ADCP activity of a second antibody having the same antibody sequence as the reference antibody; and (3) increasing the ADCP activity of the second antibody by decreasing the amount of high mannose in the second antibody or decreasing the amount of M5 high mannose species of the second antibody, wherein the ADCP activity of the second antibody after decreasing high mannose or M5 high mannose species is the same as the reference antibody or within about 10%, about 15%, about 20%, about 25%, about 30% or about 35% of the reference antibody or within the range of about 1% to about 35% of the reference antibody. In some instances, step 1 (“determining the ADCP activity of a reference IgG1 antibody”) occurs before, after or at the same time as step 2 (“determining the ADCP activity of a second antibody . . . ”) and/or step 3 (“increasing the ADCP activity of the second antibody . . . ”); while in other instances, step 2 occurs before, after or at the same time as step 1 and/or step 3.

In some embodiments, the method comprises matching the ADCP activity of a reference IgG1 antibody (including, e.g., trastuzumab or rituximab) by: (1) determining the ADCP activity of a reference IgG1 antibody (including, e.g., trastuzumab or rituximab); (2) determining the ADCP activity of a second antibody having the same antibody sequence as the reference antibody; and (3) decreasing the ADCP activity of the second antibody by increasing the amount of high mannose in the second antibody or increasing the amount of M5 high mannose species of the second antibody, wherein the ADCP activity of the second antibody after increasing high mannose or M5 high mannose species is the same as the reference antibody or within about 10%, about 15%, about 20%, about 25%, about 30% or about 35% of the reference antibody or within the range of about 1% to about 35% of the reference antibody. In some instances, step 1 (“determining the ADCP activity of a reference IgG1 antibody”) occurs before, after or at the same time as step 2 (“determining the ADCP activity of a second antibody . . . ”) and/or step 3 (“decreasing the ADCP activity of the second antibody . . . ”); while in other instances, step 2 occurs before, after or at the same time as step 1 and/or step 3.

In addition to methods of matching the ADCP of a reference antibody, the methods provided herein also include methods of engineering an antibody with a specific ADCP activity by modulating the amount of glycans (e.g., galactosylated glycans, terminal β-galactose, G1, G1a, G1b and/or G2 galactosylated species, afucosylated glycans, core fucose, high mannose glycans, M5 high mannose species, or a combination thereof) of the antibody to achieve a target ADCP activity.

For example, in some exemplary embodiments of the methods of the present disclosure, the method comprises engineering a specific target ADCP activity in an antibody (including, e.g., trastuzumab or rituximab) by: (1) determining the ADCP activity of an antibody or an IgG1 antibody; (2) determining or specifying a target ADCP activity; and (3) changing the ADCP activity of the antibody by increasing or decreasing the amount of glycans (e.g., galactosylated glycans, terminal β-galactose, G1, G1a, G1b and/or G2 galactosylated species, afucosylated glycans, core fucose, high mannose glycans, M5 high mannose species, or a combination thereof) of the antibody, such that the ADCP activity of the antibody after increasing or decreasing the amount of glycans is the same as the target ADCP activity or within about 10%, about 15%, about 20%, about 25%, about 30% or about 35% of the target ADCP activity or within the range of about 1% to about 35% of the target ADCP activity. In some instances, step 1 (“determining the ADCP activity of an antibody or an IgG1 antibody”) occurs before, after or at the same time as step 2 (“determining or specifying a target ADCP activity”) and/or step 3 (“changing the ADCP activity of the antibody . . . ”); while in other instances, step 2 occurs before, after or at the same time as step 1 and/or step 3.

In some embodiments, the method comprises engineering a specific target ADCP activity of a trastuzumab antibody by: (1) determining the ADCP activity of a trastuzumab antibody; (2) determining or specifying a target ADCP activity; and (3) increasing the ADCP activity of the trastuzumab antibody by increasing the amount of terminal β-galactose in the antibody or increasing the amount of G1, G1a, G1b and/or G2 galactosylated species of the antibody, wherein the ADCP activity of the trastuzumab antibody after increasing terminal β-galactose or G1, G1a, G1b and/or G2 galactosylated species in the antibody is the same as the target ADCP activity or within about 10%, about 15%, about 20%, about 25%, about 30% or about 35% of the target ADCP activity or within the range of about 1% to about 35% of the target ADCP activity. In some instances, step 1 (“determining the ADCP activity of a trastuzumab antibody”) occurs before, after or at the same time as step 2 (“determining or specifying a target ADCP activity”) and/or step 3 (“increasing the ADCP activity of the trastuzumab antibody . . . ”); while in other instances, step 2 occurs before, after or at the same time as step 1 and/or step 3.

In some other embodiments, the method comprises engineering a specific target ADCP activity of a trastuzumab antibody by: (1) determining the ADCP activity of a trastuzumab antibody; (2) determining or specifying a target ADCP activity; and (3) decreasing the ADCP activity of the trastuzumab antibody by decreasing the amount of terminal β-galactose in the antibody or decreasing the amount of G1, G1a, G1b and/or G2 galactosylated species of the antibody, wherein the ADCP activity of the trastuzumab antibody after decreasing terminal β-galactose or G1, G1a, G1b and/or G2 galactosylated species in the antibody is the same as the target ADCP activity or within about 10%, about 15%, about 20%, about 25%, about 30% or about 35% of the target ADCP activity or within the range of about 1% to about 35% of the target ADCP activity. In some instances, step 1 (“determining the ADCP activity of a trastuzumab antibody”) occurs before, after or at the same time as step 2 (“determining or specifying a target ADCP activity”) and/or step 3 (“decreasing the ADCP activity of the trastuzumab antibody . . . ”); while in other instances, step 2 occurs before, after or at the same time as step 1 and/or step 3.

In some embodiments, the method comprises engineering a specific target ADCP activity of a trastuzumab antibody by: (1) determining the ADCP activity of a trastuzumab antibody; (2) determining or specifying a target ADCP activity; and (3) increasing the ADCP activity of the trastuzumab antibody by decreasing the amount of core fucose in the antibody or increasing the amount of afucosylated species of the antibody, wherein the ADCP activity of the trastuzumab antibody after decreasing core fucose or increasing afucosylated species is the same as the target ADCP activity or within about 10%, about 15%, about 20%, about 25%, about 30% or about 35% of the target ADCP activity or within the range of about 1% to about 35% of the target ADCP activity. In some instances, step 1 (“determining the ADCP activity of a trastuzumab antibody”) occurs before, after or at the same time as step 2 (“determining or specifying a target ADCP activity”) and/or step 3 (“increasing the ADCP activity of the trastuzumab antibody . . . ”); while in other instances, step 2 occurs before, after or at the same time as step 1 and/or step 3.

In some embodiments, the method comprises engineering a specific target ADCP activity of a trastuzumab antibody by: (1) determining the ADCP activity of a trastuzumab antibody; (2) determining or specifying a target ADCP activity; and (3) decreasing the ADCP activity of the trastuzumab antibody by increasing the amount of core fucose in the antibody or decreasing the amount of afucosylated species of the antibody, wherein the ADCP activity of the trastuzumab antibody after increasing core fucose or decreasing afucosylated species is the same as the target ADCP activity or within about 10%, about 15%, about 20%, about 25%, about 30% or about 35% of the target ADCP activity or within the range of about 1% to about 35% of the target ADCP activity. In some instances, step 1 (“determining the ADCP activity of a trastuzumab antibody”) occurs before, after or at the same time as step 2 (“determining or specifying a target ADCP activity”) and/or step 3 (“decreasing the ADCP activity of the trastuzumab antibody . . . ”); while in other instances, step 2 occurs before, after or at the same time as step 1 and/or step 3.

In some embodiments, the method comprises engineering a specific target ADCP activity of a rituximab antibody by: (1) determining the ADCP activity of a rituximab antibody; (2) determining or specifying a target ADCP activity; and (3) increasing the ADCP activity of the rituximab antibody by increasing the amount of core fucose in the antibody or decreasing the amount of afucosylated species of the antibody, wherein the ADCP activity of the rituximab antibody after increasing core fucose or decreasing afucosylated species is the same as the target ADCP activity or within about 10%, about 15%, about 20%, about 25%, about 30% or about 35% of the target ADCP activity or within the range of about 1% to about 35% of the target ADCP activity. In some instances, step 1 (“determining the ADCP activity of a rituximab antibody”) occurs before, after or at the same time as step 2 (“determining or specifying a target ADCP activity”) and/or step 3 (“increasing the ADCP activity of the rituximab antibody . . . ”); while in other instances, step 2 occurs before, after or at the same time as step 1 and/or step 3.

In some embodiments, the method comprises engineering a specific target ADCP activity of a rituximab antibody by: (1) determining the ADCP activity of a rituximab antibody; (2) determining or specifying a target ADCP activity; and (3) decreasing the ADCP activity of the rituximab antibody by decreasing the amount of core fucose in the antibody or increasing the amount of afucosylated species of the antibody, wherein the ADCP activity of the rituximab antibody after decreasing core fucose or increasing afucosylated species is the same as the target ADCP activity or within about 10%, about 15%, about 20%, about 25%, about 30% or about 35% of the target ADCP activity or within the range of about 1% to about 35% of the target ADCP activity. In some instances, step 1 (“determining the ADCP activity of a rituximab antibody”) occurs before, after or at the same time as step 2 (“determining or specifying a target ADCP activity”) and/or step 3 (“decreasing the ADCP activity of the rituximab antibody . . . ”); while in other instances, step 2 occurs before, after or at the same time as step 1 and/or step 3.

In some embodiments, the method comprises engineering a specific target ADCP activity of an IgG1 antibody (including, e.g., trastuzumab or rituximab) by: (1) determining the ADCP activity of an IgG1 antibody (including, e.g., trastuzumab or rituximab); (2) determining or specifying a target ADCP activity; and (3) increasing the ADCP activity of the IgG1 antibody (including, e.g., trastuzumab or rituximab) by decreasing the amount of high mannose in the antibody or decreasing the amount of M5 high mannose species of the antibody, wherein the ADCP activity of the IgG1 antibody (including, e.g., trastuzumab or rituximab) after decreasing high mannose or M5 high mannose species is the same as the target ADCP activity or within about 10%, about 15%, about 20%, about 25%, about 30% or about 35% of the target ADCP activity or within the range of about 1% to about 35% of the target ADCP activity. In some instances, step 1 (“determining the ADCP activity of an IgG1 antibody”) occurs before, after or at the same time as step 2 (“determining or specifying a target ADCP activity”) and/or step 3 (“increasing the ADCP activity of the IgG1 antibody . . . ”); while in other instances, step 2 occurs before, after or at the same time as step 1 and/or step 3.

In some embodiments, the method comprises engineering a specific target ADCP activity of an IgG1 antibody (including, e.g., trastuzumab or rituximab) by: (1) determining the ADCP activity of a reference IgG1 antibody (including, e.g., trastuzumab or rituximab); (2) determining or specifying a target ADCP activity; and (3) decreasing the ADCP activity of the IgG1 antibody (including, e.g., trastuzumab or rituximab) by increasing the amount of high mannose in the antibody or increasing the amount of M5 high mannose species of the antibody, wherein the ADCP activity of the IgG1 antibody (including, e.g., trastuzumab or rituximab) after increasing high mannose or M5 high mannose species is the same as the target ADCP activity or within about 10%, about 15%, about 20%, about 25%, about 30% or about 35% of the target ADCP activity or within the range of about 1% to about 35% of the target ADCP activity. In some instances, step 1 (“determining the ADCP activity of an IgG1 antibody”) occurs before, after or at the same time as step 2 (“determining or specifying a target ADCP activity”) and/or step 3 (“decreasing the ADCP activity of the IgG1 antibody . . . ”); while in other instances, step 2 occurs before, after or at the same time as step 1 and/or step 3.

### Methods of Modulating Glycans

Suitable methods of modulating the amount of glycans (such as galactosylated glycans (including, e.g., terminal β-galactose or G1, G1a, G1b and/or G2 galactosylated species), afucosylated glycans or glycans containing core fucose, and/or high mannose glycans (including, e.g., M5 high mannose species) on glycoproteins, including antibodies, are known in the art. See, e.g., Zhang et al., Drug Discovery Today 21(5): 2016). Thus, in some aspects, glycosylation-competent cells—which can be used to recombinantly produce a glycoprotein, including antibodies—are cultured under particular conditions to achieve the desired level of glycans.

For example, International Patent Publication Nos. WO 2013/114164; WO 2013/114245; WO 2013/114167; WO 2015128793; and WO 2016/089919 each teach recombinant cell culturing techniques useful to modulate glycans, such as galactosylated glycans (including, e.g., terminal β-galactose or G1, G1a, G1b and/or G2 galactosylated species), afucosylated glycans or glycans containing core fucose, and/or high mannose glycans (including, e.g., M5 high mannose species) including: methods of obtaining glycoproteins having increased percentage of total afucosylated glycans (WO 2013/114164); methods of obtaining glycoproteins having increased percentage of Man5 glycans and/or afucosylated glycans (WO 2013/114245); methods of obtaining glycoproteins having specific amounts of high mannose glycans, afucosylated glycans and G0F glycans (WO 2013/114167); methods of obtaining glycoproteins having high mannose glycan and reduced galactosylation and/or high galactosylated glycans (WO 2015128793); and methods of manipulating the fucosylated glycan content on a recombinant protein (WO 2016/089919). The cell culture methods described by WO 2013/114164; WO 2013/114245; WO 2013/114167; WO 2015128793; and WO 2016/089919 include modifying one or more cell culture parameters such as temperature, pH, culturing cells with manganese ion or salts thereof (e.g., 0.35 μM to about 20 μM Manganese) and/or culturing cells with copper (e.g., 10 to 100) and manganese (e.g., 50 to 1000 nM) to modulate specific glycans.

Additionally, International Patent Publication No. WO 2015/140700 describes culturing cells in the presence of betaine to increase afucosylated glycans, or culturing cells with manganese, galactose and betaine to obtain target values of mannosylated, galactosylated and afucosylated glycans. U.S. Patent Application Publication No. 2014/0356910 teaches methods of increasing high mannose glycoforms by manipulating the mannose to total hexose ratio in the cell culture media formulation. Pacis et al., Biotechnology and Bioengineering 108(10): 2348-2358 (2011) teaches obtaining high levels of Man5 glycans by increasing cell culture medium osmolality levels and extending culture duration. Similarly, Konno et al., Cytotechnology 64: 249-3+6 (2012) describes methods of controlling antibody fucose content through culture medium osmolality. Wong et al., Biotechnology and Bioengineering 89(2): 164-177 (2004) teaches methods of decreasing recombinant protein sialylation and increasing high mannose glycans by using low glutamine fed-batch cultures. International Patent Publication No. WO 2017/079165 describes methods of increasing or decreasing afucosylated or fucosylated forms of recombinant proteins by using host cells genetically modified to have no GMD or FX and culturing the host cell with fucose. International Patent Publication No. WO 2017/134667 describes culturing cells with nicotinamide and fucose to produce antibodies having decreased levels of afucosylation. Sha et al., TIBs 34(10): 835-846 (2016) also reviews several methods of modulating glycans, including, for example, culturing with uridine, manganese, and galactose to increase galactosylation levels on antibodies, and using mannose as a carbon source to increase high mannose glycoforms.

Accordingly, the methods of the present disclosure, in exemplary aspects, comprises adopting one or more of the practices, cell culture media and/or cell culture conditions taught in any one or more of the above references or other reference described herein, in order to modulate the amounts of the galactosylated glycans (including, e.g., terminal β-galactose or G1, G1a, G1b and/or G2 galactosylated species), afucosylated glycans or glycans containing core fucose, and/or high mannose glycans (including, e.g., M5 high mannose species). In exemplary aspects, the method comprises culturing glycosylation-competent cells expressing the antibody in a cell culture medium under conditions which modulate the level(s) of the galactosylated glycans (including, e.g., terminal β-galactose or G1, G1a, G1b and/or G2 galactosylated species), afucosylated glycans or glycans containing core fucose, and/or high mannose glycans (including, e.g., M5 high mannose species). For example, the method, in some aspects, comprises culturing glycosylation-competent cells expressing the antibody in a cell culture medium under conditions which modulate the level(s) of the glycan(s), wherein the cell culture medium comprises fucose or fucose and glucose.

In the methods comprising maintaining or culturing cells in cell culture, the cell culture may be maintained according to any set of conditions suitable for a recombinant glycosylated protein or antibody production. For example, in some aspects, the cell culture is maintained at a particular pH, temperature, cell density, culture volume, dissolved oxygen level, pressure, osmolality, and the like. In exemplary aspects, the cell culture prior to inoculation is shaken (e.g., at 70 rpm) at 5% CO₂ under standard humidified conditions in a CO₂ incubator. In exemplary aspects, the method comprises culturing glycosylation-competent cells expressing the antibody in a cell culture medium under conditions which modulate the level(s) of the glycan(s), wherein the osmolality of the cell culture medium is increased to decrease the level of afucosylated glycans of the antibody, e.g., as taught by Konno et al., supra. In exemplary aspects, the method comprises culturing glycosylation-competent cells expressing the antibody in a cell culture medium under conditions which modulate the level(s) of the glycan(s), wherein the pH and the temperature of the cell culture are adjusted, e.g., as taught by WO 2013/114164, WO 2013/114245, WO 2013/114167, or WO 2015/128793, each herein incorporated by reference.

In exemplary aspects, the methods of the disclosure comprise maintaining the glycosylation-competent cells in a cell culture medium at a pH, temperature, osmolality, and dissolved oxygen level suitable for recombinant glycosylated protein or antibody production, as well-known in the art. In exemplary aspects, the cell culture is maintained in a medium suitable for cell growth and/or is provided with one or more feeding media according to any suitable feeding schedule as well-known in the art.

In exemplary aspects, the glycosylation-competent cells are eukaryotic cells, including, but not limited to, yeast cells, filamentous fungi cells, protozoa cells, algae cells, insect cells, or mammalian cells. Such host cells are described in the art. See, e.g., Frenzel, et al., Front Immunol 4: 217 (2013). In exemplary aspects, the eukaryotic cells are mammalian cells. In exemplary aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NS0, GS-NS0, Sp2/0), cells engineered to be deficient in dihydrofolatereductase (DHFR) activity (e.g., DUKX-X11, DG44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinomic human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells (Gaillet et al. 2007; Khan, Adv Pharm Bull 3(2): 257-263 (2013)).

Cells that are not glycosylation-competent can also be transformed into glycosylation-competent cells, e.g. by transfecting them with genes encoding relevant enzymes necessary for glycosylation. Exemplary enzymes include but are not limited to oligosaccharyltransferases, glycosidases, glucosidase I, glucosidease II, calnexin/calreticulin, glycosyltransferases, mannosidases, GlcNAc transferases, galactosyltransferases, and sialyltransferases.

In additional or alternative aspects, the glycosylation-competent cells which recombinantly produce the antibody are genetically modified in a way to modulate the glycans (such as the galactosylated glycans (including, e.g., terminal β-galactose or G1, G1a, G1b and/or G2 galactosylated species), afucosylated glycans or glycans containing core fucose, and/or high mannose glycans (including, e.g., M5 high mannose species) of the antibody. In exemplary aspects, the glycosylation-competent cells are genetically modified to alter activity of an enzyme of the de novo pathway or the salvage pathway. Optionally, the glycosylation-competent cells are genetically modified to knock-out a gene encoding GDP-keto-6-deoxymannonse-3,5-epimerase, 4-reductase. In exemplary embodiments, the glycosylation-competent cells are genetically modified to alter the activity of an enzyme of the de novo pathway or the salvage pathway. These two pathways of fucose metabolism are well-known in the art and shown in FIG. 1C. In exemplary embodiments, the glycosylation-competent cells are genetically modified to alter the activity of any one or more of: a fucosyl-transferase (FUT, e.g., FUT1, FUT2, FUT3, FUT4, FUT5, FUT6, FUT7, FUT8, FUT9), a fucose kinase, a GDP-fucose pyrophosphorylase, GDP-D-mannose-4,6-dehydratase (GMD), and GDP-keto-6-deoxymannose-3,5-epimerase, 4-reductase (FX). In exemplary embodiments, the glycosylation-competent cells are genetically modified to knock-out a gene encoding FX. In exemplary embodiments, the glycosylation-competent cells are genetically modified to alter the activity β(1,4)-N-acetylglucosaminyltransferase III (GNTIII) and/or GDP-6-deoxy-D-lyxo-4-hexulose reductase (RMD). In exemplary aspects, the glycosylation-competent cells are genetically modified to overexpress GNTIII and/or RMD. In exemplary embodiments, the glycosylation-competent cells are genetically modified to have altered beta-galactosyltransferase activity. In some embodiments, the glycosylation-competent cells are genetically modified to modulate the expression level of the gene encoding GDP-keto-6-deoxymannonse-3,5-epimerase, 4-reductase, β1-4 galactosyltransferase, and/or β1-4 N-acetylgalactosaminyltransferase.

Several ways are known in the art for reducing or abolishing fucosylation of Fc-containing molecules, e.g., antibodies. These include recombinant expression in certain mammalian cell lines including a FUT8 knockout cell line, variant CHO line Lec13, rat hybridoma cell line YB2/0, a cell line comprising a small interfering RNA specifically against the FUT8 gene, and a cell line coexpressing β-1,4-N-acetylglucosaminyltransferase III and Golgi α-mannosidase II. Alternatively, the Fc-containing molecule may be expressed in a non-mammalian cell such as a plant cell, yeast, or prokaryotic cell, e.g., E. coli.

In exemplary aspects, targeted glycan amounts are achieved through post-production chemical or enzyme treatment of the antibody. In exemplary aspects, the method of the present disclosure comprises treating the antibody with a chemical or enzyme after the antibody is recombinantly produced. In exemplary aspects, the chemical or enzyme is selected from the group consisting of EndoS; Endo-S2; Endo-D; Endo-M; endoLL; α-fucosidase; β-(1-4)-Galactosidase; Endo-H; Endo F1; Endo F2; Endo F3; β-1,4-galactosyltransferase; kifunensine, and PNGase F. In exemplary aspects, the chemical or enzyme is incubated with the antibody at various times to generate antibodies having different amounts of glycans. In some aspects, the antibody is incubated with β-1,4-galactosyltransferase as described in the Examples. In some additional aspects, antibodies having different levels of galactose can be generated by incubating the antibody with β-1,4-galactosyltransferase for a set period of time, including, but not limited to, about 10 minutes, about 20 minutes, about 30 minutes, about 1 hour, about 2 hours, about 4 hours, about 9 hours or for a period of time falling in the range between about 10 minutes and about 9 hours.

### Methods of Measuring Glycans

Various methods are known in the art for assessing glycoforms present in a glycoprotein-containing composition, including antibodies, or for determining, detecting or measuring a glycoform profile of a particular sample comprising glycoproteins. Suitable methods include, but are not limited to, Hydrophilic Interaction Liquid Chromatography (HILIC), Liquid chromatography-tandem mass spectrometry (LC-MS), positive ion MALDI-TOF analysis, negative ion MALDI-TOF analysis, HPLC, weak anion exchange (WAX) chromatography, normal phase chromatography (NP-HPLC), exoglycosidase digestion, Bio-Gel P-4 chromatography, anion-exchange chromatography and one-dimensional NMR spectroscopy, and combinations thereof. See, e.g., Pace et al., Biotechnol. Prog., 2016, Vol. 32, No. 5 pages 1181-1192; Shah, B. et al. J. Am. Soc. Mass Spectrom. (2014) 25: 999; Mattu et al., JBC 273: 2260-2272 (1998); Field et al., Biochem J 299(Pt 1): 261-275 (1994); Yoo et al., MAbs 2(3): 320-334 (2010) Wuhrer M. et al., Journal of Chromatography B, 2005, Vol. 825, Issue 2, pages 124-133; Ruhaak L. R., Anal Bioanal Chem, 2010, Vol. 397:3457-3481; Kurogochi et al., PLOS One 10(7): e0132848; doi:10.1371/journal.pone.0132848; Thomann et al., PLOS One 10(8): e0134949. Doi:10.1371/journal.pone.0134949; Pace et al., Biotechnol. Prog. 32(5): 1181-1192 (2016); and Geoffrey, R. G. et. al. Analytical Biochemistry 1996, Vol. 240, pages 210-226. Also, the examples set forth herein describe a suitable method for assessing glycoforms present in a glycoprotein containing composition such as an antibody.

### Control

As described herein, some of the methods of the disclosure recite a modulation (e.g., an increase or decrease) effected by such methods that are relative to a “control” or “reference antibody”. In exemplary aspects, with regard to ADCP activity or amount of glycans, the “control” is the level of ADCP activity and/or amount of glycans of the antibody or composition (e.g., a reference antibody) prior to any experimental intervention directed at modulating ADCP activity and/or modulating glycan profile, such as the level of ADCP activity and/or amount of glycans of the antibody or composition (e.g., a reference antibody) when first measured or determined. In certain aspects, a “control” or “reference antibody” can be an antibody that has undergone significant experimental intervention directed at modulating ADCP activity and/or modulating glycan profile but where additional modulation of ADCP activity and/or glycan profile is desired. In these instances, the “control” is the level of ADCP activity and/or amount of glycans of the antibody or composition (e.g., a reference antibody) prior to any additional experimental intervention directed at further modulating ADCP activity and/or further modulating glycan profile.

### Antibody, Fragments, and Protein Products

As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. See, e.g., Janeway et al., “Structure of the Antibody Molecule and the Immunoglobulin Genes”, Immunobiology: The Immune System in Health and Disease, 4th ed. Elsevier Science Ltd./Garland Publishing, (1999).

The term “antibody fragment” or “antibody fragment thereof” refers to a portion of an intact antibody. An “antigen-binding fragment” or “antigen-binding fragment thereof” refers to a portion of an intact antibody that binds to an antigen. An antigen-binding fragment can contain the antigenic determining variable regions of an intact antibody. Examples of antibody fragments antigen-binding fragment include, but are not limited to Fab, Fab′, F(ab′)2, and Fv fragments, linear antibodies, scFvs, and single chain antibodies.

The term “IgG” as used herein refers to a polypeptide belonging to the class of antibodies that are substantially encoded by a recognized immunoglobulin gamma gene. In humans, this class comprises IgG1, IgG2, IgG3, and IgG4. In mice, this class comprises IgG1, IgG2a, IgG2b, and IgG3. The sequences of the heavy chains of human IgG1, IgG2, IgG3 and IgG4 can be found in many sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG1_HUMAN), respectively. In preferred embodiments, the methods and antibodies disclosed herein relate to IgG1 antibodies. In some other preferred embodiments, the methods and antibodies disclosed herein relate to human IgG1 antibodies.

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody with the antigen and hence contribute to the functional activity of an antibody molecule: they are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit.; Chothia et al., J. MoI. Biol, 1987, 196: 901-917; and MacCallum et al., J. MoI. Biol, 1996, 262: 732). Still another standard for characterizing the antigen binding site is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred. See, e.g., Chothia and Lesk, J. MoI. Biol., 1987, 196: 901; Chothia et al., Nature, 1989, 342: 877; Martin and Thornton, J. MoI. Biol, 1996, 263: 800; Antibodies: A Laboratory Manual, Cold Spring Harbor Laboratory, eds. Harlow et al., 1988, each herein incorporated by reference.

The term “variable” refers to the portions of the antibody or immunoglobulin domains that exhibit variability in their sequence and that are involved in determining the specificity and binding affinity of a particular antibody (i.e., the “variable domain(s)”). The pairing of a variable heavy chain (VH) and a variable light chain (VL) together forms a single antigen-binding site.

Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three-dimensional space to form an antigen-binding surface. The variable domains of naturally occurring heavy and light chains each comprise four FRM regions (FR1, FR2, FR3, and FR4), largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding site (see Kabat et al., loc. cit.).

The terms “Fc domain,” “Fc Region,” and “IgG Fc domain” as used herein refer to the portion of an immunoglobulin, e.g., an IgG molecule, that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor. For example, an Fc domain contains the entire second constant domain CH2 (residues at EU positions 231-340 of human IgG1) and the third constant domain CH3 (residues at EU positions 341-447 of human IgG1).

Fc can refer to this region in isolation, or this region in the context of an antibody, or antibody fragment. Polymorphisms have been observed at a number of positions in Fc domains, including but not limited to EU positions 270, 272, 312, 315, 356, and 358. Thus, a “wild type IgG Fc domain” or “WT IgG Fc domain” refers to any naturally occurring IgG Fc region (i.e., any allele). Myriad Fc mutants, Fc fragments, Fc variants, and Fc derivatives are described, e.g., in U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 7,122,637; 7,183,387; 7,332,581; 7,335,742; 7,371,826; 6,821,505; 6,180,377; 7,317,091; 7,355,008; U.S. Patent publication 2004/0002587; and PCT Publication Nos. WO 99/058572, WO 2011/069164 and WO 2012/006635.

The Fc region generally determines the antibody effector function that will ensue after antigen binding. It can recruit molecules in the innate immune system, such as C1q, as well as cytotoxic and antigen-presenting cells via binding interactions with Fcγ receptors. The IgG Fc region contains two conserved N-glycosylation sites at Asn297, one on each heavy chain (see P. M. Rudd. Glycosylation and the immune system. Science, 291 (2001), pp. 2370-2376). Variations in the structure glycans at ASN297 results in subtle changes in structure that influence the interaction of IgG with the immune system. For example, Fc region glycans can directly influence the affinity of IgGs to Fcγ receptors, either by changing the conformation of the Fc region (see S. Krapp, et al. Structural analysis of human IgG-Fc glycoforms reveals correlation between glycosylation and structural integrity J. Mol. Biol., 325 (2003); 979-98931; Y. Mimura, et al. Role of oligosaccharide residues of IgG1-Fc in Fc RIIb binding J. Biol. Chem., 276 (2001), 45539-45547) or through glycan-glycan interactions (see C. Ferrara, et al. Unique carbohydrate-carbohydrate interactions are required for high affinity binding between Fc(RIII and antibodies lacking core fucose. Proc. Natl. Acad. Sci. U.S.A, 108 (2011), 12669-12674), thus strongly influencing their ability to recruit immune effector cells. See also, Zhang et al. Challenges of glycosylation analysis and control: an integrated approach to producing optimal and consistent therapeutic drugs. Drug Discovery Today, (21) 5 (2016) 740-765.

The term “monoclonal antibody” (mAb) as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site or determinant on the antigen, in contrast to conventional (polyclonal) antibody preparations which typically include different antibodies directed against different determinants (or epitopes). In addition to their specificity, the monoclonal antibodies are advantageous in that they are synthesized by the hybridoma culture, hence uncontaminated by other immunoglobulins. The modifier “monoclonal” indicates the character of the antibody as being obtained from a substantially homogeneous population of antibodies and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore system can be used to increase the efficiency of phage antibodies which bind to an epitope of a target antigen (Schier, Human Antibodies Hybridomas 7 (1996), 97-105; Malmborg, J. Immunol. Methods 183 (1995), 7-13).

Another exemplary method of making monoclonal antibodies includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified antibody constructs include humanized variants of non-human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function(s) (see, e.g., U.S. Pat. No. 5,648,260, Kontermann and Dübel (2010), loc. cit. and Little (2009), loc. cit.).

The monoclonal antibodies described in the present invention include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primitized” antibodies comprising variable domain antigen-binding sequences derived from a non-human primate (e.g., Old World Monkey, Ape etc.) and human constant region sequences. A variety of approaches for making chimeric antibodies have been described. See e.g., Morrison et al., Proc. Natl. Acad. ScL U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

Humanized antibodies may also be produced using transgenic animals such as mice that express human heavy and light chain genes but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 0239400).

The term “human antibody” includes antibodies having antibody regions such as variable and constant regions or domains which correspond substantially to human germline immunoglobulin sequences known in the art, including, for example, those described by Kabat et al. (1991) (loc. cit.). The human antibodies, antibody constructs or binding domains of the invention may include amino acid residues not encoded by human germline immunoglobulin sequences (e.g., mutations introduced by random or site-specific mutagenesis in vitro or by somatic mutation in vivo), for example in the CDRs, and in particular, in CDR3. The human antibodies, antibody constructs or binding domains can have at least one, two, three, four, five, or more positions replaced with an amino acid residue that is not encoded by the human germline immunoglobulin sequence. The definition of human antibodies, antibody constructs and binding domains as used herein also contemplates fully human antibodies, which include only non-artificially and/or genetically altered human sequences of antibodies as those can be derived by using technologies or systems such as the Xenomouse.

Advantageously, the methods described herein relate to antibodies comprising an Fc domain, and in exemplary instances, IgG1 antibodies. In exemplary embodiments, the antibody is an IgG1 antibody which has a particular antibody sequence. The term “antibody sequence” refers to the amino acid sequence of an antibody. The phrase used herein “having the same sequence as the reference antibody” refers to an antibody having an identical amino acid sequence to the amino acid sequence of a reference antibody's complementarity determining region (CDR), variable heavy chain (VH) and/or a variable light chain (VL). In preferred embodiments, an antibody “having the same sequence as a reference antibody” as used herein refers to an antibody having the same CDR, VH and VL amino acid sequences as a reference antibody's CDR, VH and VL sequences.

In exemplary aspects, the IgG1 antibody is an anti-EGFR antibody, e.g., an anti-HER2 monoclonal antibody. In exemplary aspects, the IgG1 antibody is trastuzumab, or a biosimilar thereof. The term trastuzumab refers to an IgG1 kappa humanized, monoclonal antibody that binds HER2/neu antigen (see CAS Number: 180288-69-1; DrugBank-DB00072; Kyoto Encyclopedia of Genes and Genomes (KEGG) entry D03257) comprising the VH and VL or VH-IgG1 and VL-IgG kappa sequences recited in Table 1 or set forth in SEQ ID Nos. 1-8, 21 or 22.

TABLE 1
Trastuzumab Amino Acid Sequences

| Description | Sequence | SEQ ID NO: |
|---|---|---|
| LC CDR1 | QDVNTA | 1 |
| LC CDR2 | SAS | 2 |
| LC CDR3 | QQHYTTPPT | 3 |
| HC CDR1 | GFNIKDTY | 4 |
| HC CDR2 | IYPTNGYT | 5 |
| HC CDR3 | SRWGGDGFYAMDY | 6 |
| VL | DIQMTQSPSSLSASVGDRVTITCRASQDVNTAVAWYQQKPGKAPKLL | 7 |
| | IYSASFLYSGVPSRFSGSRSGTDFTLTISSLQPEDFATYYCQQHYTTPPT | |
| | FGQGTKVEIK | |
| VH | EVQLVESGGGLVQPGGSLRLSCAASGFNIKDTYIHWVRQAPGKGLEW | 8 |
| | VARIYPTNGYTRYADSVKGRFTISADTSKNTAYLQMNSLRAEDTAVY | |
| | YCSRWGGDGFYAMDYWGQGTLVTVSS | |
| VL-IgG | DIQMTQSPSSLSASVGDRVTITCRASQDVNTAVAWYQQKPGKAPKLL | 21 |
| Kappa | IYSASFLYSGVPSRFSGSRSGTDFTLTISSLQPEDFATYYCQQHYTTPPT | |
| | FGQGTKVEIKRTVAAPSVFIFPPSDEQLKSGTASVVCLLNNFYPREAK | |
| | VQWKVDNALQSGNSQESVTEQDSKDSTYSLSSTLTLSKADYEKHKV | |
| | YACEVTHQGLSSPVTKSFNRGEC | |
| VH-IgG1 | EVQLVESGGGLVQPGGSLRLSCAASGFNIKDTYIHWVRQAPGKGLEW | 22 |
| | VARIYPTNGYTRYADSVKGRFTISADTSKNTAYLQMNSLRAEDTAVY | |
| | YCSRWGGDGFYAMDYWGQGTLVTVSSASTKGPSVFPLAPSSKSTSG | |
| | GTAALGCLVKDYFPEPVTVSWNSGALTSGVHTFPAVLQSSGLYSLSS | |
| | VVTVPSSSLGTQTYICNVNHKPSNTKVDKKVEPPKSCDKTHTCPPCPA | |
| | PELLGGPSVFLEPPKPKDTLMISRTPEVTCVVVDVSHEDPEVKFNWYV | |
| | DGVEVHNAKTKPREEQYNSTYRVVSVLTVLHQDWLNGKEYKCKVS | |
| | NKALPAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQ | |
| | GNVFSCSVMHEALHNHYTQKSLSLSPG | |

LC, light chain;
HC, heavy chain;
VL, variable light chain;
VH, variable heavy chain.

The trastuzumab antibody in some instances comprises (a) a light chain variable domain comprising: (i) a light chain CDR1 sequence comprising the amino acid sequence set forth in SEQ ID NO:1; (ii) a light chain CDR2 sequence comprising the amino acid sequence set forth in SEQ ID NO:2; and (iii) a light chain CDR3 sequence comprising the amino acid sequence set forth in SEQ ID NO:3; and (b) a heavy chain variable domain comprising: (i) a heavy chain CDR1 sequence comprising the amino acid sequence set forth in SEQ ID NO: 4; (ii) a heavy chain CDR2 sequence comprising the amino acid sequence set forth in SEQ ID NO:5, and (iii) a heavy chain CDR3 sequence comprising the amino acid sequence set forth in SEQ ID NO:6.

In alternative aspects, the IgG1 antibody is an anti-CD20 antibody, e.g., an anti-CD20 monoclonal antibody. In alternative aspects, the IgG1 antibody is rituximab, or a biosimilar thereof. The term rituximab refers to an IgG1 kappa chimeric murine/human, monoclonal antibody that binds CD20 antigen (see CAS Number: 174722-31-7; DrugBank-DB00073; Kyoto Encyclopedia of Genes and Genomes (KEGG) entry D02994) comprising the VH and VL or comprising VH-IgG1 and VL-IgG kappa sequences recited in Table 2 or set forth in SEQ ID Nos. 11-18, 23 or 24.

TABLE 2
Rituximab Amino Acid Sequences

| Description | Sequence | SEQ ID NO: |
|---|---|---|
| LC CDR1 | RASSSVSYIH | 11 |
| LC CDR2 | ATSNLAS | 12 |
| LC CDR3 | QQWTSNPPT | 13 |
| HC CDR1 | SYNMH | 14 |
| HC CDR2 | AIYPGNGDTSYNQKFKG | 15 |
| HC CDR3 | STYYGGDWYFNV | 16 |
| VL | QIVLSQSPAILSASPGEKVTMTCRASSSVSYIHWFQQKPGSSPKPWIYAT | 17 |
| | SNLASGVPVRFSGSGSGTSYSLTISRVEAEDAATYYCQQWTSNPRTFGG | |
| | GTKLEIK | |
| VL-IgG | QIVLSQSPAILSASPGEKVTMTCRASSSVSYHWFQQKPGSSPKPWIYAT | 23 |
| Kappa | SNLASGVPVRFSGSGSGTSYSLTISRVEAEDAATYCQQWTSNPPTFGG | |
| | GTKLEIKRTVAAPSVFIFPPSDEQLKSGTASVVCLLNNFYPREAKVQWK | |
| | VDNALQSGNSQESVTEQDSKDSTYSLSSTLTLSKADYEKHKVYACEVT | |
| | HQGLSSPVTKSFNRGEC | |
| VH | QVQLQQPGAELVKPGASVKMSCKASGYTFTSYNMHWVKQTPGRGLE | 18 |
| | WIGAIYPGNGDTSYNQKFKGKATLTADKSSSTAYMQLSSLTSEDSAVY | |
| | YCARSTYYGGDWYFNVWGAGTTVTVSA | |
| VH-IgG1 | QVQLQQPGAELVKPGASVKMSCKASGYTSYNMHWVKQTPGRGLE | 24 |
| | WIGAIYPGNGDTSYNQKFKGKATLTADKSSSTAYMQLSSLTSEDSAVY | |
| | YCARSTYYGGDWYFNVWGAGTTVTVSAASTKGPSVFPLAPSSKSTSG | |
| | GTAALGCLVKDYFPEPVTVSWNSGALTSGVHTFPAVLQSSGLYSLSSV | |
| | VTVPSSSLGTQTYICNVNHKPSNTKVDKKAEPKSCDKTHTCPPCPAPEL | |
| | LGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALP | |
| | APIEKTISKAKGQPREPQVYTLPPSRDELTKNQVSLTCLVKGFYPSDIAV | |
| | EWESNGQPENNYKTTPPVLDSDGSFFLYSKLTVDKSRWQQGNVFSCSV | |
| | MHEALHNHYTQKSLSLPGK | |

LC, light chain;
HC, heavy chain;
VL, variable light chain;
VH, variable heavy chain.

The rituximab antibody in some instances comprises (a) a light chain variable domain comprising: (i) a light chain CDR1 sequence comprising the amino acid sequence set forth in SEQ ID NO: 11; (ii) a light chain CDR2 sequence comprising the amino acid sequence set forth in SEQ ID NO: 12; and (iii) a light chain CDR3 sequence comprising the amino acid sequence set forth in SEQ ID NO: 13; and (b) a heavy chain variable domain comprising: (i) a heavy chain CDR1 sequence comprising the amino acid sequence set forth in SEQ ID NO: 14; (ii) a heavy chain CDR2 sequence comprising the amino acid sequence set forth in SEQ ID NO: 15, and (iii) a heavy chain CDR3 sequence comprising the amino acid sequence set forth in SEQ ID NO: 16.

### Additional Steps

The methods disclosed herein, in various aspects, comprise additional steps. For example, in some aspects, the methods comprise one or more upstream steps or downstream steps involved in producing, purifying, and formulating a recombinant protein, e.g., an antibody. In exemplary embodiments, the method comprises steps for generating host cells that express a recombinant glycosylated protein (e.g., antibody). The host cells, in some aspects, are prokaryotic host cells, e.g., E. coli or Bacillus subtilis, or the host cells, in some aspects, are eukaryotic host cells, e.g., yeast cells, filamentous fungi cells, protozoa cells, insect cells, or mammalian cells (e.g., CHO cells). Such host cells are described in the art. See, e.g., Frenzel, et al., Front Immunol 4: 217 (2013) and herein under “Cells.” For example, the methods comprise, in some instances, introducing into host cells a vector comprising a nucleic acid comprising a nucleotide sequence encoding the recombinant protein, or a polypeptide chain thereof.

In exemplary embodiments, the methods disclosed herein comprise steps for isolating and/or purifying the recombinant protein (e.g., recombinant antibody) from the culture. In exemplary aspects, the method comprises one or more chromatography steps including, but not limited to, e.g., affinity chromatography (e.g., protein A affinity chromatography), ion exchange chromatography, and/or hydrophobic interaction chromatography. In exemplary aspects, the method comprises steps for producing crystalline biomolecules from a solution comprising the recombinant proteins.

The methods of the disclosure, in various aspects, comprise one or more steps for preparing a composition, including, in some aspects, a pharmaceutical composition, comprising the purified recombinant protein. Such compositions are discussed below.

### Compositions

Provided herein are also compositions comprising recombinant glycosylated proteins and antibodies produced by the methods described herein. In exemplary embodiments, the compositions are prepared by methods which modulate the amount of glycans (e.g., galactosylated glycans, terminal β-galactose, G1, G1a, G1b and/or G2 galactosylated species, afucosylated glycans, core fucose, high mannose glycans, M5 high mannose species, or a combination thereof) in an antibody. In exemplary aspects, the recombinant glycosylated protein is an antibody. Accordingly, antibody compositions are provided herein, including IgG1 antibodies (e.g., trastuzumab or rituximab antibodies) having increased or decreased ADCP activity, wherein the IgG1 antibodies (e.g., trastuzumab or rituximab antibodies) have been engineered to have a specific ADCP activity or increased or decreased ADCP activity as compared to a control or reference antibody by modulating (e.g., increasing or decreasing) the amount of glycans (e.g., galactosylated glycans, terminal (3-galactose, G1, G1a, G1b and/or G2 galactosylated species, afucosylated glycans, core fucose, high mannose glycans, M5 high mannose species, or a combination thereof) on the IgG1 antibody (e.g., trastuzumab or rituximab antibodies).

In some embodiments, the composition comprises an IgG1 antibody (e.g., a trastuzumab or a rituximab antibody) produced by the methods described herein, wherein the IgG1 antibody (e.g., a trastuzumab or a rituximab antibody) has increased or decreased ADCP activity compared to a reference IgG1 antibody (e.g., a trastuzumab or a rituximab antibody) having the same antibody sequence as the IgG1 antibody (e.g., a trastuzumab or a rituximab antibody) having increased or decreased ADCP activity.

Accordingly, in exemplary embodiments, the presently disclosed antibody compositions have increased ADCP activity to any degree or level relative to a control or a reference antibody. In exemplary instances, the increased ADCP activity of the antibody compositions disclosed herein using the methods of the disclosure is at least or about a 1% to about a 100% increase (e.g., at least or about a 1% increase, at least or about a 2% increase, at least or about a 3% increase, at least or about a 4% increase, at least or about a 5% increase, at least or about a 6% increase, at least or about a 7% increase, at least or about a 8% increase, at least or about a 9% increase, at least or about a 9.5% increase, at least or about a 9.8% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, at least or about a 100% increase) relative to a control or a reference antibody. In exemplary embodiments, the increased ADCP activity of the antibody compositions disclosed herein using the methods of the disclosure is over 100%, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to a control or a reference antibody. In exemplary embodiments, the level of ADCP activity of the antibody or composition comprising the same increases by at least about 1.5-fold, relative to a control or a reference antibody. In exemplary embodiments, the level of ADCP activity of the antibody or composition comprising the same increases by at least about 2-fold, relative to a control or a reference antibody. In exemplary embodiments, the level of ADCP activity of the antibody or composition comprising the same increases by at least about 3-fold, relative to a control or a reference antibody. In exemplary embodiments, the level of ADCP activity of the antibody or composition comprising the same increases by at least about 4-fold or about 5-fold, relative to a control or a reference antibody. In exemplary embodiments, the level of ADCP activity of the antibody or composition comprising the same increases by at least about 6-fold, about 7-fold, about 8-fold, about 9-fold, or about 10-fold, relative to a control or a reference antibody. In exemplary embodiments, the level of ADCP activity of the antibody or composition comprising the same increases by an amount falling within the range of about 0.5-fold to about 8-fold, relative to a control or a reference antibody.

In alternative embodiments, the presently disclosed antibody compositions have decreased ADCP activity to any degree or level relative to a control or a reference antibody. For example, the decreased ADCP activity of the antibody compositions disclosed herein using the methods of the disclosure is at least or about a 1% to about a 100% decrease (e.g., at least or about a 1% decrease, at least or about a 2% decrease, at least or about a 3% decrease, at least or about a 4% decrease, at least or about a 5% decrease, at least or about a 6% decrease, at least or about a 7% decrease, at least or about a 8% decrease, at least or about a 9% decrease, at least or about a 9.5% decrease, at least or about a 9.8% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, at least or about a 100% decrease) relative to the level of a control or a reference antibody. In exemplary embodiments, the decreased ADCP activity of the antibody compositions disclosed herein using the methods of the disclosure is over about 100%, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to the level of a control or a reference antibody. In exemplary embodiments, the level of ADCP activity of the antibody, or composition comprising the same decreases by at least or about 1.5-fold, relative to a control or a reference antibody. In exemplary embodiments, the level of ADCP activity of the antibody, or composition comprising the same decreases by at least about 2-fold, relative to a control or a reference antibody. In exemplary embodiments, the level of ADCP activity of the antibody, or composition comprising the same decreases by at least about 3-fold, relative to a control or a reference antibody. In exemplary embodiments, the level of ADCP activity of the antibody, or composition comprising the same decreases by at least about 4-fold or by at least about 5-fold, relative to a control or a reference antibody. In exemplary embodiments, the level of ADCP activity of the antibody or composition comprising the same decreases by at least about 6-fold, about 7-fold, about 8-fold, about 9-fold, or about 10-fold, relative to a control or a reference antibody. In exemplary embodiments, the level of ADCP activity of the antibody or composition comprising the same decreases by an amount falling within the range of about 0.5-fold to about 8-fold, relative to a control or a reference antibody.

In exemplary aspects, the antibody compositions of the present disclosure include antibodies having an increased amount of glycans (e.g., galactosylated glycans, G1, G1a, G1b and/or G2 galactosylated species, afucosylated glycans, core fucose, high mannose glycans, M5 high mannose species, or a combination thereof) to any degree or level relative to a control or a reference antibody. In exemplary instances, the antibody compositions have an increased amount of glycans, wherein the glycans are increased by at least or about 1% to about 100% (e.g., at least or about 1%, at least or about 2%, at least or about 3%, at least or about 4%, at least or about 5%, at least or about 6%, at least or about 7%, at least or about 8%, at least or about 9%, at least or about 9.5%, at least or about 9.8%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 100%) relative to a control or a reference antibody. In exemplary embodiments, the antibody compositions have an increased amount of glycans, wherein the glycans are increased by 100% or more, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to a control or a reference antibody. In exemplary embodiments, the antibody compositions have an increased amount of glycans, wherein the glycans are increased by at least about 1.5-fold, relative to a control or a reference antibody. In exemplary embodiments, the antibody compositions have an increased amount of glycans, wherein the glycans are increased by at least about 2-fold, relative to a control or a reference antibody. In exemplary embodiments, the antibody compositions have an increased amount of glycans, wherein the glycans are increased by at least about 3-fold, relative to a control or a reference antibody. In exemplary embodiments, the antibody compositions have an increased amount of glycans, wherein the glycans are increased by at least about 4-fold or about 5-fold, relative to a control or a reference antibody. In exemplary embodiments, the antibody compositions have an increased amount of glycans, wherein the glycans are increased by at least about 6-fold, about 7-fold, about 8-fold, about 9-fold, or about 10-fold, relative to a control or a reference antibody. In exemplary embodiments, the antibody compositions have an increased amount of glycans, wherein the glycans are increased by an amount falling within the range of about 0.5-fold to about 8-fold, relative to a control or a reference antibody.

In exemplary aspects, the compositions of the present disclosure include antibodies having a reduced amount of glycans (e.g., galactosylated glycans, G1, G1a, G1b and/or G2 galactosylated species, afucosylated glycans, core fucose, high mannose glycans, M5 high mannose species, or a combination thereof) to any degree or level relative to a control or a reference antibody. In exemplary instances, the antibody compositions have a reduced amount of glycans, wherein the glycans are reduced by at least or about 1% to about 100% (e.g., at least or about 1%, at least or about 2%, at least or about 3%, at least or about 4%, at least or about 5%, at least or about 6%, at least or about 7%, at least or about 8%, at least or about 9%, at least or about 9.5%, at least or about 9.8%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 100%) relative to a control or a reference antibody. In exemplary embodiments, the antibody compositions have a reduced amount of glycans, wherein the glycans are reduced by 100% or more, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to a control or a reference antibody. In exemplary embodiments, the antibody compositions have a reduced amount of glycans, wherein the glycans are reduced by at least about 1.5-fold, relative to a control or a reference antibody. In exemplary embodiments, the antibody compositions have a reduced amount of glycans, wherein the glycans are reduced by at least about 2-fold, relative to a control or a reference antibody. In exemplary embodiments, the antibody compositions have a reduced amount of glycans, wherein the glycans are reduced by at least about 3-fold, relative to a control or a reference antibody. In exemplary embodiments, the antibody compositions have a reduced amount of glycans, wherein the glycans are reduced by at least about 4-fold or about 5-fold, relative to a control or a reference antibody. In exemplary embodiments, the antibody compositions have a reduced amount of glycans, wherein the glycans are reduced by at least about 6-fold, about 7-fold, about 8-fold, about 9-fold, or about 10-fold, relative to a control or a reference antibody. In exemplary embodiments, the antibody compositions have a reduced amount of glycans, wherein the glycans are reduced by an amount falling within the range of about 0.5-fold to about 8-fold, relative to a control or a reference antibody.

In exemplary aspects, the antibody compositions of the present disclosure comprise a total amount of glycans (e.g., galactosylated glycans, G1, G1a, G1b and/or G2 galactosylated species, afucosylated glycans, core fucose, high mannose glycans, M5 high mannose species, or a combination thereof) of at least or about 0.5%, at least or about 1%, at least or about 2%, at least or about 3%, at least or about 5%, at least or about 7%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97% or at least or about 98% or increased or decreased to a total amount in the range of at least or about 0.5% to 98%.

In exemplary embodiments, the antibody compositions of the present disclosure comprise a trastuzumab antibody having an increased amount of terminal β-galactose or amount of G1, G1a, G1b and/or G2 galactosylated species relative to a control or reference trastuzumab antibody and an increased ADCP activity relative to a control or reference trastuzumab antibody. In some embodiments, the antibody compositions of the present disclosure comprise a trastuzumab antibody, wherein about a 1 percent increase in the amount of terminal β-galactose or amount of G1, G1a, G1b and/or G2 galactosylated species relative to a control or reference trastuzumab antibody results in an increased ADCP activity of about 2.5, about 2.8, about 2.88 or about 3 percent relative to a control or reference trastuzumab antibody.

In exemplary embodiments, the antibody compositions of the present disclosure comprise a trastuzumab antibody having a decreased amount of terminal β-galactose or amount of G1, G1a, G1b and/or G2 galactosylated species relative to a control or reference trastuzumab antibody and a decreased ADCP activity relative to a control or reference trastuzumab antibody. In some embodiments, the antibody compositions of the present disclosure comprise a trastuzumab antibody, wherein about a 1 percent decrease in the amount of terminal β-galactose or amount of G1, G1a, G1b and/or G2 galactosylated species relative to a control or reference trastuzumab antibody results in a decreased ADCP activity of about 2.5, about 2.8, about 2.88 or about 3 percent relative to a control or reference trastuzumab antibody.

In exemplary embodiments, the antibody compositions of the present disclosure comprise a rituximab antibody having a decreased amount of afucosylated glycans or an increased the amount of core fucose relative to a control or reference rituximab antibody and an increased ADCP activity relative to a control or reference rituximab antibody. In some embodiments, the antibody compositions of the present disclosure comprise a rituximab antibody, wherein about a 1 percent decrease in afucosylated glycans or a 1 percent increase in core fucose results in an increased ADCP activity of about 0.5, about 0.7, about 0.75, about 0.8 or about 1 percent relative to a control or reference rituximab antibody.

In exemplary embodiments, the antibody compositions of the present disclosure comprise a trastuzumab antibody having an increased amount of afucosylated glycans or a decreased amount of core fucose relative to a control or reference trastuzumab antibody and an increased ADCP activity relative to a control or reference trastuzumab antibody. In some embodiments, the antibody compositions of the present disclosure comprise a trastuzumab antibody, wherein about a 1 percent increase in afucosylated glycans or a 1 percent decrease in core fucose results in an increased ADCP activity of about 0.5, about 0.56, about 0.6 or about 1 percent relative to a control or reference trastuzumab antibody.

In exemplary embodiments, the antibody compositions of the present disclosure comprise a trastuzumab antibody having a decreased amount of afucosylated glycans or an increased amount of core fucose relative to a control or reference trastuzumab antibody and a decreased amount of ADCP activity relative to a control or reference trastuzumab antibody. In some embodiments, the antibody compositions of the present disclosure comprise a trastuzumab antibody, wherein about a 1 percent decrease in afucosylated glycans or a 1 percent increase in core fucose results in decreased ADCP activity of about 0.5, about 0.56, about 0.6 or about 1 percent relative to a control or reference trastuzumab antibody.

In exemplary embodiments, the antibody compositions of the present disclosure comprise a rituximab antibody having an increased the amount of afucosylated glycans or decreased the amount of core fucose relative to a control or reference rituximab antibody and a decreased ADCP activity relative to a control or reference rituximab antibody. In some embodiments, the antibody compositions of the present disclosure comprise a rituximab antibody, wherein about a 1 percent increase in afucosylated glycans or a 1 percent decrease in core fucose results in a decreased ADCP activity of about 0.5, about 0.7, about 0.75, about 0.8 or about 1 percent relative to a control or reference rituximab antibody.

In exemplary embodiments, the antibody compositions of the present disclosure comprise an IgG1 antibody (including, e.g., trastuzumab or rituximab) having an increased amount of high mannose glycans, including, e.g., M5 high mannose species, relative to a control or reference IgG1 antibody (including, e.g., trastuzumab or rituximab) and a decreased ADCP activity relative to a control or reference IgG1 antibody (including, e.g., trastuzumab or rituximab). In some embodiments, the antibody compositions of the present disclosure comprise an IgG1 antibody (including, e.g., trastuzumab or rituximab), wherein about a 1 percent increase in high mannose glycans, including, e.g., M5 high mannose species, results in a decreased ADCP activity of about 1%, about 1.2%, about 1.31%, about 1.5%, about 1.7%, about 2%, about 2.11% or about 2.5%. relative to a control or reference IgG1 antibody (including, e.g., trastuzumab or rituximab).

In exemplary embodiments, the antibody compositions of the present disclosure comprise an IgG1 antibody (including, e.g., trastuzumab or rituximab) having a decreased amount of high mannose glycans, including, e.g., M5 high mannose species, relative to a control or reference IgG1 antibody (including, e.g., trastuzumab or rituximab) and an increased ADCP activity relative to a control or reference IgG1 antibody (including, e.g., trastuzumab or rituximab). In some embodiments, the antibody compositions of the present disclosure comprise an IgG1 antibody (including, e.g., trastuzumab or rituximab), wherein about a 1 percent decrease in high mannose glycans, including, e.g., M5 high mannose species, results in an increased ADCP activity of about 1%, about 1.2%, about 1.31%, about 1.5%, about 1.7%, about 2%, about 2.11% or about 2.5%. relative to a control or reference IgG1 antibody (including, e.g., trastuzumab or rituximab).

In some additional exemplary embodiments, the antibody compositions of the present disclosure comprise an IgG1 antibody (including, e.g., trastuzumab or rituximab) having an increased or decreased amount of a combination of galactosylated glycans (including, e.g., terminal β-galactose or G1, G1a, G1b and/or G2 galactosylated species), afucosylated glycans or core fucose, and/or high mannose glycans (including, e.g., M5 high mannose species). In exemplary embodiments, the antibody compositions comprise an IgG1 antibody (including, e.g., trastuzumab or rituximab) having: (1) an increased amount of terminal β-galactose or G1, G1a, G1b and/or G2 galactosylated species; (2) a decreased amount of core fucose or an increased amount of afucosylated species; and/or (3) a decreased amount of high mannose or M5 high mannose species; relative to a control or reference IgG1 antibody (including, e.g., trastuzumab or rituximab) resulting in an increased ADCP activity relative to a control or reference IgG1 antibody (including, e.g., trastuzumab or rituximab). In some other exemplary embodiments, the antibody compositions comprise an IgG1 antibody (including, e.g., trastuzumab or rituximab) having: (1) a decreased amount of terminal β-galactose or G1, G1a, G1b and/or G2 galactosylated species; (2) an increased amount of core fucose or a decreased amount of afucosylated species; and/or (3) an increased amount of high mannose or M5 high mannose species; relative to a control or reference IgG1 antibody (including, e.g., trastuzumab or rituximab) resulting in a decreased ADCP activity relative to a control or reference IgG1 antibody (including, e.g., trastuzumab or rituximab).

In exemplary embodiments, the antibody compositions provided herein are combined with a pharmaceutically acceptable carrier, diluent or excipient. Accordingly, provided herein are pharmaceutical compositions comprising the recombinant glycosylated protein composition (e.g., the antibody composition) described herein and a pharmaceutically acceptable carrier, diluent or excipient. As used herein, the term “pharmaceutically acceptable carrier” includes any of the standard pharmaceutical carriers, such as a phosphate buffered saline solution, water, emulsions such as an oil/water or water/oil emulsion, and various types of wetting agents.

The following examples are given merely to illustrate the present disclosure and not in any way to limit its scope.

### EXAMPLES

The following Examples describe modulating ADCP effector function of IgG1 antibodies, including anti-HER2 and anti-CD20 antibodies, through the increase or decrease of specific glycans, including β-galactose, core fucose/afucosylation and/or high mannose.

### Introduction

Therapeutic monoclonal antibodies, particularly of the IgG1 subclass, are capable of effector function activities that may be important for their mechanism of action. One such effector function activity is Antibody Dependent Cellular Phagocytosis (ADCP), which has been shown to be mediated primarily through the activating FcγR, FcγRIIa, on macrophages and neutrophils. The critical quality attributes that are the most impactful and predictive of ADCP activity, and therefore most suitable to monitor during IgG1 antibody manufacturing, are not well established. Primary cell assays for ADCP are often laborious and subject to donor to donor variability, making such assays less desirable for product characterization. By developing and employing an ADCP reporter gene assay, we have been able to determine with high sensitivity the glycan structures that can impact FcγRIIa-mediated ADCP across multiple different IgG1 mAbs. Interestingly we observed that some IgG1 antibodies are very potent mediators of ADCP while others do not mediate ADCP even though they possess other effector function activities (ADCC and CDC). Additionally, we find that ADCP by different IgG1 mAbs has markedly different sensitivity to glycan species, with one mAb demonstrating a surprisingly strong influence of β-galactosylation and high mannose levels.

It has been recognized that FcγRIIa mediated ADCP may play a role for the mechanism of action (MoA) of some therapeutic Mabs. Weng W K, Levy R. Two immunoglobulin G fragment C receptor polymorphisms independently predict response to rituximab in patients with follicular lymphoma. Journal of clinical oncology: official journal of the American Society of Clinical Oncology 2003; 21:3940-7; Zhang W, et al. FCGR2A and FCGR3A polymorphisms associated with clinical outcome of epidermal growth factor receptor expressing metastatic colorectal cancer patients treated with single-agent cetuximab. Journal of clinical oncology: official journal of the American Society of Clinical Oncology 2007; 25:3712-8. With this appreciation comes the need for suitable in vitro assays with sufficient resolution and discriminatory power. One possible reason for the lack of clear understanding of the impact of glycan structures in ADCP may be the challenges in performing the necessary functional assays. Most published ADCP protocols use primary effector cells and fluorescent markers to follow activity using flow cytometry (see, e.g., Petricevic B, et al. Trastuzumab mediates antibody-dependent cell-mediated cytotoxicity and phagocytosis to the same extent in both adjuvant and metastatic HER2/neu breast cancer patients. Journal of translational medicine 2013; 11:307). Such assays can be indicative of directional effects but can have limited quantitative power and lack repeatability due to donor to donor variability and limited response levels. And even though it can be argued that the use of primary cells offers a more physiologically relevant assessment compared to cell line based or receptor binding assays, they may not be sensitive or precise enough to reveal the potential for effector function activity. An FcγRIIa reporter gene assay has recently been described and offers the promise of greatly simplified performance with high precision and accuracy. See Tada M, et al. Development of a cell-based assay measuring the activation of FcgammaRIIa for the characterization of therapeutic monoclonal antibodies. PloS one 2014; 9:e95787. We've established a similar assay derived from a commercially available source that makes use of a reporter gene cell line responsive to FcγRIIa engagement. The assay is highly precise, reproducible and has good sensitivity to detect the contributions of different glycoforms. With the recognition that the Fc glycan structures can require close process monitoring to control during the manufacturing process, it's possible to encounter a relatively high degree of heterogeneity even in products from well-developed manufacturing processes. See Read E K, et al. Industry and regulatory experience of the glycosylation of monoclonal antibodies. Biotechnology and applied biochemistry 2011; 58:213-9. Therefore, it is essential to establish a robust model for the contributions of different glycan structures and ADCP activity. Here we describe the results from the engineered ADCP assay in combination with FcγR binding studies to characterize the impact of a wide range of glycan types and levels across multiple IgG1 monoclonal antibodies exhibiting effector function.

In the following Examples, the following methods were used.

### Materials and Methods

### Sample Preparation

Humanized monoclonal IgG1 antibodies were expressed in CHO cells and produced at Amgen (Thousand Oaks, Calif.). The IgG1 antibodies (“mAbs”) used in this study bind to membrane expressed targets (CD20, HER2 or TNFα), and include trastuzumab (anti-HER2) and rituximab (anti-CD20).

### Enrichment and Enzymatic Remodeling of Glycan Species

High mannose containing species were enriched from mAbs using ProSwift ConA-1S affinity column (5×50 mm, ThermoFisher, PN 074148) on an Agilent 1100 series HPLC system with a flow rate of 0.5 mL/min. The column was first kept at initial condition with 100% buffer A (50 mM sodium acetate, 0.2M NaCl, 1 mM CaCl2, 1 mM MgCl2, pH 5.3) for 10.5 min, and then eluted with 100% buffer B (50 mM sodium acetate, 0.2 M NaCl, 1 mM CaCl2, 1 mM MgCl2. 100 mM α-methyl-mannopyranoside pH 5.3) for 17.5 min. Both flow-through and eluted fractions were collected and treated with β-(1-4)-Galactosidase (QA Bio, PN E-BG07) to remove terminal galactose. Specifically, mAb fractions were incubated with β-(1-4)-galactosidase at a ratio of 1/50 (μg/μg) in the presence of a reaction buffer containing 50 mM sodium phosphate (pH 6.0) for 1 hour at 37° C. Reactions were terminated by flash freezing.

Afucosylated species were prepared from mAbs by enzymatic treatment with Endo-H (QA-Bio, PN E-EH02). Specifically, mAbs were incubated with Endo-H for 24 hours at 37° C. in a reaction buffer of 50 mM sodium phosphate (pH 5.5). The final mAb concentration is 4 mg/mL. Subsequently, afucosylated mAbs were separated by affinity chromatography using customized glycap-3A column (low density FcγIIIa, 3×150 mm, Zepteon, PN R3AVD1P1ML) on an Agilent 1100 series HPLC. The mobile phase A contained 20 mM Tris, 150 mM NaCl, pH 7.5 and the mobile phase B was 50 mM sodium citrate (pH 4.2). A gradient (hold at 0% B for 8 min, 0% to 18% B for 22 min) at a flow rate of 0.5 mL/min was used to separate both afucose-depleted (flow-through) and enriched (eluate) mAbs. Enzymatic treatment with β-(1-4)-galactosidase (as described above) was also carried out to remove any potential impact from terminal galactose.

Galactose remodeled samples were generated through the in vitro activity of β-1,4-galactosyltransferase (Sigma/Roche). First, fucosylated mAbs (mainly G0F) were prepared by collecting the flow-through fraction from FcγIIIa column and treated with galactosidase to remove terminal Galactose. Then, G0F enriched mAbs were incubated with β-1,4-galactosyltransferase at 37° C. in a reaction buffer containing 10 mM UDP-galactose, 100 mM MES (pH 6.5), 20 mM MnCl2 and 0.02% sodium azide. The final enzyme to mAb ratio is 6 (μL/mg) with a mAb concentration of 2 mg/mL. MAbs with different level of galactose were obtained by taking sample out of the reaction mixture at different time points (10 min, 20 min, 30 min, 1 hr, 2 hr, 4 hr and 9 hr) followed by flash freezing to halt the reaction.

Protein A chromatography purification was performed for all the enriched and remodeled samples to remove enzymes and other components. The purification was carried out with a prepacked protein A column (Poros A/20, 4.6×100 mm, Applied Biosystems, PN 1-5022-26) on an Agilent 1100 series HPLC system with a flow rate of 3 mL/min. After loading the appropriate amount of each sample, the column was first kept at initial condition with 100% buffer A (20 mM Tris-HCl/150 mM NaCl, pH 7.0) for 1.4 min, and then eluted with 100% buffer B (0.1% acetic acid) for 2.9 min. All eluted mAbs were diafiltered into formulation buffer using Amicon Ultra centrifugal filters with a 3 kDa cutoff membrane. Protein concentration was typically ˜1 mg/mL for all enriched/remodeled mAb samples.

### Characterization of Enrichment and Remodeling of Glycan Species.

All the enriched and remodeled samples were characterized with Hydrophilic Interaction Liquid Chromatography (HILIC) and Size Exclusion Chromatography to ensure desired glycan properties and minimal level of high molecular weight species. Glycans from mAbs were released using PNGase F (New England BioLabs) with an E/S ratio of 1/25 (4/μg) and labeled with 12 mg/mL 2-aminobenzoic acid (2-AA, Sigma-Aldrich) by incubating the reaction mixture at 80° C. for 75 min. 2-AA labeled glycans were separated with BEH glycan column (1.7 μm, 2.1×100 mm, Waters) on a Waters Acuity or H-Class UPLC system equipped with a fluorescence detector. The column temperature was maintained at 55° C. The mobile phase A contained 100 mM ammonium format (pH3.0) and the mobile phase B was 100% acetonitrile. Glycans were bound to the column in high organic solvent then eluted with an increasing gradients of aqueous ammonium formate buffer (76% B was held for 5 min, followed by a gradient from 76 to 65.5% B over 14 min). Confirmation that the required manipulations didn't result in the formation of high molecular weight species was assessed using a size exclusion column (SEC) TSK-Gel G3000SWLXL (7.8×300 mm, Tosoh Bioscience) on an Agilent 1100 HPLC system with a flow rate of 0.5 mL/min. Sample loads of 20-40 μg of sample were typically separated isocratically with a mobile phase containing 100 mM sodium phosphate (pH 6.8) and 250 mM NaCl.

### ADCP Assay

The ADCP luciferase reporter gene assay employs engineered Jurkat T cells as the effector cells. The Jurkat reporter cells express FcγRIIa-(H131 variant) on the cell surface as well as a luciferase reporter gene with a response element for the nuclear factor of activated T cell (NFAT). These cells were adapted from Promega Catalogue # G9901. Simultaneous binding between an antibody bound to target cells and to the FcγRIIa on Jurkat effector cells activates the transcription factor NFAT. Activated NFAT translocates into the nucleus of Jurkat cells and induces luciferase reporter gene expression. After addition of a luciferase substrate that contains luciferin and surfactant, luminescence signal generation enables detection of ADCP reporter activity. For anti CD20 antibody, the reference standard, assay control, and test samples were serial diluted over 8 concentration levels in RPMI 1640 assay medium with low IgG FBS to the range of 1.76-600 ng/mL of the final plate well concentration to serve as a dose response curve. Effector Jurkat reporter and target (CD 20+ Raji) cells were prepared in a combined cell suspension at an effector to target (E to T) cell ratio of 3:2. The plate was then incubated in a humidified incubator at 5% CO2 and 37° C. for 5.5 hours. At the end of the incubation, cells are lysed by the surfactant in the luciferase assay buffer. Luminescence was generated via luciferase catalysis of its substrate luciferin and was detected by an EnVision plate reader. Data were fitted to the mean emission values using a 4 parameter curve fit using SoftMax Pro 7 and reported as percent relative activity (EC50 standard/EC50 sample) per common bioassay practices. Each sample was tested in 3 independent assays and the sample final result is reported as the mean of the 3 determinations. The anti-TNFα antibody assay was also performed similarly but used a 234.4-30000 ng/mL dose range and a CHO target cell line engineered to express a non-cleavable form of TNFα on its cell surface15. The ADCP assay for the anti HER2 antibody was carried out similarly but had a dose response range of 31.3-4000 ng/mL and used the HER2+ SKOV-3 cell line as target cells. The plate incubation at 5% CO2 and 37° C. is around 6.5 hours to achieve higher luciferase response. The activity for the anti-HER2 antibody was calculated by fitting the dose-response data to a 4 parameter curve similar to the other molecules, but rather that using SoftMax Pro to calculate a relative activity, the following empirically derived equation was used: Relative Potency=(B sample/B reference)E×(C reference/C sample)×(D sample/D reference)F. The B ratio (B sample/B reference) was obtained by dividing the hill slope of the reference standard by that of the sample. The C ratio (C reference/C sample) was obtained by dividing the EC50 of the reference standard by that of the sample with values. The D ratio (D sample/D reference) was obtained by dividing the upper asymptote of the reference standard by that of the sample with values. If B sample/B reference ≥1.0 & C reference/C sample ≥1.0, E=0.05; If B sample/B reference ≥1.0 & C ref/C sample <1.0, E=0.20; If B sample/B reference <1.0, E=0.15; If D reference/D sample >1.0, F=1.8×(D reference/D sample) (0.45×(D reference/D sample)\hat{ }0.35); If D reference/D sample ≤1.0, F=1.8×(D sample/D reference) (1.45×(D reference/D sample)\hat{ }1.75). This equation considered the fact the B, C and D values are all impacted by quality attributes in this assay.

### FcγRIIa Binding.

Relative binding activity to FcγRIIa by the antibody samples was measured in a competitive amplified luminescent proximity homogeneous assay (AlphaLISA™, Perkin Elmer). The assay contains 2 bead types, an acceptor bead and a donor bead. The acceptor beads contain glutathione, which binds recombinant human FcγRIIa-glutathione-S transferase (FcγRIIa GST) prepared in house. The donor beads were coated with streptavidin, which binds to a biotinylated human IgG1 prepared in house, which serves as a competitor for the samples. When Fc□RIIa GST and the biotinylated human IgG1 bind together, they bring the acceptor and donor beads into close proximity. When a laser is applied to this complex, ambient oxygen is converted to singlet oxygen by the donor bead leading to light emission measured at 570 nm. When sample is present at sufficient concentrations to inhibit the binding of FcγRIIa GST to the biotinylated human IgG1, a dose dependent decrease in emission is measured. The binding assay was performed in a 96-well AlphaPlate (PerkinElmer). Glutathione-coated acceptor beads were coated with human Fc gamma RIIa-GST-H6 and prepared to a final concentration of 25 μg/mL and 5 nM in AlphaLISA Immunoassay buffer, respectively. This mixture is incubated for 2 to 4 hours at room temperature in the dark (beads are light sensitive). Reference standard, control, and sample material are serially diluted 2.5-fold from 100 nM to 0.164 nM in (PerkinElmer) in AlphaLISA™ immunoassay buffer in a dilution plate, and 90 μL of each well is transferred to a fresh mixing plate. The biotinylated IgG1 competitor is prepared to a final concentration of 2 nM in immunoassay buffer. Ninety μL of biotinylated IgG1 competitor was added to all wells of the mixing plate from low to high concentration. Each well was mixed at least 5× by pipetting up and down, and then 40 μL was transferred to each appropriate well of the three replicate assay plates. After the two hour incubation of glutathione acceptor beads and FcγRIIa was complete, 40 μL of the mixture was added to each well of the assay plates. Plates were sealed and placed on a shaker for 2 minutes, then incubated in the dark for 22 to 26 hours at room temperature. Following incubation, streptavidin donor beads were diluted to a final concentration of 50 μg/mL in immunoassay buffer, and 20 μL was added to each well of the assay plates, for a total reaction volume of 100 μL. The plates were again sealed, placed on a shaker for 2 minutes, and incubated in the dark for 2-6 hours at room temperature. Following incubation, plate sealers were removed, and the plates were read on the Envision at 680 nm excitation, 570 nm emission. The test sample binding relative to a reference standard was determined and reported as percent relative binding. Each data point of the dilution curve was run in triplicate across three assay plates. Data were fitted to the mean emission values using a 4-parameter curve fit using SoftMax Pro 7 and reported as percent activity as calculation by IC50 standard/IC50 sample. Each sample was tested in 3 independent assays, and the sample final result was reported as the mean of the 3 determinations.

### Antigen Binding

The CD20 antigen binding assay was performed with WIL2-S cells, a human β-lymphoblastoid cell line, utilizing a competitive assay format reporting fluorescence inhibition. The test sample competes with a fixed concentration of an Alexa-488 labeled form of the reference standard for binding to the cell surface expressed CD20 on WIL2-S cells. Dose response curves were generated for the reference standard, assay control and test samples by serially diluting over 8 concentrations in PBS containing 0.5 mg/mL BSA to a final concentration range of 4.92-3000 ng/mL. The Alexa-488 labeled competitor was diluted to final in-well concentration of 100 ng/mL. Sample, competitor and WIL2-S cells diluted to 30,000 cells/well were added to a 96 well plate in duplicate and sealed with a plate sealer. The plates were then incubated for 4.5-6 hours at room temperature prior to measuring the fluorescence signal with an Acumen® eX3 imaging cytometer (TTP Labtech). A dose dependent decrease in fluorescence signal was detected with the increasing concentration of test sample. The relative CD20 binding of the test sample was reported relative to a reference standard sample. Data were fitted to the mean emission values using a 4-parameter curve fit using SoftMax Pro 7 and reported as percent relative binding activity was calculated by IC50 standard/IC50 sample. Each sample was tested in 3 independent assays, and the final result were reported as the mean of the 3 determinations. HER2 binding assays were performed in a similar fashion with the exception that the HER2 positive SKBR-3 were used as target cells and the dose response curve ranged from (0.016-10 μg/mL).

### Example 1

### Assessment of ADCP Activity of Anti-HER2, Anti-CD20 and Anti-TNFα IgG1 Antibodies

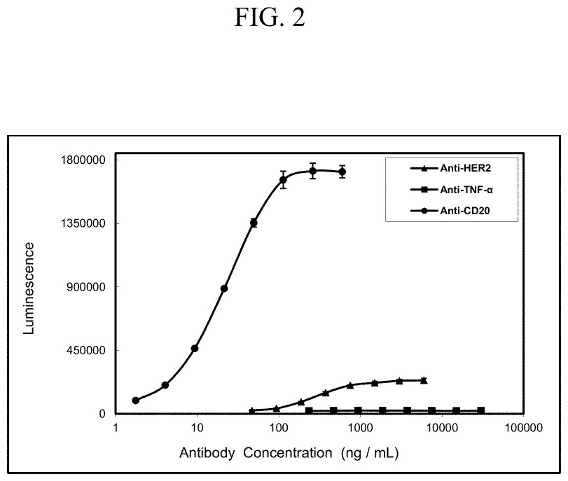

Assessment of potential ADCP activity for a series of human IgG1 monoclonal antibodies to cell surface antigens was undertaken using the ADCP reporter gene assay (see materials and methods above for details). The antibodies assessed displayed a range of activities in this assay. Overlays of the dose response curves for three different IgG1 mAbs representing a range of activities are shown in FIG. 2. The anti-TNFα antibody showed virtually no response even though it was assessed at the highest concentration range tested of all the antibodies. In contrast, the anti-CD20 antibody showed a very robust response with a 20-fold min to max stimulation over a relatively low concentration range. The anti-HER2 antibody shows an intermediate response. All three of these antibodies mediate ADCC on these same target cell lines as measured by an in-vitro ADCC activity assay (data not shown). With the observation there was ADCP activity obtained by two different antibodies to two different targets, a more detailed study of the assay and attributes that impact these activities was undertaken.

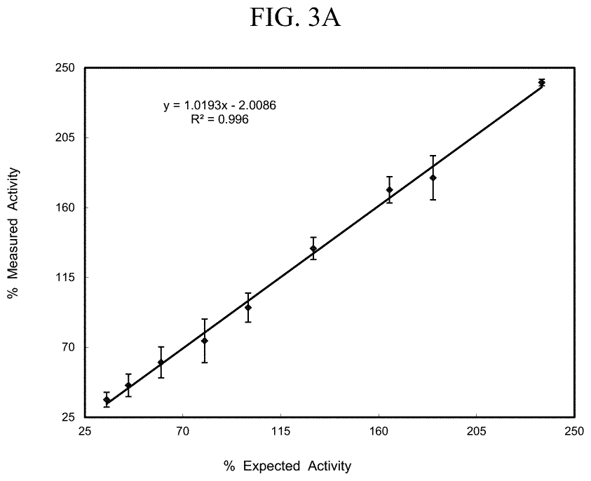

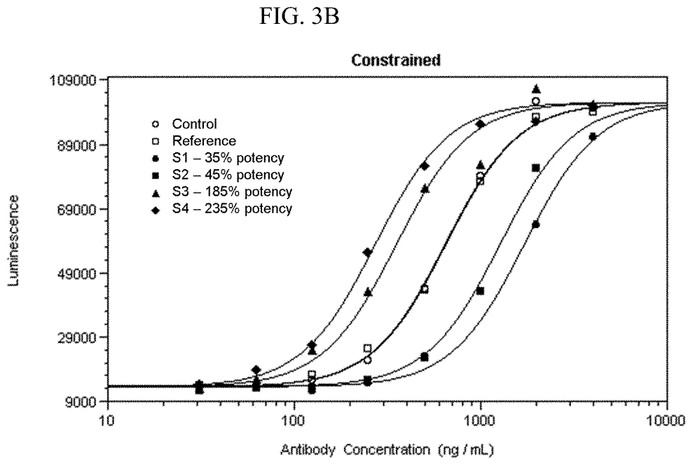

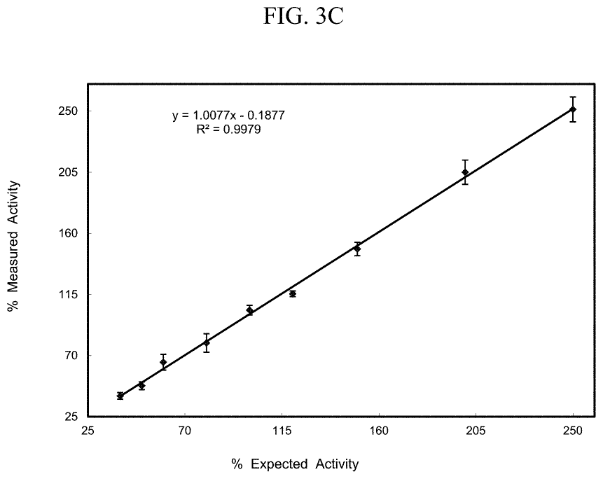

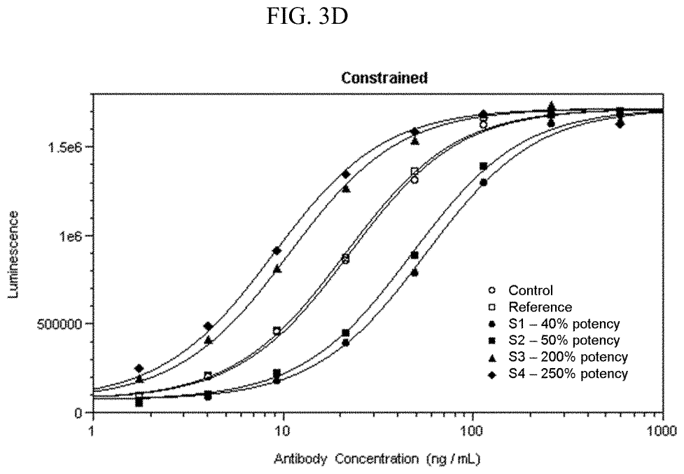

As a first step, to demonstrate the quantitative nature and range of the ADCP reporter gene assay, and thus its suitability for the assessment of quality attributes that would impact ADCP activity, linearity assays were undertaken using simulated activity or potency levels by concentration adjustment. FIGS. 3A-D show the results of the expected vs observed ADCP activity measurements for both the anti CD20 (FIGS. 3A-B) and anti-HER2 (FIGS. 3C-D) antibodies, with the respective dose response curves (see FIGS. 3B and 3D) and the correlation graphs (see FIGS. 3A and 3C).

With the quantitative ranges for the assay established from 25% to 250% relative ADCP activity, we next examined the quality attributes associated with the conserved N-linked glycan in the Fc domain of the two active mAbs (anti-CD20 and anti-HER2). Samples of each antibody were prepared to evaluate a range of high mannose, beta-galactose and fucose structures as shown in FIG. 4.

Antibody samples possessing a range of each of the major glycan species were prepared as described. The impact of each of the glycans on the relative ADCP activity for are detailed in the following sections.

### Example 2

### Impact of Terminal β-Galactose on ADCP Activity of Anti-HER2 and Anti-CD20 IgG1 Antibodies

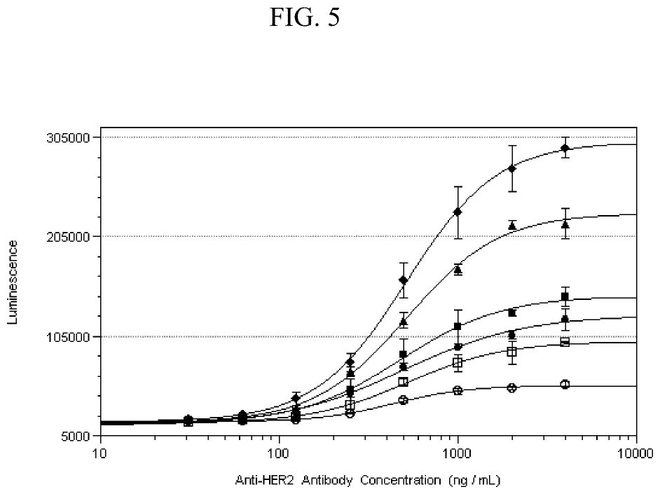

The first glycan species examined was β-galactose. The anti-HER2 antibody showed a very robust response to a range of β-galactose. The dose response curve for galactosylation is shown in FIG. 5. In addition to yielding quantitatively very different activity levels, there was also a qualitative change in the shape of the dose response curve to the β-galactose levels, with the Hill slope and Emax dropping with decreasing β-galactose levels. See materials and method above for a description of calculating relative activity.

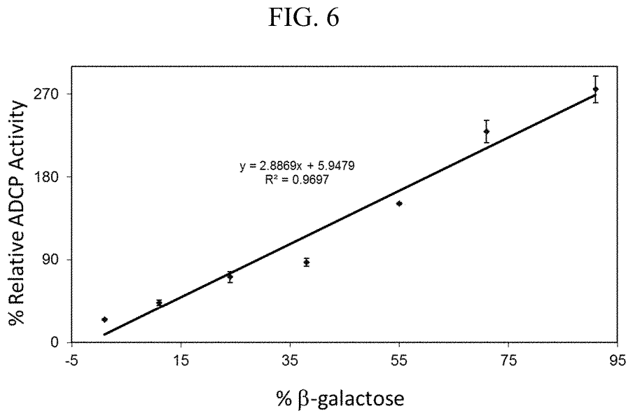

The calculated activity yielded a very linear response to the glycans FIG. 6. We were also able to quantify the relative impact of β-galactose on ADCP activity by expressing it in terms of the slope of the activity/attribute correlation plot, which can be taken to represent a response coefficient. Using this approach for the anti-HER2 antibody, the ADCP impact can be calculated as 2.88 for β-galactose.

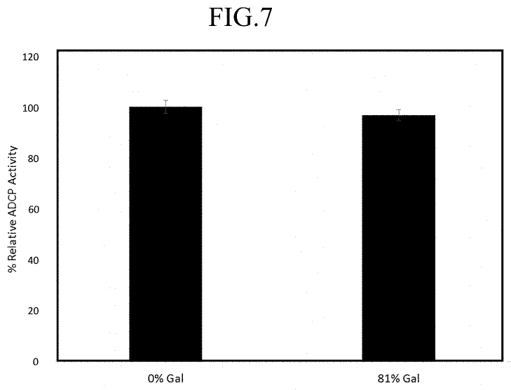

Surprisingly, when the same analysis was carried out for the anti-CD20 antibody, the response to β-galactose levels was found to be virtually flat as reported in FIG. 7. Since there was minimal impact observed between the lowest and highest achievable levels of (3-galactose for the anti-CD20 antibody, none of the intermediate levels were tested.

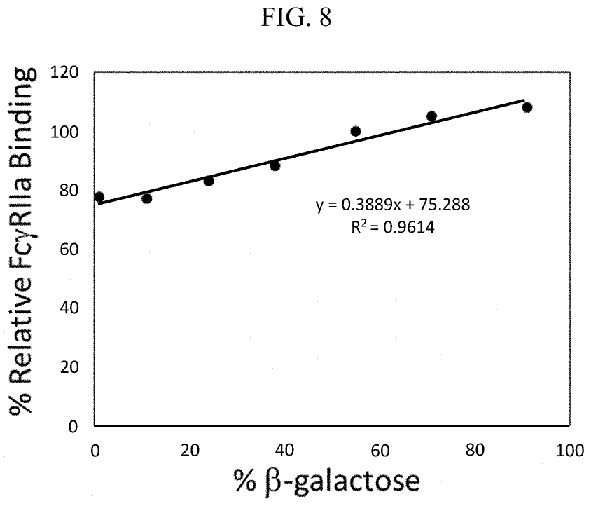

As a further measure of the responsiveness of the ADCP reporter gene assay to glycan levels, a FcγRIIa binding assay using an AlphaLISA format was conducted to provide an orthogonal assessment the impact of β-galactose for the anti-HER2 antibody. The FcγRIIa binding assay was qualified to cover a wide range of binding activity similar to the linearity approach used for the ADCP assay (data not shown). As can be seen in FIG. 8, with a subset of the samples covering the same range of β-galactose, there was a linear response to (3-galactose levels. It is interesting to note that the magnitude of the response for the AlphaLISA binding assay was far more modest than the response that the ADCP assay generates. The slope of the plot of relative FcγRIIa binding as a function of β-galactose levels is only 0.33, as compared to the 3.05 response coefficient from FIG. 6, which represents approximately a 9-fold stronger response rate for the ADCP assay.

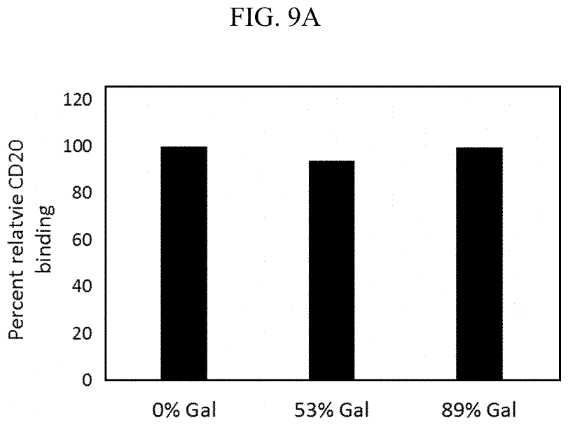

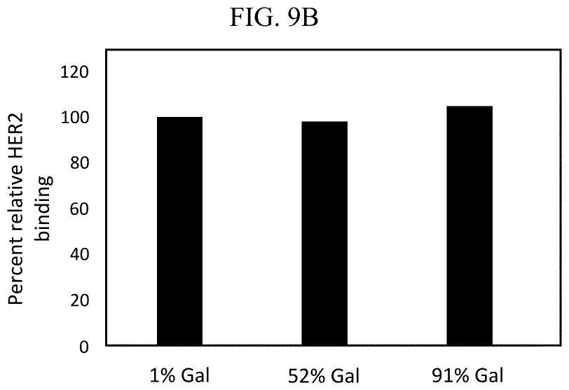

As a control for any antigen binding impact that may have occurred as a consequence of the glycoengineering manipulations, a graph of relative antigen binding activities for representative wide ranging β-galactose level samples are shown in FIGS. 9A-B. From these results it can be concluded that the differences in relative ADCP activity can be attributed to differences in FcγRIIa binding and are not due to inadvertent changes in cell surface target binding.

### Example 3

### Impact of Core Fucose/Afucosylation on ADCP Activity of IgG1 Antibodies

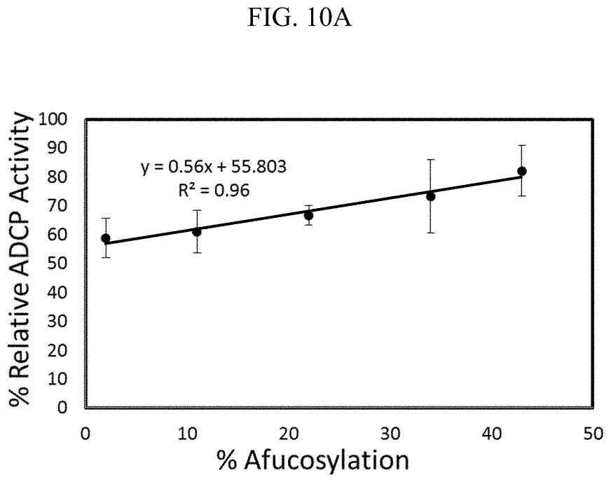

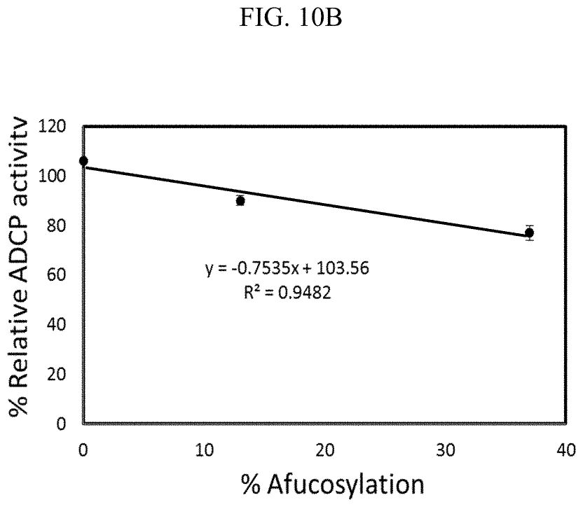

The responses to fucosylation (in the form of afucosylation [lack of fucose] levels) are shown in FIGS. 10A-B. We observed a linear increase in ADCP activity for the anti-HER2 antibody, though less dramatic than the response to β-galactose, with a response coefficient of 0.56. FIG. 10A. The anti-CD20 antibody showed a modest but linear decrease in ADCP activity with increasing afucosylation. FIG. 10B. The negative slope is very shallow but discernable and reproducible considering the precision of the method.

### Example 4

### Impact of High Mannose on ADCP Activity of IgG1 Antibodies

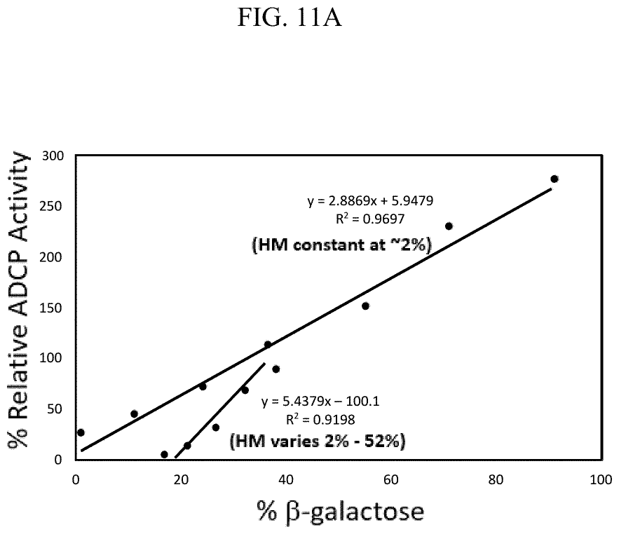

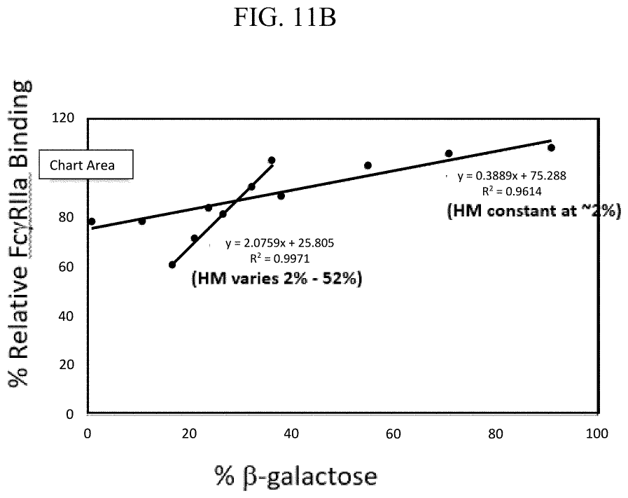

The response to high mannose for the anti-HER2 antibody was slightly confounded by the inability to fully separate lower level high mannose species from the higher level high mannose species by the affinity chromatography method without also carrying over the associated differing β-galactose levels for those samples. Ordinarily the (3-galactose would be removed for this assessment to enable the assessment of the impact due the high mannose differences alone. However, as was shown in the Example 2, removing (3-galactose abolishes activity. Therefore, to assess the impact of high mannose on the ADCP activity for the anti-HER2 antibody, two sets of samples were compared. The first set is the same β-galactose levels that range from 1% to 91% discussed in Example 20 but that all contain a common level of high mannose (˜2%). The second set contains samples with range from 17% β-galactose to 36% β-galactose (with intermediate levels achieved by blends of the two) but also contain a range of high mannose from 1.6% to 52%. The reported ADCP activity from each set relative to β-galactose are plotted in FIGS. 11A-B. We observed that the set containing a range of high mannose from 1.2-52% all showed lower ADCP activity relative to the set that had high mannose fixed at 2%, indicating that there is a negative impact of high mannose on the ADCP activity for the anti-HER2 antibody. FIG. 11A. The same analysis was done using the FcγRIIa binding assay and is shown in FIG. 11B. Since the slope of the sample set containing a range of high mannose is also influence by the range of β-galactose present on those samples, the value of the slope cannot be taken as the response coefficient for high mannose as it is for other glycan attributes. Rather, knowing the relationship between β-galactose and high mannose on those samples, as well as the relationship between β-galactose and ADCP activity, it was possible to calculate a response coefficient for high mannose of −2.11 for the ADCP activity and −0.81 for the binding assay.

The calculations to determine the response coefficient for high mannose in the ADCP assay were as follows:
% Relative ADCP activity as Function of % β-galactose

y=5.4379x−100.1

x=(y+100.1)/5.4379 (1)
Re-write the % β-galactose vs. % Mannose equation in terms of z, z being % Mannose level.

y=−0.3881x+36.9610.3881x

x=−0.3881z+36.961 (2)
Now substitute (1) in (2) to yield the equation for y (% Relative FcγRIIa Binding) in terms of z (% Mannose level)

$$\begin{matrix}x = - 0.3881 z + 36.9 \; 61 \\ \frac{( y + 100.1 )}{5.4379} = - 0.3881 z + 36.961 \\ [ \frac{( y + 100.1 )}{5.437 \; 9} = - 0.3881 z + 36.961 ] * 5.4379 \\ y + 100.1 = - 2.1104 z + 200 .9902 & \\ y = - 2.1104 z + 100.8902 & ( 3 )\end{matrix}$$

Therefore, the response coefficient for high mannose in the ADCP assay is −2.1104

The calculations to determine the response coefficient for high mannose in the FcγRIIa binding assay were as follows:
% Relative FcγRIIa Binding as Function of % β-galactose

y=2.0759x2.0759x+25.805

x=(y−25.805)/2.0759 (1)
Re-write the % β-galactose vs. % Mannose equation in terms of z, z being % Mannose level.

y=−0.3881x+36.961

x=−0.3881z+36.961 (2)
Now substitute (1) in (2) to yield the equation for y (% Relative FcγRIIa Binding) in terms of z (% Mannose level)

$$\begin{matrix}x = - 0.3881 z + 36.9 \; 61 \\ \frac{( y - 25.805 )}{2.0759} = - 0.3881 z + 36.961 \\ [ \frac{( y - 25.805 )}{2.075 \; 9} = - 0.3881 z + 36.961 ] * 2.0759 \\ y - 25.805 = - 0.805657 z + 76.7273 & \\ y = - 0.805657 z + 102.5323 & ( 3 )\end{matrix}$$

Therefore, the response coefficient for high mannose in the FcγRIIa binding is −0.81

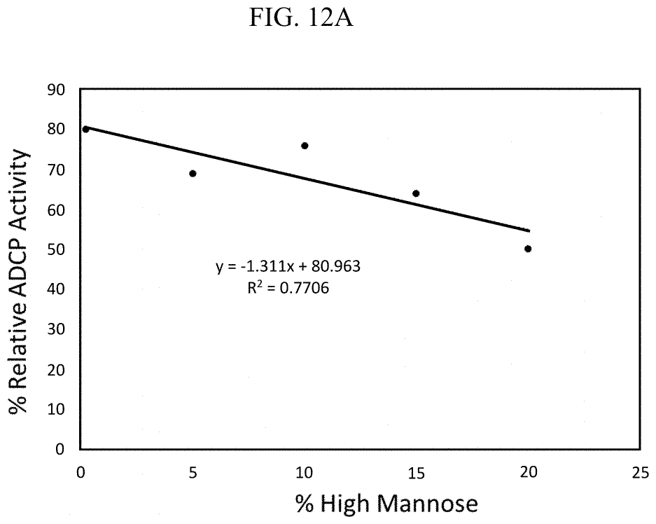

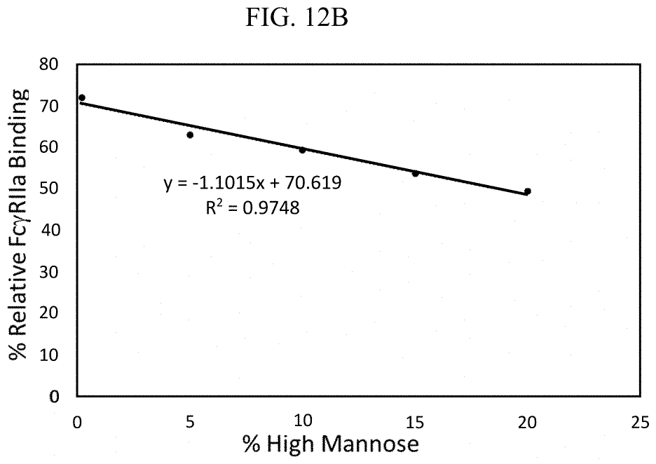

Samples for the assessment of high mannose on the anti-CD20 antibody were simpler to engineer since β-galactose does not have a significant impact on the ADCP activity. Similar to what was found for fucosylation, there is a slight linear decrease in ADCP activity with increasing levels of high mannose for the anti-CD20 antibody, as shown in FIG. 12A. Also shown in FIG. 12B is the corresponding FcγRIIa AlphaLISA binding assay results showing a similar negative correlation with high mannose. Here again, the ADCP assay displays a greater magnitude of glycan effect, albeit slightly, than the orthogonal binding assay.

### Discussion

A summary of the impact for the various glycan species towards ADCP activity as measured by response coefficients for the CD20 and HER2 antibodies is summarized in Table 3. Increased ADCP activity (as shown by positive numbers in Table 3) or decreased ADCP activity (as shown by negative numbers in Table 3) were observed when the amount of specific glycans (a-fucose, high mannose or terminal β-galactose) were increased in each antibody.

TABLE 3
Summary of ADCP Activity

| Glycan | Anti-CD20 | Anti-HER2 |
|---|---|---|
| a-fucose (lack of core fucose) | −0.75 | 0.56 |
| High mannose | −1.31 | −2.11 |
| Terminal β-galactose | None | 2.88 |

In the manufacture of therapeutic antibodies, it is crucial to identify and characterize all of the effector function activities that the molecule is capable of mediating. While ADCC and CDC assays are well established, ADCP is easy to overlook as an activity and possible mechanism of action due to the more limited sensitivity and range of the assays required and less will understood quality attributes that affect activity. However, since ADCP has been suggested as a possible mechanism of action for at least a couple of currently marketed therapeutic monoclonal antibodies (Kim S, et al. Drifts in ADCC-related quality attributes of Herceptin(R): Impact on development of a trastuzumab biosimilar. mAbs 2017; 9:704-14; and Weng W K, Levy R. Two immunoglobulin G fragment C receptor polymorphisms independently predict response to rituximab in patients with follicular lymphoma. Journal of clinical oncology: official journal of the American Society of Clinical Oncology 2003; 21:3940-7) it is crucial to monitor critical quality attributes that can impact ADCP activity. Indeed, for molecules the display any of these activities, it will be essential to elucidate these structure/function relationships to ensure appropriate process control as well as for the demonstration of analytical similarity in the development of biosimilars. Central to achieving this aim is the development of functional assays sensitive to changes in quality attributes that can vary as part of the manufacturing process. In the present study we made use of a combined enzymatic modification and enrichment process to generate material from two representative therapeutic monoclonal antibodies produced in a CHO production process to yield wide ranges of three main glycan species, each in isolation. We were able to achieve ranges substantially wider than would be possible by adjusting process parameters in order to more accurately discern the relationship between glycan species and effector activity.

The nature of the interactions between IgG antibody Fc domains and Fcγ-receptor family members has been extensively reviewed in Reusch D, Tejada M L. Fc glycans of therapeutic antibodies as critical quality attributes. Glycobiology 2015; 25:1325-34. While the role of the conserved glycan and the various structural forms has been well established through binding and functional studies for FcγRIIIa and ADCC, the impact of glycan structures on FcγRIIa and ADCP has been less clearly defined. While most studies have not found a substantial relationship between the structure of the conserved N-linked glycan and FcγRIIa binding and ADCP, there have been a few reports observing a correlation. Chung et al (see Chung S, et al. Quantitative evaluation of fucose reducing effects in a humanized antibody on Fcgamma receptor binding and antibody-dependent cell-mediated cytotoxicity activities. mAbs 2012; 4:326-40) found that in the case of an anti-CD20 antibody, there was a trend of increasing FcγRIIa binding with increasing afucosylation levels from 0-100%. Those data were derived from testing blended samples from a DP12 (wild type generating) and a FUT8 deficient (afucose yielding) CHO cell line in an ELISA binding assay. While we observe a small increase for the anti-HER2 antibody with increasing levels of afucosylation in the reporter gene ADCP assay, we in fact find a small decrease in activity for the anti-CD20 antibody. It's possible that the differences in assay format, as well as the fact that Chung et al use the 131R allotype in their binding study whereas this study used the 131H allotype, may have contributed to the divergent observations. The outcomes may also have been impacted by the possible use of a different anti-CD20 molecule used for the studies as well as the possibility that there may be other structural differences deriving from the FUT8 deficient host that aren't represented in the in vitro engineering process employed in this study. Of note, another study (Golay J, et al. Glycoengineered CD20 antibody obinutuzumab activates neutrophils and mediates phagocytosis through CD16B more efficiently than rituximab. Blood 2013; 122:3482-91) used an anti-CD20 antibody that was glycoengineered through production in a CHO line with inducible N-Acetylglucosaminyltransferase (GntIII) expression, which yields bisecting glycans that are not substrates for fucosyltransferase and therefore are largely expressed as afucosylated forms. See Umana P, et al. Engineered glycoforms of an antineuroblastoma IgG1 with optimized antibody-dependent cellular cytotoxic activity. Nature biotechnology 1999; 17:176-80. In that study the authors observed essentially no impact from the fucose level for FcγRIIa binding for the 131R allotype, but a small decrease in binding to the 131H allotype as measured by surface plasmon resonance, which would be consistent with our study. Galactose levels have been identified as impactful in a recent study (see Chung A W et al. Identification of antibody glycosylation structures that predict monoclonal antibody Fc-effector function. Aids 2014; 28:2523-30) in which the authors were able to observe an increase in a human macrophage cell line ADCP assay, as well as in an SPR binding assay for FcγRIIa binding, for antibodies with higher levels of terminal sugars, including galactose. Unique to those experiments, the glycan ranges were all on different mouse monoclonal antibodies where alpha linkages prevail, rather than beta linkages used in this study and which are present on the much more commonly used CHO host cell lines. Another study looked at the effect of galactose levels on a murine monoclonal for binding to FcγRIIb, a structurally related receptor to FcγRIIa (see Woof J M, Burton D R. Human antibody-Fc receptor interactions illuminated by crystal structures. Nature reviews Immunology 2004; 4:89-99), but with an inhibitory intracellular signaling motif. In several in vivo assays for neutrophil activity thought to be mediated through FcγRIIa those authors observed much higher activity for a fully enzymatically galactosylated than a fully enzymatically degalactosylated form of the molecule. Karsten C M et al. Anti-inflammatory activity of IgG1 mediated by Fc galactosylation and association of FcgammaRIIB and dectin-1. Nature medicine 2012; 18:1401-6. So while there have been some efforts to look for the impact to glycan structures on FcγRIIa binding and signaling activity, this report appears to be the first effort to systematically evaluate multiple differently glycan species in a cell based assay for FcγRIIa binding.

Perhaps the biggest challenge in studying structural determinants for ADCP activity has been the reliance on primary effector cells, which are susceptible to donor to donor response variability, as well as receptor allotype differences and other background genetic and phenotypic variables that could affect assay output. Also, it's generally more difficult to discern the relevant receptors for ADCP on phagocytes since multiple different FcγR types can be expressed on the effector cells of interest, as others have also noted. See, Ackerman M E, et al. A robust, high-throughput assay to determine the phagocytic activity of clinical antibody samples. Journal of immunological methods 2011; 366:8-19. Consequently, the limitations of these types of assays are in drug development and characterization in a quality-control setting should be recognized. In an effort to avoid these challenges, Tada et el (Tada M, et al. Development of a cell-based assay measuring the activation of FcgammaRIIa for the characterization of therapeutic monoclonal antibodies. PloS one 2014; 9:e95787) developed a reporter gene assay for FcγRIIa mediated ADCP, which overcomes many of the above limitations. In addition to demonstrating the utility and convenience of using a reporter gene assay for ADCP activity, they found an impact to ADCP activity by methionine oxidation of cetuximab.

Using a similar assay methodology, we report for the first time that common glycoforms can have substantial and differential impact to ADCP activity between IgG1 mAbs. Most impressive is the β-galactose impact on the anti-HER2 antibody. Even when the terminal β-galactose level range is limited to levels observed during a typical manufacturing process, a 10-fold change in activity was observed. A similar magnitude of impact but of opposite direction (inhibitory with increasing levels) was interestingly found for high mannose on this antibody. Such results could inform a control strategy for any such molecule where ADCP was part of the proposed mechanism of action. Interestingly, for the anti-CD20 antibody, virtually no impact was observed from a similarly large range of terminal β-galactose levels. In contrast, for that same molecule, the impact of afucosylation and high mannose is opposite of what is generally predicted for FcγRIIIa binding and ADCC. The structural basis for those glycans' impact on ADCC was made clear from the co-crystal structure of FcgRIIIa with an IgG1 Fc domain. See, Ferrara C, et al. Unique carbohydrate-carbohydrate interactions are required for high affinity binding between FcgammaRIII and antibodies lacking core fucose. Proceedings of the National Academy of Sciences of the United States of America 2011; 108:12669-74. If similar structural studies for FcγRIIa binding were to be performed, they may prove to be equally illuminating.

As a final observation, the results presented here show that where a glycan impact is observed, the magnitude of the response can be greater than the magnitude reported by an orthogonal AlphaLISA binding assay, which itself is generally more responsive than SPR or ELISA assay formats to binding differences. See Xu Z, et al. Affinity and cross-reactivity engineering of CTLA4-Ig to modulate T cell costimulation. Journal of immunology 2012; 189:4470-7. The responsiveness seen in this ADCP assay may be unique when one considers that reports of ADCC-impacting attributes which make use of both cell-based and binding assays find that the binding assays are more responsive than the cell-based activity assays. See, Reusch D, Tejada M L. Fc glycans of therapeutic antibodies as critical quality attributes. Glycobiology 2015; 25:1325-34; see also, Chung S, et al. Characterization of in vitro antibody-dependent cell-mediated cytotoxicity activity of therapeutic antibodies—impact of effector cells. Journal of immunological methods 2014; 407:63-75. This fact highlights further the potential sensitivity and utility of this assay for FcγRIIa binding.

In conclusion, not all IgG1 mAbs directed at cell surface targets are going to have the same potential to elicit all effector functions, and where activity is present, quality attributes can have a differential impact on the different effector functions. This emphasizes the importance of assessing for ADCP activity just diligently as ADCC and CDC activity assessments, and in controlling the relevant critical quality attributes in manufacturing processes. Also highlighted is the need for establishing appropriate controls on a case-by-case basis, as platform control strategies may not be adequate. Toward that end we highlight the value of having a high-resolution assay with high throughput potential that can reveal CQAs relevant to effector functions that might otherwise have been overlooked. While the desire to pursue physiologically relevant assays may lead to a reliance on primary cells, the best approach may be to apply a combination of primary cell assays to provide physiological context for hypothesis testing and robust, sensitive, high-throughput reporter gene assays to establish production parameters.

### Selective References

1. Jiang X R, Song A, Bergelson S, Arroll T, Parekh B, May K, et al. Advances in the assessment and control of the effector functions of therapeutic antibodies. Nature reviews Drug discovery 2011; 10:101-11.
2. Reusch D, Tejada M L. Fc glycans of therapeutic antibodies as critical quality attributes. Glycobiology 2015; 25:1325-34.
3. Okazaki A, Shoji-Hosaka E, Nakamura K, Wakitani M, Uchida K, Kakita S, et al. Fucose depletion from human IgG1 oligosaccharide enhances binding enthalpy and association rate between IgG1 and FcgammaRIIIa. Journal of molecular biology 2004; 336:1239-49.
4. Ferrara C, Grau S, Jager C, Sondermann P, Brunker P, Waldhauer I, et al. Unique carbohydrate-carbohydrate interactions are required for high affinity binding between FcgammaRIII and antibodies lacking core fucose. Proceedings of the National Academy of Sciences of the United States of America 2011; 108:12669-74.
5. Thomann M, Reckermann K, Reusch D, Prasser J, Tejada M L. Fc-galactosylation modulates antibody-dependent cellular cytotoxicity of therapeutic antibodies. Molecular immunology 2016; 73:69-75.
6. Hodoniczky J, Zheng Y Z, James D C. Control of recombinant monoclonal antibody effector functions by Fc N-glycan remodeling in vitro. Biotechnology progress 2005; 21:1644-52.
7. Shinkawa T, Nakamura K, Yamane N, Shoji-Hosaka E, Kanda Y, Sakurada M, et al. The absence of fucose but not the presence of galactose or bisecting N-acetylglucosamine of human IgG1 complex-type oligosaccharides shows the critical role of enhancing antibody-dependent cellular cytotoxicity. The Journal of biological chemistry 2003; 278:3466-73.
8. Umana P, Jean-Mairet J, Moudry R, Amstutz H, Bailey J E. Engineered glycoforms of an antineuroblastoma IgG1 with optimized antibody-dependent cellular cytotoxic activity. Nature biotechnology 1999; 17:176-80.
9. Kim S, Song J, Park S, Ham S, Paek K, Kang M, et al. Drifts in ADCC-related quality attributes of Herceptin(R): Impact on development of a trastuzumab biosimilar. mAbs 2017; 9:704-14.
10. Weng W K, Levy R. Two immunoglobulin G fragment C receptor polymorphisms independently predict response to rituximab in patients with follicular lymphoma. Journal of clinical oncology: official journal of the American Society of Clinical Oncology 2003; 21:3940-7.
11. Zhang W, Gordon M, Schultheis A M, Yang D Y, Nagashima F, Azuma M, et al. FCGR2A and FCGR3A polymorphisms associated with clinical outcome of epidermal growth factor receptor expressing metastatic colorectal cancer patients treated with single-agent cetuximab. Journal of clinical oncology: official journal of the American Society of Clinical Oncology 2007; 25:3712-8.
12. Petricevic B, Laengle J, Singer J, Sachet M, Fazekas J, Steger G, et al. Trastuzumab mediates antibody-dependent cell-mediated cytotoxicity and phagocytosis to the same extent in both adjuvant and metastatic HER2/neu breast cancer patients. Journal of translational medicine 2013; 11:307.
13. Tada M, Ishii-Watabe A, Suzuki T, Kawasaki N. Development of a cell-based assay measuring the activation of FcgammaRIIa for the characterization of therapeutic monoclonal antibodies. PloS one 2014; 9:e95787.
14. Read E K, Park J T, Brorson K A. Industry and regulatory experience of the glycosylation of monoclonal antibodies. Biotechnology and applied biochemistry 2011; 58:213-9.
15. Arora T, Padaki R, Liu L, Hamburger A E, Ellison A R, Stevens S R, et al. Differences in binding and effector functions between classes of TNF antagonists. Cytokine 2009; 45:124-31.
16. Chung S, Quarmby V, Gao X, Ying Y, Lin L, Reed C, et al. Quantitative evaluation of fucose reducing effects in a humanized antibody on Fcgamma receptor binding and antibody-dependent cell-mediated cytotoxicity activities. mAbs 2012; 4:326-40.
17. Golay J, Da Roit F, Bologna L, Ferrara C, Leusen J H, Rambaldi A, et al. Glycoengineered CD20 antibody obinutuzumab activates neutrophils and mediates phagocytosis through CD16B more efficiently than rituximab. Blood 2013; 122:3482-91.
18. Chung A W, Crispin M, Pritchard L, Robinson H, Gorny M K, Yu X, et al. Identification of antibody glycosylation structures that predict monoclonal antibody Fc-effector function. Aids 2014; 28:2523-30.
19. Woof J M, Burton D R. Human antibody-Fc receptor interactions illuminated by crystal structures. Nature reviews Immunology 2004; 4:89-99.
20. Karsten C M, Pandey M K, Figge J, Kilchenstein R, Taylor P R, Rosas M, et al. Anti-inflammatory activity of IgG1 mediated by Fc galactosylation and association of FcgammaRIIB and dectin-1. Nature medicine 2012; 18:1401-6.
21. Ackerman M E, Moldt B, Wyatt R T, Dugast A S, McAndrew E, Tsoukas S, et al. A robust, high-throughput assay to determine the phagocytic activity of clinical antibody samples. Journal of immunological methods 2011; 366:8-19.
22. Xu Z, Juan V, Ivanov A, Ma Z, Polakoff D, Powers D B, et al. Affinity and cross-reactivity engineering of CTLA4-Ig to modulate T cell costimulation. Journal of immunology 2012; 189:4470-7.
23. Chung S, Lin Y L, Reed C, Ng C, Cheng Z J, Malavasi F, et al. Characterization of in vitro antibody-dependent cell-mediated cytotoxicity activity of therapeutic antibodies—impact of effector cells. Journal of immunological methods 2014; 407:63-75.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this disclosure that certain changes and modifications may be made thereto without departing from the spirit or scope of the disclosed embodiments. The section headings used herein are for organizational purposes only and are not to be construed as limiting the subject matter described.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. (canceled)

2. (canceled)

3. (canceled)

4. (canceled)

5. (canceled)

6. (canceled)

7. (canceled)

8. (canceled)

9. (canceled)

10. A method of modulating Antibody Dependent Cellular Phagocytosis (ADCP) activity of trastuzumab or rituximab comprising increasing or decreasing the amount of core fucose in the antibody; or increasing or decreasing the amount of fucosylated species of the antibody.

11. A method of increasing Antibody Dependent Cellular Phagocytosis (ADCP) activity of trastuzumab comprising decreasing the amount of core fucose in the antibody; or increasing the amount of afucosylated species of the antibody.

12. The method of claim 11, wherein a decrease of about 1 percent of core fucose increases ADCP activity of the trastuzumab antibody by about 0.5, about 0.56, about 0.6 or about 1 percent.

13. A method of increasing Antibody Dependent Cellular Phagocytosis (ADCP) activity of rituximab comprising increasing the amount of core fucose in the antibody or decreasing the amount of afucosylated species of the antibody.

14. The method of claim 13, wherein an increase of about 1 percent of core fucose increases ADCP activity of rituximab by about 0.5, about 0.7, about 0.75, about 0.8 or about 1 percent.

15. A method of decreasing Antibody Dependent Cellular Phagocytosis (ADCP) activity of trastuzumab comprising increasing the amount of core fucose in the antibody or decreasing the amount of afucosylated species of the antibody.

16. The method of claim 15, wherein an increase of about 1 percent of core fucose decreases ADCP activity of the trastuzumab antibody by about 0.5, about 0.56, about 0.6 or about 1 percent.

17. A method of decreasing Antibody Dependent Cellular Phagocytosis (ADCP) activity of rituximab comprising decreasing the amount of core fucose in the antibody or increasing the amount of afucosylated species of the antibody.

18. The method of claim 17, wherein a decrease of about 1 percent of core fucose decreases ADCP activity of rituximab by about 0.5, about 0.7, about 0.75, about 0.8 or about 1 percent.

19. A method of matching Antibody Dependent Cellular Phagocytosis (ADCP) activity of an IgG1 antibody comprising: (1) determining the ADCP activity of a reference IgG1 antibody; (2) determining the ADCP activity of a second IgG1 antibody having the same sequence as the reference IgG1 antibody; and (3) changing the ADCP activity of the second IgG1 antibody by increasing or decreasing the amount of core fucose in the second antibody or increasing or decreasing the amount of afucosylated species of the second antibody, wherein the ADCP activity of the second antibody after increasing or decreasing the amount of core fucose or increasing or decreasing the amount of afucosylated species is the same as the reference antibody or within about 10%, about 15%, about 20%, about 25%, about 30% or about 35% of the reference antibody or within the range of about 1% to about 35% of the reference antibody.

20. The method of claim 19, wherein the reference and second IgG1 antibodies are trastuzumab and the ADCP activity of the second antibody is increased by decreasing the amount of core fucose in the second antibody or increasing the amount of afucosylated species of the second antibody, wherein the ADCP activity of the second antibody after increasing ADCP activity is the same as the reference antibody or within about 10%, about 15%, about 20%, about 25%, about 30% or about 35% of the reference antibody or within the range of about 1% to about 35% of the reference antibody.

21. The method of claim 19, wherein the reference and second IgG1 antibodies are trastuzumab and the ADCP activity of the second trastuzumab antibody is decreased by increasing the amount of core fucose in the second antibody or decreasing the amount of afucosylated species of the second antibody, wherein the ADCP activity of the second antibody after decreasing ADCP activity is the same as the reference antibody or within about 10%, about 15%, about 20%, about 25%, about 30% or about 35% of the reference antibody or within the range of about 1% to about 35% of the reference antibody.

22. The method of claim 19, wherein the reference and second IgG1 antibodies are rituximab and the ADCP activity of the second rituximab antibody is increased by increasing the amount of core fucose in the second antibody or decreasing the amount of afucosylated species of the second antibody, wherein the ADCP activity of the second antibody after increasing ADCP activity is the same as the reference antibody or within about 10%, about 15%, about 20%, about 25%, about 30% or about 35% of the reference antibody or within the range of about 1% to about 35% of the reference antibody.

23. The method of claim 19, wherein the reference and second IgG1 antibodies are rituximab and the ADCP activity of the second rituximab antibody is decreased by decreasing the amount of core fucose in the second antibody or increasing the amount of afucosylated species of the second antibody, wherein the ADCP activity of the second antibody after decreasing ADCP activity is the same as the reference antibody or within about 10%, about 15%, about 20%, about 25%, about 30% or about 35% of the reference antibody or within the range of about 1% to about 35% of the reference antibody.

24. A method of engineering a specific target Antibody Dependent Cellular Phagocytosis (ADCP) activity of a trastuzumab antibody comprising: (1) determining the ADCP activity of a trastuzumab antibody; (2) determining a target ADCP activity; and (3) changing the ADCP activity of the trastuzumab antibody by increasing or decreasing the amount of core fucose in the second antibody or increasing or decreasing the amount of afucosylated species of the second antibody, wherein the ADCP activity of the antibody after increasing or decreasing the amount of core fucose or increasing or decreasing the amount of afucosylated species is the same as the target ADCP activity or within about 10%, about 15%, about 20%, about 25%, about 30% or about 35% of the target ADCP activity or within the range of about 1% to about 35% of the target ADCP activity.

25. A method of engineering a specific target Antibody Dependent Cellular Phagocytosis (ADCP) activity of a rituximab antibody comprising: (1) determining the ADCP activity of a rituximab antibody; (2) determining a target ADCP activity; and (3) changing the ADCP activity of the rituximab antibody by increasing or decreasing the amount of core fucose in the second antibody or increasing or decreasing the amount of afucosylated species of the second antibody, wherein the ADCP activity of the antibody after increasing or decreasing the amount of core fucose or increasing or decreasing the amount of afucosylated species is the same as the target ADCP activity or within about 10%, about 15%, about 20%, about 25%, about 30% or about 35% of the target ADCP activity or within the range of about 1% to about 35% of the target ADCP activity.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)
